(19) United States
(12) Patent Application Publication
(10) Pub. No.: US 2024/0251800 A1
(43) Pub. Date: Aug. 1, 2024

(54) ANTIBACTERIAL METHODS & CELLS

(71) Applicant: Folium Food Science Limited, Bristol (GB)

(72) Inventors: Simon WARNER, Bristol (GB); Abiyad BAIG, Bristol (GB); Jasper CLUBE, London (GB)

(21) Appl. No.: 18/281,265

(22) PCT Filed: Nov. 8, 2021
(86) PCT No.: PCT/EP2021/080876

§ 371 Date: Sep. 8, 2023

(30) Foreign Application Priority Data
Nov. 8, 2020 (GB) 2017618.6

(51) Int. Cl. A01N 63/20

(52) U.S. Cl. CPC A01N 63/20

(57) ABSTRACT

The invention relates to methods of killing bacterial target cells comprising Resistance-Nodulation-Cell Division (RND)-efflux pumps, as well as carrier cells useful for this purpose wherein the carrier cells comprise a conjugative plasmid encoding an antibacterial-microbial agent that is toxic to target cells. A carrier bacterium is capable of conjugative transfer of plasmid DNA encoding the agent to a target cell.

### TECHNICAL FIELD

The invention relates to methods of killing bacterial target cells comprising Resistance-Nodulation-Cell Division (RND)-efflux pumps, as well as carrier cells useful for this purpose wherein the carrier cells comprise a conjugative plasmid encoding an antibacterial-microbial agent that is toxic to target cells. A carrier bacterium is capable of conjugative transfer of plasmid DNA encoding the agent to a target cell.

### BACKGROUND

### Bacterial Efflux Systems as Determinants of Multidrug Resistance

Efflux pumps are bacterial transport proteins which are involved in extrusion of substrates from the cellular interior to the external environment. These substrates are often antibiotics, imparting the efflux pump expressing bacteria antibiotic resistant phenotype. From the first drug-resistant efflux pump discovered in the 1990s, the development in molecular microbiology has led to the characterization of many efflux pumps in Gram-positive bacteria (GPB) including methicillin-resistant Staphylococcus aureus (MRSA), Streptococcus pneumoniae, Clostridium difficile, Enterococcus spp. and Listeria monocytogenes and Gram-negative bacteria (GNB) such as Acinetobacter baumannii, Escherichia coli, Klebsiella pneumoniae, Stenotrophomonas maltophilia, Campylobacter jejuni, Pseudomonas aeruginosa, Neisseria gonorrhoeae, Vibrio cholerae and Salmonella spp. Since these transport substrates against a concentration gradient, these efflux pumps are energy dependent. Based on the mechanism by which these derive this energy, the efflux pumps are broadly classified into two categories. The primary efflux pumps draw energy from active hydrolysis of ATP, whereas the secondary efflux pumps draw energy from chemical gradients formed by either protons or ions such as sodium. Five major families of efflux pumps have been described in the prokaryotes, namely: (i) ATP binding cassette (ABC), which are primary active transporters, (ii) small multidrug resistance family, (iii) multidrug and toxin extrusion (MATE) family, (iv) major facilitator superfamily (MFS) and (v) resistance nodulation cell division (RND) family. RND family efflux pumps have tripartite organization and are the major contributors to intrinsic antibiotic resistance, which expel a broad spectrum of antibiotics and biocides, including fluoroquinolones, β-lactams, tetracycline and linezolid. Apart from drug resistance, the physiological role of efflux pumps in bacteria extends to bile tolerance in enteric bacteria, leading to colonization, increase in virulence, biofilm secretion and bacterial survival in the host.

Biofilms are complex microbial associations anchored to abiotic or biotic surfaces, embedded in extracellular matrix produced by the biofilms themselves where they interact with each other and the environment. One of the main properties of biofilms is their capacity to be more resistant to antimicrobial agents than planktonic cells. Efflux pumps have been reported as one of the mechanisms responsible for the antimicrobial resistance in biofilm structures. Evidence of the role of efflux pump in biofilm resistance has been found in several microorganisms such as Pseudomonas aeruginosa, Escherichia coli and Candida albicans.

### RND Efflux Pumps

Multidrug efflux pumps belonging to the resistance-nodulation cell division (RND) family have major roles in the intrinsic and elevated resistance of Gram-negative bacteria to a wide range of compounds. RND efflux pumps require two other proteins to function: a membrane fusion protein (MFP) and an outer membrane protein. It has been demonstrated that Salmonella enterica serovar Typhimurium has five RND efflux systems: AcrAB, AcrD, AcrEF, MdtABC and MdsABC. Most RND efflux system genes also code for an MFP in the same operon.

Efflux pumps belonging to the resistance-nodulation-division (RND) family of transporters are the major multi-drug efflux (Mex) mechanism in both E. coli and P. aeruginosa. The pumps in this family consist of three components that function via active transport to move numerous molecules, including antibiotics, out of the cell: an antiporter that functions as a transporter (e.g., MexB, Mex D, MexF, MexY), an outer membrane protein that forms a surface-exposed channel (e.g., OprC, OprB, OprG, OprD, Oprl, OprH, OprP, OprO, OprM, OprJ, OprN), and a periplasmic membrane fusion protein that links the two proteins (e.g., MexA, MexC, MexE, MexH, MexX). This system is the major efflux pump associated with intrinsic resistance among 17 possible RND efflux pumps in P. aeruginosa. P aeruginosa is more resistant than E. coli due to a highly impermeable OM and the presence of multiple efflux systems. Inactivation of the Mex efflux pump renders P. aeruginosa more vulnerable to antibiotics than the average E. coli strain.

The flavonoid-responsive RND family of efflux pumps includes several members, such as AcrAB from Erwinia amylovora, IfeAB from Agrobacterium tumefaciens, MexAB-OprM from Pseudomonas syringae, BjG30 from Bradyrhizobium japonicum, and EmrAB in Sinorhizobium meliloti, among others. Further supporting the role of this efflux pump in bacteria/plant interactions, it has been reported that E. amylovora, an enterobacterium that causes fire blight on species of the Rosaceae family, has an AcrAB efflux pump, which confer resistance to phytoalexins, and that is required for successful colonization of the plants and for bacterial virulence. This finding is in agreement with the idea that the ability export toxic compounds is one of the key traits for survival in the rhizosphere, and efflux pumps may have a relevant role for achieving resistance to these toxic compounds.

Phylogenetically close to E. coli, the enterobacterial pathogen Salmonella enterica serovar Typhimurium presents at least nine multidrug efflux pumps. Among these pumps, AcrAB, the orthologue of the E. coli efflux pump with the same name, contributes to antimicrobial resistance and has a wide substrate spectrum that includes antibiotics, dyes, and detergents. Another important gut pathogen is Campylobacter jejuni. Among the known antibiotic resistance mechanisms of this microorganism, the CmeABC efflux pump is a relevant player and confers resistance to structurally-diverse antibiotics and toxic compounds, including those naturally present in its animal host, as bile salts. CmeABC belongs to the RND family of efflux transporters and its expression is regulated by the transcriptional repressor CmeR, which binds to a specific site in the promoter region of cmeABC. Free-living bacteria, including opportunistic pathogens with an environmental origin, should respond to different signals and this may impact their behaviour in clinical and non-clinical ecosystems. For instance, Pseudomonas aeruginosa express several RND-type efflux systems, among which four, MexAB-OprM, MexCD-OprJ, MexEF-OprN, and MexXY-OprM are reported to be significant determinants of multidrug resistance.

The fact that the expression of MDR efflux pumps is induced by host-produced compounds suggests that they can play a role in the virulence of bacterial pathogens. Indeed, it has been shown that the Vibrio cholerae efflux pump VexB is the primary efflux system responsible for resistance to bile salts in this microorganism. Since bile salts are present at the human gut, the activity of this efflux pump is a pre-requisite for V. cholerae infection. A similar situation happens with AcrAB, the main pump responsible for bile salts resistance in Enterobacteriaceae, which is required for the pathogenesis of Salmonella enterica serovar Typhimurium. Notably this efflux pump is involved as well in the bacterial capability for forming biofilms. A protective role to host antibacterial compounds has also been described in the case of Neisseria gonorrhoeae. In this organism, the MtrCDE efflux pump contributes to resistance to vertebrate antibacterial peptides, and FarAB is involved in resistance to long-chain fatty acids. The activity of these efflux pumps contributes to the pathogenesis of N. gonorrhoeae. Similarly, the Campylobacter jejuni CmeABC efflux pump confers resistance to bile salts, fatty acids, and detergents, and is needed for the colonization of the intestinal tract.

Together with their role in modulating the quorum-sensing response, and consequently bacterial virulence, these results support the notion that MDR efflux pumps, besides contributing to the resistance of bacterial pathogens, are major contributors to their pathogenicity.

Pseudomonas syringae pv. tomato DC3000 (PsPto) is a phytopatogenic bacterium that infects tomato (causing bacterial speck) and Arabidopsis thaliana. PsPto can grow epiphytically and endophytically on plant foliage without causing disease symptoms. In the early stages of the infective phase, PsPto enters the plant through wounds and natural openings (such as stomata) and multiplies in the apoplastic space by exploiting live host cells. In this scenario, bacterial survival in the apoplast is one of the key factors for the establishment of a bacterial density large enough to further infect adjacent plant tissues. However, plant apoplast represents a harsh environment for bacteria since it is laden with antimicrobial compounds, both preformed (phytoanticipins) and inducible (phytoalexins), which constitute chemical barriers capable of inhibiting the growth of the pathogen. In fact, plants produce antimicrobial peptides and a variety of secondary metabolites such as phenylpropanoids, isoprenoids, and alkaloids, that are generally accepted to play a role in protecting plants against pathogens. Using the tomato-PsPto pathosystem, an increased expression of phenylpropanoid biosynthetic genes has been detected upon bacterial infection, with specific accumulation of different phenylpropanoids such as hydroxycinnamic acid amides conjugated to alkaloids, chlorogenic acid (CGA), and the flavonoid rutin. Tomato plants have also been reported to produce other number of flavonoids like chalconaringenin, rutin, quercetin 3-O-(2″-O-β-apiosyl-6″-O-α-rhamnosyl-β-glucoside) or phloretin 3′, 5′-di-C-β-glucoside. To overcome the effect of these potentially toxic compounds, plant-associated bacteria have in turn evolved different defense strategies, among which multidrug resistance (MDR) efflux pumps are the most widespread. MDR transporters can recognize and pump out many different organic compounds (often structurally dissimilar), providing resistance to antibiotics and many other antimicrobial compounds. Microorganisms with the largest number of MDR pumps are found in the soil or in association with plants. Although still scarce, several studies on plant-pathogen interactions with bacteria from the genera Xanthomonas, Ralstonia, Erwinia and Dickeya have shown that efflux pumps can contribute to bacterial virulence, bacterial fitness, resistance to plant antimicrobials, or competition with epiphytic bacteria.

Regarding P. syringae, most studies have been focused on MexAB-OprM, an efflux pump from the resistance-nodulation-cell division (RND) family. It has been shown that the P. syringae MexAB-OprM system is involved in the tolerance to a broad range of toxic compounds, including some plant-derived antimicrobials, and that a mutant in this system showed a reduced ability to multiply in planta. A recent study on the Arabidopsis-PsPto pathosystem has identified three RND efflux pumps (one of them the MexAB-OprM system) which are required to overcome the isothiocyanate-based defenses of Arabidopsis.

PSPTO_0820 is a predicted multidrug transporter from the phytopathogenic bacterium Pseudomonas syringae pv. tomato DC3000. Orthologs of this protein are conserved within many Pseudomonas species that interact with plants. Reference is made to PLoS One, 2019 Jun. 25:14(6): e0218815. doi: 10.1371/journal.pone.0218815. eCollection 2019, “The Pseudomonas syringae pv. tomato DC3000 PSPTO_0820 multidrug transporter is involved in resistance to plant antimicrobials and bacterial survival during tomato plant infection”, Saray Santamaria-Hernando et al: To study the potential role of PSPTO_0820 in plant-bacteria interaction, a mutant in this gene was isolated and characterized. In addition, with the aim to find the outer membrane channel for this efflux system, a mutant in PSPTO_4977, a TolC-like gene, was also analyzed. Both mutants were more susceptible to trans-cinnamic and chlorogenic acids and to the flavonoid (+)-catechin, when added to the culture medium. The expression level of both genes increased in the presence of (+)-catechin and, in the case of PSPTO_0820, also in response to trans-cinnamic acid. PSPTO_0820 and PSPTO 4977 mutants were unable to colonize tomato at high population levels. This work evidences the involvement of these two proteins in the resistance to plant antimicrobials, supporting also the importance of chlorogenic acid, trans-cinnamic acid, and (+)-catechin in the tomato plant defense response against P. syringae pv. tomato DC3000 infection.

### Bacterial Conjugation

DNA sequences controlling extra-chromosomal replication (ori) and transfer (tra) are distinct from one another: i.e., a replication sequence generally does not control plasmid transfer, or vice-versa. Replication and transfer are both complex molecular processes that make use of both plasmid- and host-encoded functions. Bacterial conjugation is the horizontal transmission of genetic information from one bacterium to another. The genetic material transferred may be a plasmid or it may be all or part of a chromosome if a functional origin of transfer is within the chromosome. Bacterial cells possessing a conjugative plasmid contain a surface structure (the sex pilus) that is involved in the coupling of donor and recipient cells, and the transfer of the genetic information. Conjugation involves contact between cells, and the transfer of genetic traits can be mediated by many plasmids. Among all natural transfer mechanisms, conjugation is the most efficient. For example, F plasmid of E. coli, pCFIO plasmid of Enterococcus faecalis and pXO16 plasmid of Bacillus thuringiensis employ different mechanisms for the establishment of mating pairs, the sizes of mating aggregates are different, and they have different host ranges within gram-negative (F) as well as gram-positive (pCFIO and pXO16) bacteria. Their plasmid sizes are also different: 54, 100 and 200 kb, respectively. Remarkably, however, those conjugation systems have very important characteristics in common: they are able to sustain conjugative transfer in liquid medium and high transfer efficiencies are often reached in a very short time. Thus, the conjugative process permits the protection of plasmid DNA against environmental nucleases, and the very efficient delivery of plasmid DNA into a recipient cell. Conjugation functions are naturally plasmid encoded. Numerous conjugative plasmids (and transposons) are known, which can transfer associated genes within one species (narrow host range) or between many species (broad host range). Typically a range of effecincy is observed that is dependant on the incompatibilty group of the plasmid conjuagative system and the conditions and environment where conjugation occurs (Alderliesten, J. B., Duxbury, S. J. N., Zwart, M. P. et al. Effect of donor-recipient relatedness on the plasmid conjugation frequency: a meta-analysis. BMC Microbiol 20, 135 (2020). https://doi.org/10.1186/s12866-020-01825-4). Transmissible plasmids have been reported in numerous Gram-positive genera, including but not limited to pathogenic strains of Streptococcus, Staphylococcus, Bacillus, Clostridium and Nocardia. The early stages of conjugation generally differ in Gram-negative and Gram-positive bacteria. The role of some of the transfer genes in conjugative plasmids from Gram-negative bacteria are to provide pilus-mediated cell-to-cell contact, formation of a conjugation pore and related morphological functions. The pili do not appear to be involved in initiating conjugation in Gram-positive bacteria.

### SUMMARY OF THE INVENTION

The also invention provides the following configurations:-

### In a First Configuration

### In One Aspect:

A method of killing a bacterial target cell, the cell comprising at least one Resistance-Nodulation-Cell Division (RND)-efflux pump, the method comprising contacting the target cell with a carrier bacterial cell, wherein the carrier cell comprises a conjugative plasmid, the plasmid encoding an antibacterial agent that is toxic to the target cell, wherein the carrier cell conjugates to the target cell and the plasmid is transferred into the target cell, wherein the agent is expressed in the target cell and the target cell is killed.

### In Another Aspect:

A method of modifying the genome of a bacterial target cell, the cell comprising at least one Resistance-Nodulation-Cell Division (RND)-efflux pump, the method comprising contacting the target cell with a carrier bacterial cell, wherein the carrier cell comprises a conjugative plasmid, the plasmid encoding an agent that capable of modifying the genome of the target cell, wherein the carrier cell conjugates to the target cell and the plasmid is transferred into the target cell, wherein the agent is expressed in the target cell and the target cell genome is modified.

### In a First Aspect of the First Configuration

The method is a method of increasing the biomass of a plant or part thereof, wherein the method is carried out using a first cell population comprising a plurality of carrier cells that are contacted with a second cell population comprising a plurality of target cells, wherein copies of said plasmid are conjugatively transferred from carrier cells into target cells, whereby some or all of the cells of the second population are killed, wherein the plant comprises said target cells (optionally on leaves and/or stems thereof or comprised by the apoplast of the plant), whereby target cells are killed and said biomass is increased.

### In a Second Aspect of the First Configuration

The method is a method of promoting germination of a plant seed, wherein the method is carried out using a first cell population comprising a plurality of carrier cells that are contacted with a second cell population comprising a plurality of target cells, wherein copies of said plasmid are conjugatively transferred from carrier cells into target cells, whereby some or all of the cells of the second population are killed, wherein the seed comprises said target cells, whereby target cells are killed and germination is promoted.

### In a Third Aspect of the First Configuration

The method is a method of increasing leaf chlorophyll production in a plant, wherein the method is carried out using a first cell population comprising a plurality of carrier cells that are contacted with a second cell population comprising a plurality of target cells, wherein copies of said plasmid are conjugatively transferred from carrier cells into target cells, whereby some or all of the cells of the second population are killed, wherein the plant comprises said target cells (optionally on leaves and/or stems thereof, or comprised by the apoplast of the plant), whereby target cells are killed and chlorophyll is increased in the plant.

### In a Fourth Aspect of the First Configuration

The method is a method of reducing a biofilm comprised by a subject or comprised on a surface, wherein the biofilm comprises target cells, wherein the method is carried out using a first cell population comprising a plurality of carrier cells that are contacted with a second cell population comprising a plurality of target cells, wherein copies of said plasmid are conjugatively transferred from carrier cells into target cells, thereby killing the target cells in the biofilm or reducing the growth or proliferation of target cells, optionally wherein the method is carried out ex vivo or in vitro.

### In a Second Configuration

A carrier bacterial cell for use in a method of killing a bacterial target cell according to the first configuration, wherein the carrier cell comprises a conjugative plasmid, the plasmid encoding an antibacterial agent that is toxic in the target cell, wherein the carrier cell is capable of conjugating to the target cell wherein the plasmid is transferred into the target cell, wherein the agent is expressed in the target cell and the target cell is killed.

### In a Third Configuration

A pharmaceutical composition comprising a plurality of carrier cells of the second configuration for administration to a human or animal subject for killing a plurality of bacterial target cells comprised by the subject, wherein each target cell comprises at least one Resistance-Nodulation-Cell Division (RND)-efflux pump whereby each target cell is an antibiotic resistant cell, wherein plasmids encoding the antibacterial agent are introduced from carrier cells into target cells by conjugation and said antibacterial agent is produced in target cells, whereby target cells are killed and an antibiotic resistant infection of bacterial target cells is treated or prevented in the subject.

### In a Fourth Configuration

A method of treating or preventing a disease or condition in a plant, the method comprising contacting the plant (eg, one or more stems and/or one or more leaves of the plant) with a composition comprising a plurality of carrier cells of the second configuration, wherein the plant comprises target bacterial cells that mediate the disease or condition, wherein each target cell comprises at least one Resistance-Nodulation-Cell Division (RND)-efflux pump, wherein plasmids encoding the antibacterial agent are introduced from carrier cells into target cells by conjugation and said antibacterial agent is produced in target cells, whereby target cells are killed and the disease or condition is treated or prevented.

### In a Fifth Configuration

Use of a carrier cell of the second configuration in the manufacture of a composition, for killing a bacterial target cell ex vivo or wherein the target cell is not comprised by a human or animal (eg, the target cell is comprised by a plant or soil), wherein the target cell comprises at least one Resistance-Nodulation-Cell Division (RND)-efflux pump, wherein the target cell is contacted with the carrier cell and the carrier cell conjugates to the target cell, whereby the plasmid is introduced into the target cell, wherein the antibacterial agent is expressed in the target cell and the target cell is killed.

Optionally, the target bacteria are Pseudomonas bacteria, such as P. syringae or P. aeruginosa bacteria or any other Pseudomonas bacteria disclosed herein. For example, the P. syringae is P. syringae pv. tomato DC3000 and/or the target cells are comprised by a tomato plant, eg, Lycopersicon esculentum cultivar (cv.) Moneymaker.

Optionally, the agent is a guided nuclease system or a component thereof, eg, any such system or component disclosed herein for modifying (eg, cutting) a target nucleic acid sequence comprised by target bacteria.

Optionally, the plant is any plant disclosed herein.

Optionally, the chlorophyll is a chlorophyll a and/or chlorophyll b.

### BRIEF DESCRIPTION OF FIGURES

FIG. 1A-C. The three biological replicates (in the order as labelled) of the protection assay using the plant control GB™ 1 and the plant active GB™ 1. In the biological replicate #2, the CFU/cm² of Pto DC3000 was 0, for the plant active GB™ 1, at the timepoints 48 hrs and 7 days:

FIG. 2A-C: The three biological replicates (in the order as labelled) of the protection assay using the plant control GB™ 2 and the plant active GB™ 2; and

FIG. 3. The Moneymaker tomato plants treated with the plant control GB™ 1 or the plant active GB™ 1 and spray challenged with Pto DC3000. (A and B). Plant control GB™ 1+Pto DC3000 (C and D). Plant active GB™ 1+Pto DC3000.

### DETAILED DESCRIPTION

The invention relates to methods of killing bacterial target cells comprising Resistance-Nodulation-Cell Division (RND)-efflux pumps, as well as carrier cells useful for this purpose wherein the carrier cells comprise a conjugative plasmid encoding an antibacterial-microbial agent that is toxic to target cells. A carrier bacterium is capable of conjugative transfer of plasmid DNA encoding the agent to a target cell.

Reference is made to mBio, 2015 Mar. 24:6(2):e00309-15. doi: 10.1128/mBio.00309-15, “Contribution of resistance-nodulation-cell division efflux systems to antibiotic resistance and biofilm formation in Acinetobacter baumannii”, Eun-Jeong Yoon et al, which studied the expression of RND efflux pumps in A. baumanii. The authors observed that in two types of plasmid transfer, mobilisation and conjugation, high expression of adeABC and adeIJK RND pumps by the recipient bacteria resulted in reproducible reduction of transfer frequencies (see FIG. 4 in Yoon et al). The authors concluded that it thus appears that, if high expression of pumps contributes to multi-drug resistance (MDR) by efflux, it decreases acquisition of foreign DNA by both transformation and conjugation. This suggests that upregulation of expression of RND pumps (such as in response to antibiotics and other antibacterial agents in the environment of the bacteria) can reduce the chances of DNA entry by conjugation. For example, several studies have reported that E. coli biofilms have higher antibiotic resistance than planktonic cells and that expression of several gene-encoded efflux pumps was increased in biofilms (eg, see Ito A, Taniuchi A, May T, Kawata K, Okabe S. Increased antibiotic resistance of Escherichia coli in mature biofilms. Appl Environ Microbiol. 2009:75:4093-100. doi: 10.1128/AEM.02949-08). Hyperexpression of efflux pumps of the RND type in Pseudomonas aeruginosa (e.g., MexAB-OprM), chromosomally encoded by mexAB-oprM, mexCD-oprJ, mexEF-oprN, and mexXY (-oprA), is often detected in clinical isolates and contributes to worrying multi-drug resistance phenotypes. By monitoring the amount of extracellular DNA (eDNA) released by strains overexpressing pmt, Sahu et al (“Characterization of eDNA from the clinical strain Acinetobacter baumannii AIIMS 7 and its role in biofilm formation”, Sahu P K et al, Scientific World Journal. 2012; 2012:973436) proposed the involvement of the Pmt efflux pump (a MFS pump) in nucleic acid transport. Since DNA and RNA are well-known scaffolding components of the biofilm matrix, the authors have inferred that an increase in eDNA supports a more abundant development of the bacterial biofilm.

In view of the art, such as these teachings, it is surprising that the inventors could successfully deliver a plasmid-borne antibacterial agent using conjugation into bacteria comprising RND efflux pumps (see Examples). Targeted killing of the desired bacteria was surprisingly and advantageously achieved. The invention will, for example, be particularly useful for targeting bacteria in biofilms.

In particular, very high rates of targt cell killing were surprisingly observed using plasmid conjugation and CRISPR/Cas killing as shown in the Examples. Killing of more than 90% of target cells was reproducibly and advantageously achieved despite the presence of RND efflux pumps in target strains.

In addition, we could surprisingly achieve maintenance of bacteriocidal effect on surfaces (as exemplified by leaf surfaces, Example 1).

Thus, there is provided:-

A method of killing a bacterial target cell, the cell comprising at least one Resistance-Nodulation-Cell Division (RND)-efflux pump, the method comprising contacting the target cell with a carrier bacterial cell, wherein the carrier cell comprises a conjugative plasmid, the plasmid encoding an antibacterial agent that is toxic to the target cell, wherein the carrier cell conjugates to the target cell and the plasmid is transferred into the target cell, wherein the agent is expressed in the target cell and the target cell is killed.

In an alternative, instead of bacterial cells, the carrier and target cells may be archaea.

In another alternative, instead of killing the target cell, the method modifies the genome of the cell, eg, modifies a chromosome or episome (eg, a plasmid) of the cell. Modification may be cutting of the chromosome or episome, for example, such as where the agent is a guided nuclease. An example of such a nuclease is a Cas, megagunclease, TALEN or zinc finger nuclease. Thus, there is also provided:-

A method of modifying the genome of a bacterial target cell, the cell comprising at least one Resistance-Nodulation-Cell Division (RND)-efflux pump, the method comprising contacting the target cell with a carrier bacterial cell, wherein the carrier cell comprises a conjugative plasmid, the plasmid encoding an agent that capable of modifying the genome of the target cell, wherein the carrier cell conjugates to the target cell and the plasmid is transferred into the target cell, wherein the agent is expressed in the target cell and the target cell genome is modified.

Optionally, the target cell is resistant to one or more antibiotics. The target cell may comprise an efflux pump may mediates antibiotic resistance in the target cell. The target cell may comprise an efflux pump may mediates resistance of the target cell to one or more antibacterial agents.

The carrier cell and target cell may be cells of the same order, family or genus, such as shown in the Examples.

Preferably, the agent comprises a CRISPR/Cas system or component thereof. The agent may be a crRNA or guide RNA that guides a Cas nuclease in the target cell to a target protospacer sequence, wherein the Cas cuts the target sequence and the target cell is killed. For example, the plasmid may encode a plurality of different crRNAs or guide RNAs, such as a first cRNA or gRNA that comprises a spacer sequence that is capable of guiding a Cas in the target cell to a first protospacer sequence and a second cRNA or gRNA that comprises a spacer sequence that is capable of guiding a Cas in the target cell to a second protospacer sequence wherein the protospacer sequences are different (eg, different chromosomal sequences of the target cell). Each protospacer may be comprised by an essential gene, virulence gene or antibiotic resistance gene of the target cell genome. Each protospacer sequence may be from 10 to 60 nucleotides in length, eg, 15 to 50, 15 to 40, 15 to 30 or 15 to 20 nucleotides in length. The target sequence may be a chromosomal sequence of the target cell. The target sequence may be an episomal sequence of the target cell. The plasmid may encode a or said Cas nuclease, optionally a Cas9, Cas3 or Cpf1.

In an example, the target cell comprises an RND efflux pump of a strain selected from
(i) Azotobacter chroococcum NCIMB 8003, Azotobacter chroococcum strain B3, Azotobacter salinestris strain KACC 13899, Burkholderia ambifaria MC40-6, Burkholderia cenocepacia AU 1054 chromosome 1, Burkholderia cenocepacia HI2424 chromosome 3, Burkholderia cenocepacia MC0-3, Burkholderia cenocepacia strain CR318 chromosome 3, Burkholderia cenocepacia strain FDAARGOS_720, Burkholderia lata strain A05, Burkholderia pyrrocinia strain mHSR5, Cupriavidus basilensis strain 4G11, Cupriavidus necator N-1 plasmid pBB1, Cupriavidus taiwanensis STM 3679, Lysobacter gummosus strain 3.2.11, Paraburkholderia sprentiae WSM5005, Paraburkholderia terricola strain mHS1, Ralstonia pseudosolanacearum strain CRMRs218, Ralstonia solanaccarum strain UA-1591, Variovorax paradoxus S110, Variovorax sp. PBL-H6, Xanthomonas arboricola pv. juglandis strain Xaj 417, Xanthomonas arboricola pv. pruni strain 15-088, Xanthomonas arboricola strain 17, Xanthomonas axonopodis pv. dieffenbachiae LMG 695, Xanthomonas axonopodis pv. phascoli strain ISO18C8, Xanthomonas axonopodis pv. phaseoli strain ISO98C12, Xanthomonas campestris pv. campestris MAFF302021, Xanthomonas citri pv. glycines strain 2098, Xanthomonas euvesicatoria strain LMG930, Xanthomonas perforans strain LH3 and Xanthomonas sp. ISO98C4, which strains have NCBI Accession Numbers respectively of CP010415.1, CP011835.1, CP045302.1, CP001027.1, CP000378.1, CP000460.1, CP000960.1, CP017240.1, CP050980.1, CP024945.1, CP024903.1, CP010537.1, CP002879.1, LT984803.1, CP011131.1, CP017561.1, CP024941.1. CP021764.1, CP034195.1, CP001636.1, LR594659.1, CP012251.1, CP044334.1, CP011256.1, CP014347.1, CP012063.1, CP012057.1, AP019684.1, CP041965.1, CP018467.1. CP018475.1 and CP012060.1, or an orthologue or homologue of such a pump;
(ii) Pseudomonas aeruginosa strain: IOMTU 133, Pseudomonas aeruginosa DSM 50071, Pseudomonas aeruginosa genome assembly NCTC10332, Pseudomonas aeruginosa isolate B10W, Pseudomonas aeruginosa isolate PA14Or, Pseudomonas aeruginosa NCGM2.S1, Pseudomonas aeruginosa PAK, Pseudomonas aeruginosa strain 243931, Pseudomonas aeruginosa strain 24Pae 112, Pseudomonas aeruginosa strain 268, Pseudomonas aeruginosa strain 60503, Pseudomonas aeruginosa strain AR_0095, Pseudomonas aeruginosa strain AR_0353, Pseudomonas aeruginosa strain AR_0354, Pseudomonas aeruginosa strain AR_455, Pseudomonas aeruginosa strain BAMCPA07-48, Pseudomonas aeruginosa strain CCUG 51971, Pseudomonas aeruginosa strain E90, Pseudomonas aeruginosa strain FDAARGOS_571, Pseudomonas aeruginosa strain GIMC5002:PAT-169, Pseudomonas aeruginosa strain H26023, Pseudomonas aeruginosa strain L10, Pseudomonas aeruginosa strain M1608, Pseudomonas aeruginosa strain M37351, Pseudomonas acruginosa strain MRSN12280, Pseudomonas aeruginosa strain NCTC13715, Pseudomonas aeruginosa strain Pa58, Pseudomonas aeruginosa strain PABLO48, Pseudomonas aeruginosa strain PAK, Pseudomonas aeruginosa strain PASGNDM345, Pseudomonas acruginosa strain PASGNDM699, Pseudomonas aeruginosa strain PA-VAP-3, Pseudomonas aeruginosa strain PB368, Pseudomonas aeruginosa strain PB369, Pseudomonas aeruginosa strain S04 90, Pseudomonas aeruginosa strain ST773,Pseudomonas aeruginosa strain T2436, Pseudomonas aeruginosa strain W60856, Pseudomonas aeruginosa strain WPB099, Pseudomonas aeruginosa strain WPB100, Pseudomonas aeruginosa strain WPB101, Pseudomonas aeruginosa UCBPP-PA14, Pseudomonas aeruginosa UCBPP-PA14, Pseudomonas aeruginosa VRFPA04, Pseudomonas amygdali pv. lachrymans str. M301315, Pseudomonas amygdali pv. lachrymans strain NM002, Pseudomonas amygdali pv. morsprunorum strain R15244, Pseudomonas amygdali pv. tabaci str. ATCC 11528, Pseudomonas avellanae strain R2leaf, Pseudomonas coronafaciens pv. coronafaciens strain B19001, Pseudomonas coronafaciens pv. oryzae str. 1_6, Pseudomonas coronafaciens strain X-1, Pseudomonas otitidis MrB4, Pseudomonas salegens strain CECT 8338, Pseudomonas savastanoi pv. phascolicola 1448A, Pseudomonas savastanoi pv. savastanoi NCPPB 3335, Pseudomonas sp. KBS0707, Pseudomonas sp. LPHI, Pseudomonas syringae CC1557, Pseudomonas syringae group genomosp. 3 isolate CFBP6411, Pseudomonas syringae isolate CFBP3840, Pseudomonas syringae pv. actinidiae ICMP 18708, Pseudomonas syringae pv. actinidiac ICMP 18884, Pseudomonas syringae pv. actinidiae ICMP 9853, Pseudomonas syringae pv. actinidiae str. Shaanxi_M228, Pseudomonas syringae pv. actinidiac strain CRAFRU 12.29, Pseudomonas syringae pv. actinidiae strain CRAFRU 14.08, Pseudomonas syringae pv. actinidiae strain MAFF212063, Pseudomonas syringae pv. actinidiae strain NZ-45, Pseudomonas syringae pv. actinidiae strain NZ-47, Pseudomonas syringae pv. actinidiae strain P155, Pseudomonas syringae pv. avii isolate CFBP3846, Pseudomonas syringae pv. cerasicola isolate CFBP6109, Pseudomonas syringae pv. maculicola str. ES4326, Pseudomonas syringae pv. tomato str. DC3000, Pseudomonas syringae pv. tomato strain B13-200, Pseudomonas syringae pv. tomato strain delta IV/IX, Pseudomonas syringae pv. tomato strain delta VI, Pseudomonas syringae pv. tomato strain delta X, Pseudomonas syringae strain CFBP 2116 and Pseudomonas syringae strain Ps25, which strains have NCBI Accession Numbers respectively of AP017302.1, CP012001.1, LN831024.1, CP017969.1, LT608330.1. AP012280.1, CP020659.1, CP041772.1, CP029605.1, CP032761.1, CP041774.1, CP027538.1, CP027172.1, CP027171.1, CP030328.1, CP015377.1, CP043328.1, CP044006.1, CP033833.1, CP043549.1, CP033685.1, CP019338.1, CP008862.2, CP008863.1, CP028162.1, LR134330.1, CP021775.1, CP039293.1, LR657304.1, CP020703.1, CP020704.1, CP028330.1, CP025050.1, CP025049.1, CP011369.1, CP041945.1, CP039988.1, CP008864.2, CP031878.1, CP031877.1, CP031876.1, CP034244.1, CP000438.1, CP008739.2, CP031225.1, CP020351.1, CP026558.1, CP042804.1, CP026562.1, CP046441.1, CP046035.1, CP050260.1, AP022642.1, LT629787.1, CP000058.1, CP008742.1, CP041754.1, CP017290.1, CP007014.1, LT963408.1, LT963409.1, CP012179.1, CP011972.2, CP018202.1, CP032631.1, CP019730.1, CP019732.1, CP024712.1, CP017007.1, CP017009.1, CP032871.1, LT963402.1, LT963391.1, CP047260.1, AE016853.1, CP019871.1, CP047072.1, CP047071.1, CP047073.1, LT985192.1 and CP034558.1, or an orthologue or homologue of such a pump;
(iii) Stenotrophomonas rhizophila strain GA1, Enterococcus faecalis strain V583 and Paucimonas lemoignei strain NCTC10937, which strains have NCBI Accession Numbers respectively CP031729.1, CP022312.1 and LS483371.1, or an orthologue or homologue of such a pump: or
(iv) Pseudomonas amygdali pv. lachrymans strain NM002, Pseudomonas amygdali pv. morsprunorum strain R15244, Pseudomonas amygdali pv. tabaci str. ATCC 11528, Pseudomonas asturiensis strain CC1524, Pseudomonas avellanae strain R2, Pseudomonas cerasi isolate PL963, Pseudomonas chlororaphis strain PCL1606, Pseudomonas chlororaphis subsp. aurantiaca strain JD37, Pseudomonas chlororaphis subsp. aureofaciens strain ChPhzTR36, Pseudomonas chlororaphis subsp. chlororaphis strain DSM 50083, Pseudomonas chlororaphis subsp. piscium strain DSM 21509, Pseudomonas cichorii JBC1, Pseudomonas coronafaciens pv. coronafaciens strain B19001, Pseudomonas putida GB-1 chromosome, Pseudomonas savastanoi pv. phaseolicola 1448A, Pseudomonas sp. 09C 129, Pseudomonas syringae CC1557, Pseudomonas syringae pv. actinidiae ICMP 18708, Pseudomonas syringae pv. cerasicola isolate CFBP6109, Pseudomonas syringae pv. lapsa strain ATCC 10859, Pseudomonas syringae pv. maculicola str. ES4326, Pseudomonas syringae pv. pisi str. PP1, Pseudomonas syringae pv. syringae B301D, Pseudomonas syringae pv. syringae B301D, Pseudomonas syringae UMAF0158 and Pseudomonas viridiflava strain CFBP 1590, which strains have NCBI Accession Numbers respectively of CP020351.1, CP026558.1, CP042804.1, CP047265.1, CP026562.1, LT963395.1, CP011110.1, CP009290.1, CP027721.1, CP027712.1, CP027707.1, CP007039.1, CP046441.1, CP000926.1, CP000058.1, CP025261.1, CP007014.1, CP012179.1, LT963391.1, CP013183.1, CP047260.1, CP034078.1, CP005969.1, AE016853.1, CP005970.1 and LT855380.1, or an orthologue or homologue of such a pump.

The RND efflux pump of the target cell may comprise a protein produced by any of these strains. The target cell may be a cell of any of these strains. Any NCBI database and related Accession numbers are, for example, those publicly available on 27 Apr. 2020.

The efflux pump may comprise a protein encoded by a Pseudomonas syringae gene selected from PSPTO_0820, PSPTO_4977, PSPTO_02375, PSPTO_1308, PSPTO_2592, PSPTO_2755, PSPTO_3100, PSPTO_3302, PSPTO_430 or PSPTO_5191, or an orthologue or homologue thereof.

The efflux pump may comprise a protein encoded by
(a) Pseudomonas syringae PSPTO_0820 or PSPTO_4977 gene or an orthologue or homologue thereof; or
(a) A nucleotide sequence selected from SEQ ID NO: 1 and 3, or a nucleotide sequence that is at least 70% identical (eg, at least 80, 85, 90, 95, 96, 97, 98 or 99% identical) to SEQ ID NO: 1 or 3.

The efflux pump may comprise a protein comprising the amino acid sequence of SEQ ID NO: 2 or 4, or an amino acid sequence that is at least 70% identical (eg, at least 80, 85, 90, 95, 96, 97, 98 or 99% identical) to SEQ ID NO: 2 or 4.

The efflux pump may be a Mex efflux pump (optionally a MexAB-OprM efflux pump, MexCD-OprJ efflux pump, MexEF-OprN efflux pump or MexXY efflux pump), AdeABC efflux pump, AcrAD-TolC efflux pump, AcrAB-TolC efflux pump, AcrABZ-TolC efflux pump, AcrA efflux pump, ArcB efflux pump, AcrC efflux pump, AcrD efflux pump, AcrAB efflux pump, AcrEF efflux pump, AcrF efflux pump, CmeABC efflux pump, VexB efflux pump, VexD efflux pump, VexK efflux pump, ade ABC efflux pump, adeIJK efflux pump, MdsABC efflux pump or MdtABC efflux pump. Preferably, the pump is an AcrAD-TolC efflux pump.

The carrier cell may be a Pseudomonas cell, optionally a P. fluorescens cell. Optionally, the carrier and target cells are cells of the same genus or species, optionally both are Pseudomonas cells. For example, the target cell is a P. syringae or aeruginosa cell and the carrier is a Pseudomonas (eg, P. fluorescens) cell. This is demonstrated in the Examples.

Preferably, the carrier cells are of a strain or species that is not pathogenic to an organism (eg, a plant, animal or human) that comprises the target cells. The carrier cells may be of a strain or species that is symbiotic or probiotic to an organism (eg, a plant, animal or human) that comprises the target cells, eg, probiotic or symbiotic in the gut of the organism.

In an example, the carrier cell comprises a Chitinase class I exoenzyme and/or the carrier cell genome encodes a Chitinase class I exoenzyme. Optionally, the carrier cell in this example is a Pseudomonas, eg, P. fluorescens, cell.

In an example, the carrier cell comprises a pep1 gene. Optionally, the carrier cell in this example is a Pseudomonas, eg, P. fluorescens, cell.

In an example, the carrier cell is a motile bacterial cell. Optionally, the carrier cell in this example is a Pseudomonas, eg, P. fluorescens, cell.

For example, each target cell is a lag phase cell, exponential phase cell or a stationary phase cell. For example, each carrier cell is a lag phase cell, exponential phase cell or a stationary phase cell.

Preferably, the target cell is a Pseudomonas (optionally a P. fluorescens or P. aeruginosa) cell, Erwinia (optionally E. carotovora), Xanthomonas, Agrobcaterium, Burkholdi, Clavibacterium, Enterobacteria, Pantoae, Pectobacterium (eg, P. atrosepticum), Rhizobium, Streptomyces (eg, S scabies), Xylella (eg, X fastidiosa), Candidatus (eg, C liberibacter), Phytoplasma, Ralstonia (eg, R. solanacearum), or Dickeya (eg, D dadantii) cell.

Each target cell (eg, the plurality of target cells) may be a cell of a genus or species disclosed in Table 1 or 2. Each target cell (eg, the plurality of target cells) may be comprised by a plant or a plant environment (such as soil) and selected from a genus or species disclosed in Table 1.

The method may be carried out in vitro or ex vivo.

The target cell may be comprised by
(a) a plant microbiome (eg, a microbiome of any plant part disclosed herein),
(b) an animal or human microbiome (eg, a microbiome of any human or animal organ or tissue or part disclosed herein,: or
(c) a soil, manure, food or beverage microbiome.

For example, the cell is comprised by a plant leaf, stem, root, seed, bulb, flower or fruit microbiome.

Optionally, a microbiome herein is a gut, lung, kidney, urethral, bladder, blood, vaginal, eye, ear, nose, penile, bowel, liver, heart, tongue, hair or skin microbiome.

For example, the target cell is a cell of a species found in soil.

The method may be carried out using a first cell population comprising a plurality of carrier cells that are contacted with a second cell population comprising a plurality of target cells, wherein copies of said plasmid are conjugatively transferred from carrier cells into target cells, whereby some or all of the cells of the second population are killed.

The method may reduce the number of target cells of said plurality at least 10⁵, 10⁶ or 10⁷-fold, eg, between 10⁵ and 10⁷-fold, or between 10⁵ and 10⁸-fold or between 10⁵ and 10⁹-fold. The skilled person will be familiar with determining fold-killing or reduction in cells, eg, using a cell sample that is representative of a microbiome or cell population. For example, the extent of killing or reduction is determined using a cell sample, eg, a sample obtained from a subject to which the carrier cells of the invention have been administered, or an environmental sample (eg, aqueous, water or soil sample) obtained from an environment (eg, a water source, waterway or field) that has been contacted with the carrier cells of the invention. For example, the method reduces the number of target cells of said plurality at least 10⁵, 10⁶ or 10⁷-fold and optionally the plurality comprises at least 100,000; 1,000,000: or 10,000,000 target cells respectively. Optionally, the plurality of target cells is comprised by a cell population, wherein at least 5, 6 or 7 log 10 of cells of the population are killed by the method, and optionally the plurality comprises at least 100,000; 1,000,000; or 10,000,000 target cells respectively.

Optionally, the method kills at least 99%, 99.9%, 99.99%, 99.999%, 99.9999% or 99.99999% cells of said plurality of target cells.

In an example, the method is carried out on a population (or said plurality) of said target cells and the method kills dits all (or essentially all) of the cells of said population (or said plurality). In an example, the method is carried out on a population (or said plurality) of said target cells and the method kills 100% (or about 100%) of the cells of said population (or plurality).

Preferably, at least 90, 91, 92, 93, 94, 95, 96, 97, 98 or 99% of the target cells are killed. This is surprisingly reproducibly demonstrated in the Examples (using conjugative delivery of components of a CRISPR/Cas antibacterial system to target cells).

### In an Aspect:-

The method is a method of increasing the biomass of a plant or part thereof, wherein the method is carried out using a first cell population comprising a plurality of carrier cells that are contacted with a second cell population comprising a plurality of target cells, wherein copies of said plasmid are conjugatively transferred from carrier cells into target cells, whereby some or all of the cells of the second population are killed, wherein the plant comprises said target cells (optionally on leaves and/or stems thereof or comprised by the apoplast of the plant), whereby target cells are killed and said biomass is increased.

Optionally, the target cells are Pseudomonas (eg, P. syringae) cells, eg, wherein the cells are comprised by a crop plant, such as a tomoto plant.

For example, leaf, fruit, ear, seed, grain, head, pod, stem, trunk, tuber and/or root biomass is increased. For example, leaf or fruit dry biomass, leaf or fruit wet biomass or number of flowers is increased. For example, average biomass or number is increased over a plurality of plants on which the method of the invention has been practised.

An increase in biomass (eg, average biomass or number) may be an increase by at least 5, 10, 15, 20, 25, 30, 40, or 50% compared to the biomass of plant(s) that have not been exposed to the carrier bacteria, but which comprise the target bacteria. Increases in plant biomass may be determined by measuring the weight of harvested material (eg, fruit, grain, cane, leaves, tubers, nuts or seeds) per area harvested and comparing the measurement of harvested material from plants that have been treated per the invention versus the same area of harvestsed material from plants of the same species and strain grown that have not been treated per the invention, where all plants are grown under the same conditions, eg, in the same field. In some systems units of volume, such as bushels, are used instead of units of weight.

### In an Aspect:-

The method is a method of promoting germination of a plant seed, wherein the method is carried out using a first cell population comprising a plurality of carrier cells that are contacted with a second cell population comprising a plurality of target cells, wherein copies of said plasmid are conjugatively transferred from carrier cells into target cells, whereby some or all of the cells of the second population are killed, wherein the seed comprises said target cells, whereby target cells are killed and germination is promoted.

Promoting germination may be decreasing the time to onset of germination and/or decreasing the duration of germination. Promoting germination may be increasing the percentage (eg, by at least 5, 10, 15 or 20%) of germination of seeds comprised by a plurality of seeds that are exposed to the carrier cells in the method.

Each seed may comprise target cells on the seed surface.

An increase in germination (eg, average germination) in a plurality of seeds exposed to the carrier cells in the method may be obtained, which is an increase by at least 5, 10, 15, 20, 25, 30, 40, or 50% compared to the germination of seeds that have not been exposed to the carrier cells, but which seeds comprise the target bacteria.

The method may be useful for treating pre-emergent seedlings have pathogens present which stop successful germination. Thus, an Aspect provides:-

The method is a method of promoting growth of a plant seedling, wherein the method is carried out using a first cell population comprising a plurality of carrier cells that are contacted with a second cell population comprising a plurality of target cells, wherein copies of said plasmid are conjugatively transferred from carrier cells into target cells, whereby some or all of the cells of the second population are killed, wherein the seedling comprises said target cells, whereby target cells are killed and seedling growth is promoted.

Each seedling may comprise target cells on leaves and/or stems of the seedling.

An increase in growth (eg, average growth) in a plurality of seedlings exposed to the carrier cells in the method may be obtained, which is an increase by at least 5, 10, 15, 20, 25, 30, 40, or 50% compared to the growth of seedlings that have not been exposed to the carrier cells, but which seedlings comprise the target bacteria.

### An Aspect:-

The method is a method of increasing leaf chlorophyll (eg, chlorophyll a and/or b) production in a plant, wherein the method is carried out using a first cell population comprising a plurality of carrier cells that are contacted with a second cell population comprising a plurality of target cells, wherein copies of said plasmid are conjugatively transferred from carrier cells into target cells, whereby some or all of the cells of the second population are killed, wherein the plant comprises said target cells (optionally on leaves and/or stems thereof, or comprised by the apoplast of the plant), whereby target cells are killed and chlorophyll is increased in the plant. Chlorophyll measurement may be measured, for example, by spectrophotometry, high performance liquid chromatography (HPLC) or fluorometry.

The method is a method of increasing the amount of chlorophyll (eg, chlorophyll a and/or b) in a plant, wherein the method is carried out using a first cell population comprising a plurality of carrier cells that are contacted with a second cell population comprising a plurality of target cells, wherein copies of said plasmid are conjugatively transferred from carrier cells into target cells, whereby some or all of the cells of the second population are killed, wherein the plant comprises said target cells (optionally on leaves and/or stems thereof, or comprised by the apoplast of the plant), whereby target cells are killed and chlorophyll is increased in the plant.

### An Aspect:-

The method is a method of reducing a biofilm comprised by a subject or comprised on a surface, wherein the biofilm comprises target cells, wherein the method is carried out using a first cell population comprising a plurality of carrier cells that are contacted with a second cell population comprising a plurality of target cells, wherein copies of said plasmid are conjugatively transferred from carrier cells into target cells, thereby killing the target cells in the biofilm or reducing the growth or proliferation of target cells, optionally wherein the method is carried out ex vivo or in vitro.

The subject may be a human or animal, optionally wherein the surface is a lung surface.

The subject may be a plant, optionally wherein the biofilm is comprised by a leaf, trunk, root or stem of the plant.

The surface may be comprised by a domestic or industrial apparatus or container, eg, a fermentation vessel.

### There is Further Provided:-

A carrier bacterial cell for use in a method of killing a bacterial target cell according to the invention, wherein the carrier cell comprises a conjugative plasmid, the plasmid encoding an antibacterial agent that is toxic in the target cell, wherein the carrier cell is capable of conjugating to the target cell wherein the plasmid is transferred into the target cell, wherein the agent is expressed in the target cell and the target cell is killed.

The carrier cell may be any carrier cell or carrier cell disclosed herein. The target cell may be any carrier cell or target cell disclosed herein.

### There is Provided:-

A pharmaceutical composition comprising a plurality of carrier cells of the invention for administration to a human or animal subject for killing a plurality of bacterial target cells comprised by the subject, wherein each target cell comprises at least one Resistance-Nodulation-Cell Division (RND)-efflux pump whereby each target cell is an antibiotic resistant cell, wherein plasmids encoding the antibacterial agent are introduced from carrier cells into target cells by conjugation and said antibacterial agent is produced in target cells, whereby target cells are killed and an antibiotic resistant infection of bacterial target cells is treated or prevented in the subject.

Preferably, at least 90, 91, 92, 93, 94, 95, 96, 97, 98 or 99% of the target cells are killed. This is surprisingly reproducibly demonstrated in the Examples (using conjugative delivery of components of a CRISPR/Cas antibacterial system to target cells).

The plurality of target cells may comprise at least 107, 108, 109, 1010, 1011 or 1012 target cells. For example, the plurality of target cells is comprised by a gut, blood, lung, oral cavity, liver, kidney, bladder, urethra or skin microbiota of the subject.

### There is Provided:-

A method of treating or preventing a disease or condition in a plant, the method comprising contacting the plant (eg, one or more stems and/or one or more leaves of the plant, or the plant apoplast) with a composition comprising a plurality of carrier cells of the invention, wherein the plant comprises target bacterial cells that mediate the disease or condition, wherein each target cell comprises at least one Resistance-Nodulation-Cell Division (RND)-efflux pump, wherein plasmids encoding the antibacterial agent are introduced from carrier cells into target cells by conjugation and said antibacterial agent is produced in target cells, whereby target cells are killed and the disease or condition is treated or prevented.

Use of a plurality of carrier cells of the invention in the manufacture of a composition for administration to a plant or environment (eg, soil), for killing bacterial target cells comprised by the plant or environment, wherein the target cells comprise at least one Resistance-Nodulation-Cell Division (RND)-efflux pump, wherein the target cells are contacted with the carrier cells and the plasmids comprising the anti-microbial agent are transferred into the target cells, wherein the agent is expressed in the target cells and the target cells are killed.

Use of a carrier cell of the invention in the manufacture of a composition, for killing a bacterial target cell ex vivo or wherein the target cell is not comprised by a human or animal (eg, the target cell is comprised by a plant or soil), wherein the target cell comprises at least one Resistance-Nodulation-Cell Division (RND)-efflux pump, wherein the target cell is contacted with the carrier cell and the carrier cell conjugates to the target cell, whereby the plasmid is introduced into the target cell, wherein the antibacterial agent is expressed in the target cell and the target cell is killed.

Optionally, the use comprises using a plurality of said carrier cells to kill a plurality of said target cells, wherein the target cells are comprised by a plant or plant environment (eg, soil) and the killing
a) increases (or is for increasing) the biomass of the plant or part thereof (eg, leaf, fruit, ear, seed, grain, head, pod, stem, trunk, tuber and/or root biomass is increased);
b) promotes (or is for promoting) germination of one or more seeds of the plant;
c) increases (or is for increasing) the amount of leaf chlorophyll of the plant; and/or
d) reduces (or is for reducing) a biofilm comprised by the plant, wherein the biofilm comprises target cells (eg, Pseudomonas cells).

Optionally, the target cell or plurality of target cells is in an environment, eg, soil, or in an environment for growing plants.

### Example Target Cells

For example, each target cell is a gram-positive bacterial cell (eg, a Staphylococcus (such as S. aureus, eg, methicillin-resistant Staphylococcus aureus (MRSA)), Streptococcus pneumoniae, Clostridium difficile, Enterococcus spp. or Listeria monocytogenes cell). For example, each target cell is a gram-negative bacterial cell (eg, a Acinetobacter baumannii, Escherichia coli, Klebsiella pneumoniae, Stenotrophomonas maltophilia, Campylobacter jejuni, Pseudomonas aeruginosa, Neisseria gonorrhoeae, Vibrio cholerae or Salmonella spp. Cell) For example, each target cell is a cell of a genus or species disclosed in Table 1 herein, Table 2 herein. Reference is made to Journal of Plant Pathology (2010), 92 (3), 551-592 Edizioni ETS Pisa, 2010 551, LETTER TO THE EDITOR, “COMPREHENSIVE LIST OF NAMES OF PLANT PATHOGENIC BACTERIA, 1980-2007”, C.T. Bull et al, the disclosure of which is incorporated herein by reference to provide examples of bacterial genera, species and strains of importance to plants and which may be genera, species and strains of target cells of the invention. Examples are disclosed in Table 1 herein.

For example, each target cell is resistant to a fluoroquinolone, β-lactam (eg, methicillin), tetracycline or linezolid antibiotic. For example, each target cell is resistant to vancomycin, eg, wherein the cell is a vancomycin-resistant Enterococcus cell.

For example, each target cell is an Azotobacter, Burkholderia, Cupriavidus, Enterococcus, Lysobacter, Paucimonas, Paraburkholderia, Ralstonia, Stenotrophomonas, Variovorax, Xanthomonas or Pseudomonas cell.

For example, each target cell is an E. coli cell, eg, wherein the efflux pump protein is encoded by TolC or an orthologue or homologue of such a pump protein. For example, each target cell is Klebsiella cell (such as K. pneumoniae cell), eg, wherein the efflux pump protein is selected from KexC, KexD, KexE, KexF, KexEF, AcrA, AcrB, AcrAB, OqxA, OqxB, OqxAB, EefA, EefB, EefC and EefABC or an orthologue or homologue of such a pump protein.

For example, each target cell is an Azotobacter, Burkholderia, Cupriavidus, Lysobacter, Paraburkholderia, Ralstonia, Variovorax, Xanthomonas or Pseudomonas cell.

For example, each target cell is a cell of a Pseudomonas species, optionally wherein the species is selected from Pseudomonas aeruginosa Pseudomonas amygdali, Pseudomonas asturiensis, Pseudomonas avellanae, Pseudomonas cerasi, Pseudomonas chlororaphis, Pseudomonas cichorii, Pseudomonas coronafaciens, Pseudomonas otitidis, Pseudomonas putida, Pseudomonas salegens Pseudomonas savastanoi, Pseudomonas syringae and Pseudomonas viridiflava.

For example, each target cell is a cell of a species selected from Azotobacter chroococcum, Azotobacter salinestris, Burkholderia ambifaria, Burkholderia cenocepacia, Burkholderia lata, Burkholderia pyrrocinia, Cupriavidus basilensis, Cupriavidus necator, Cupriavidus taiwanensis, Lysobacter gummosus, Paraburkholderia sprentiae, Paraburkholderia terricola, Ralstonia pseudosolanacearum, Ralstonia solanacearum, Variovorax paradoxus, Xanthomonas arboricola, Xanthomonas axonopodis, Xanthomonas campestris Xanthomonas citri, Xanthomonas euvesicatoria and Xanthomonas perforans.

For example, each target cell is a Stenotrophomonas, Enterococcus, Paucimonas or Pseudomonas cell.

For example, each target cell is a cell of a Pseudomonas species, optionally wherein the species is selected from Pseudomonas amygdali, Pseudomonas asturiensis, Pseudomonas avellanae, Pseudomonas cerasi, Pseudomonas chlororaphis, Pseudomonas cichorii, Pseudomonas coronafaciens, Pseudomonas putida, Pseudomonas savastanoi, Pseudomonas syringae and Pseudomonas viridiflava.

For example, each target cell is a cell of a species selected from Stenotrophomonas rhizophila, Enterococcus faecalis, Paucimonas lemoignei, Pseudomonas amygdali, Pseudomonas asturiensis, Pseudomonas avellanae, Pseudomonas cerasi, Pseudomonas chlororaphis, Pseudomonas cichorii, Pseudomonas coronafaciens, Pseudomonas putida, Pseudomonas savastanoi, Pseudomonas syringae and Pseudomonas viridiflava.

Optionally, the efflux pump comprises a protein
(a) encoded by Pseudomonas syringae PSPTO_0820 or PSPTO_4977 gene or an orthologue or homologue thereof; or
(b) produced by a strain disclosed herein.

Optionally, the genome of the target cell comprises (i) a P. syringae PSPTO_0820 gene or an orthologue or homologue thereof; and (ii) a P. syringae PSPTO_4977 gene or an orthologue or homologue thereof.

Optionally, the efflux pump is a MexAB-OprM efflux pump, eg, P. syringae MexAB-OprM efflux pump. Optionally, the efflux pump protein is a protein of such a pump.

Optionally, the efflux pump is an AdeABC efflux pump, eg, A. baumannii AdeABC efflux pump. Optionally, the efflux pump protein is a protein of such a pump.

Optionally, the efflux pump protein is encoded by Pseudomonas syringae gene PSPTO_0820, PSPTO_4977, PSPTO_02375, PSPTO_1308, PSPTO_2592, PSPTO_2755, PSPTO_3100, PSPTO_3302, PSPTO_430 or PSPTO_5191, or an orthologue or homologue thereof. See Table 7 for the role of the products of such genes in P. syringae. The orthologoue or homologue may be from a different genus or species (ie not Pseudomonas or P. syringae).

Optionally, the efflux pump protein is a Pseudomonas syringae AcrB, D or F family protein or a homologue or orthologue thereof. Optionally, the efflux pump protein is a Pseudomonas syringae cation efflux protein or a homologue or orthologue thereof. Optionally, the efflux pump protein is a Pseudomonas syringae isothyocyanate protein or a homologue or orthologue thereof. Optionally, the efflux pump protein is a Pseudomonas syringae TpsC transporter protein or a homologue or orthologue thereof. Optionally, the efflux pump protein is a Pseudomonas syringae SaxG protein or a homologue or orthologue thereof. Optionally, the efflux pump protein is a Pseudomonas aeruginosa MexB, D or F protein or a homologue or orthologue thereof. Optionally, the efflux pump is a Pseudomonas aeruginosa MexAB-OprM, MexCD-OprJ, MexEF-OprN or MexXY pump or a homologue or orthologue thereof. Optionally, the efflux pump protein is a Pseudomonas aeruginosa MexAB-OprM, MexCD-OprJ, MexEF-OprN or MexXY pump protein or a homologue or orthologue thereof. The orthologoue or homologue may be from a different genus or species (ie not Pseudomonas or P. syringae).

Optionally, the efflux pump protein is a protein of a Mex efflux pump. The Mex protein may be a protein that is a surface exposed protein on the target bacteria. In some embodiments, the Mex protein is selected from the group consisting of OprM, MexA, MexB, MexX, and MexY.

Optionally, the efflux pump protein is a bacterial TolC protein (eg, a Pseudomonas or E. coli TolC protein) or a homologue or orthologue thereof.

In an example, each target cell is comprised by a plant microbiome. In an example, each target cell is comprised by an environment microbiome, eg, a water or waterway (eg, river, pond, lake or sea) microbiome. In an example, each target cell is comprised by a soil microbiome. In an example, each target cell is comprised by an animal (ie, non-human animal) microbiome. In an example, each target cell is comprised by a human microbiome (eg, a lung, kidney, GI tract, gut, blood, oral, nasal or liver microbiome).

### PSPTO_0820, Orthologues & Homologues

A PSPTO_0820 gene orthologue or homologue may be gene comprised any of the following strains.

### Example Pseudomonas Strains

For example, the target cell is a cell of a strain selected from Pseudomonas aeruginosa strain: IOMTU 133, Pseudomonas aeruginosa DSM 50071, Pseudomonas aeruginosa genome assembly NCTC10332, Pseudomonas aeruginosa isolate B10W, Pseudomonas aeruginosa isolate PA14Or, Pseudomonas aeruginosa NCGM2.S1, Pseudomonas aeruginosa PAK, Pseudomonas aeruginosa strain 243931, Pseudomonas aeruginosa strain 24Pae 112, Pseudomonas aeruginosa strain 268, Pseudomonas aeruginosa strain 60503, Pseudomonas aeruginosa strain AR_0095, Pseudomonas aeruginosa strain AR_0353, Pseudomonas aeruginosa strain AR_0354, Pseudomonas aeruginosa strain AR_455, Pseudomonas aeruginosa strain BAMCPA07-48, Pseudomonas aeruginosa strain CCUG 51971, Pseudomonas acruginosa strain E90. Pseudomonas aeruginosa strain FDAARGOS_571. Pseudomonas acruginosa strain GIMC5002:PAT-169. Pseudomonas acruginosa strain H26023. Pseudomonas acruginosa strain L10. Pseudomonas acruginosa strain M1608, Pseudomonas acruginosa strain M37351. Pseudomonas acruginosa strain MRSN12280. Pseudomonas acruginosa strain NCTC13715. Pseudomonas aeruginosa strain Pa58. Pseudomonas aeruginosa strain PABLO48. Pseudomonas acruginosa strain PAK. Pseudomonas acruginosa strain PASGNDM345. Pseudomonas acruginosa strain PASGNDM699. Pseudomonas acruginosa strain PA-VAP-3. Pseudomonas acruginosa strain PB368. Pseudomonas acruginosa strain PB369. Pseudomonas acruginosa strain S04 90. Pseudomonas acruginosa strain ST773.Pseudomonas acruginosa strain T2436, Pseudomonas acruginosa strain W60856. Pseudomonas acruginosa strain WPB099. Pseudomonas aeruginosa strain WPB100. Pseudomonas acruginosa strain WPB101. Pseudomonas acruginosa UCBPP-PA14. Pseudomonas acruginosa UCBPP-PA14. Pseudomonas acruginosa VRFPA04, Pseudomonas amygdali pv. lachrymans str. M301315. Pseudomonas amygdali pv. lachrymans strain NM002. Pseudomonas amygdali pv. morsprunorum strain R15244. Pseudomonas amygdali pv. tabaci str. ATCC 11528. Pseudomonas avellanae strain R2leaf. Pseudomonas coronafaciens pv. coronafaciens strain B19001. Pseudomonas coronafaciens pv. oryzac str. 1_6. Pseudomonas coronafaciens strain X-1. Pseudomonas otitidis MrB4. Pseudomonas salegens strain CECT 8338. Pseudomonas savastanoi pv. phascolicola 1448A. Pseudomonas savastanoi pv. savastanoi NCPPB 3335. Pseudomonas sp. KBS0707. Pseudomonas sp. LPHI. Pseudomonas syringae CC1557. Pseudomonas syringae group genomosp. 3 isolate CFBP6411. Pseudomonas syringae isolate CFBP3840. Pseudomonas syringae pv. actinidiac ICMP 18708. Pseudomonas syringae pv. actinidiac ICMP 18884. Pseudomonas syringae pv. actinidiac ICMP 9853. Pseudomonas syringae pv. actinidiac str. Shaanxi_M228. Pseudomonas syringae pv. actinidiac strain CRAFRU 12.29. Pseudomonas syringae pv. actinidiac strain CRAFRU 14.08. Pseudomonas syringae pv. actinidiac strain MAFF212063. Pseudomonas syringae pv. actinidiac strain NZ-45. Pseudomonas syringae pv. actinidiac strain NZ-47. Pseudomonas syringae pv. actinidiac strain P155. Pseudomonas syringae pv. avii isolate CFBP3846. Pseudomonas syringae pv. cerasicola isolate CFBP6109. Pseudomonas syringae pv. maculicola str. ES4326. Pseudomonas syringae pv. tomato str. DC3000. Pseudomonas syringae pv. tomato strain B13-200. Pseudomonas syringae pv. tomato strain delta IV/IX. Pseudomonas syringae pv. tomato strain delta VI. Pseudomonas syringae pv. tomato strain delta X. Pseudomonas syringae strain CFBP 2116 and Pseudomonas syringae strain Ps25, which strains have NCBI Accession Numbers respectively of AP017302.1. CP012001.1. LN831024.1. CP017969.1. LT608330.1. AP012280.1. CP020659.1. CP041772.1. CP029605.1. CP032761.1. CP041774.1. CP027538.1. CP027172.1. CP027171.1. CP030328.1. CP015377.1. CP043328.1. CP044006.1. CP033833.1. CP043549.1. CP033685.1. CP019338.1. CP008862.2. CP008863.1. CP028162.1. LR134330.1. CP021775.1. CP039293.1. LR657304.1. CP020703.1. CP020704.1. CP028330.1. CP025050.1. CP025049.1. CP011369.1. CP041945.1, CP039988.1, CP008864.2, CP031878.1, CP031877.1, CP031876.1, CP034244.1, CP000438.1, CP008739.2, CP031225.1, CP020351.1, CP026558.1, CP042804.1, CP026562.1, CP046441.1, CP046035.1, CP050260.1, AP022642.1, LT629787.1, CP000058.1, CP008742.1, CP041754.1, CP017290.1, CP007014.1, LT963408.1, LT963409.1, CP012179.1, CP011972.2, CP018202.1, CP032631.1, CP019730.1, CP019732.1, CP024712.1, CP017007.1, CP017009.1, CP032871.1, LT963402.1, LT963391.1, CP047260.1, AE016853.1, CP019871.1, CP047072.1, CP047071.1, CP047073.1, LT985192.1 and CP034558.1, or an orthologue or homologue of such a pump. Optionally, such RND efflux protein is encoded by gene P. syringae PSPTO_0820 or an orthologue or homologue thereof.

### Example Non-Pseudomonas Strains

For example, the target cell is a cell of a strain selected from Azotobacter chroococcum NCIMB 8003, Azotobacter chroococcum strain B3, Azotobacter salinestris strain KACC 13899, Burkholderia ambifaria MC40-6, Burkholderia cenocepacia AU 1054 chromosome 1, Burkholderia cenocepacia HI2424 chromosome 3, Burkholderia cenocepacia MC0-3, Burkholderia cenocepacia strain CR318 chromosome 3, Burkholderia cenocepacia strain FDAARGOS_720, Burkholderia lata strain A05, Burkholderia pyrrocinia strain mHSR5, Cupriavidus basilensis strain 4G11, Cupriavidus necator N-1 plasmid pBB1, Cupriavidus taiwanensis STM 3679, Lysobacter gummosus strain 3.2.11, Paraburkholderia sprentiae WSM5005, Paraburkholderia terricola strain mHS1, Ralstonia pseudosolanacearum strain CRMRs218, Ralstonia solanacearum strain UA-1591, Variovorax paradoxus S110, Variovorax sp. PBL-H6, Xanthomonas arboricola pv. juglandis strain Xaj 417, Xanthomonas arboricola pv. pruni strain 15-088, Xanthomonas arboricola strain 17, Xanthomonas axonopodis pv. dieffenbachiae LMG 695, Xanthomonas axonopodis pv. phaseoli strain ISO18C8, Xanthomonas axonopodis pv. phaseoli strain ISO98C12, Xanthomonas campestris pv. campestris MAFF302021, Xanthomonas citri pv. glycines strain 2098, Xanthomonas cuvesicatoria strain LMG930, Xanthomonas perforans strain LH3 and Xanthomonas sp. ISO98C4, which strains have NCBI Accession Numbers respectively of CP010415.1, CP011835.1, CP045302.1, CP001027.1, CP000378.1, CP000460.1, CP000960.1, CP017240.1, CP050980.1, CP024945.1, CP024903.1, CP010537.1, CP002879.1, LT984803.1, CP011131.1, CP017561.1, CP024941.1, CP021764.1, CP034195.1, CP001636.1, LR594659.1, CP012251.1, CP044334.1, CP011256.1, CP014347.1, CP012063.1, CP012057.1, AP019684.1, CP041965.1, CP018467.1, CP018475.1 and CP012060.1.

For example, the target cell comprises a RND efflux pump or RND efflux pump protein of a strain selected from
(a) Pseudomonas aeruginosa strain: IOMTU 133, Pseudomonas aeruginosa DSM 50071, Pseudomonas aeruginosa genome assembly NCTC10332, Pseudomonas aeruginosa isolate B10W, Pseudomonas acruginosa isolate PA14Or, Pseudomonas acruginosa NCGM2.S1. Pseudomonas acruginosa PAK. Pseudomonas acruginosa strain 243931. Pseudomonas acruginosa strain 24Pac112. Pseudomonas acruginosa strain 268. Pseudomonas acruginosa strain 60503. Pseudomonas acruginosa strain AR_0095. Pseudomonas acruginosa strain AR_0353. Pseudomonas acruginosa strain AR_0354. Pseudomonas acruginosa strain AR_455. Pseudomonas acruginosa strain BAMCPA07-48. Pseudomonas acruginosa strain CCUG 51971. Pseudomonas acruginosa strain E90. Pseudomonas acruginosa strain FDAARGOS_571. Pseudomonas acruginosa strain GIMC5002:PAT-169. Pseudomonas acruginosa strain H26023. Pseudomonas acruginosa strain L10, Pseudomonas acruginosa strain M1608. Pseudomonas acruginosa strain M37351. Pseudomonas acruginosa strain MRSN12280. Pseudomonas acruginosa strain NCTC13715. Pseudomonas acruginosa strain Pa58. Pseudomonas acruginosa strain PABLO48. Pseudomonas acruginosa strain PAK. Pseudomonas acruginosa strain PASGNDM345. Pseudomonas acruginosa strain PASGNDM699. Pseudomonas acruginosa strain PA-VAP-3. Pseudomonas acruginosa strain PB368. Pseudomonas acruginosa strain PB369. Pseudomonas acruginosa strain S04 90. Pseudomonas aeruginosa strain ST773.Pseudomonas acruginosa strain T2436. Pseudomonas acruginosa strain W60856. Pseudomonas acruginosa strain WPB099. Pseudomonas acruginosa strain WPB100. Pseudomonas aeruginosa strain WPB101. Pseudomonas acruginosa UCBPP-PA14. Pseudomonas acruginosa UCBPP-PA 14. Pseudomonas acruginosa VRFPA04. Pseudomonas amygdali pv. lachrymans str. M301315. Pseudomonas amygdali pv. lachrymans strain NM002. Pseudomonas amygdali pv. morsprunorum strain R15244. Pseudomonas amygdali pv. tabaci str. ATCC 11528. Pseudomonas avellanac strain R2leaf. Pseudomonas coronafaciens pv. coronafaciens strain B19001. Pseudomonas coronafaciens pv. oryzac str. 1_6. Pseudomonas coronafaciens strain X-1. Pseudomonas otitidis MrB4. Pseudomonas salegens strain CECT 8338. Pseudomonas savastanoi pv. phascolicola 1448A. Pseudomonas savastanoi pv. savastanoi NCPPB 3335. Pseudomonas sp. KBS0707. Pseudomonas sp. LPHI. Pseudomonas syringae CC1557. Pseudomonas syringae group genomosp. 3 isolate CFBP6411. Pseudomonas syringae isolate CFBP3840. Pseudomonas syringae pv. actinidiac ICMP 18708. Pseudomonas syringae pv. actinidiac ICMP 18884. Pseudomonas syringae pv. actinidiac ICMP 9853. Pseudomonas syringae pv. actinidiac str. Shaanxi_M228. Pseudomonas syringae pv. actinidiac strain CRAFRU 12.29. Pseudomonas syringae pv. actinidiac strain CRAFRU 14.08. Pseudomonas syringae pv. actinidiac strain MAFF212063. Pseudomonas syringae pv. actinidiac strain NZ-45. Pseudomonas syringae pv. actinidiac strain NZ-47. Pseudomonas syringae pv. actinidiae strain P155. Pseudomonas syringae pv. avii isolate CFBP3846. Pseudomonas syringae pv. cerasicola isolate CFBP6109. Pseudomonas syringae pv. maculicola str. ES4326. Pseudomonas syringae pv. tomato str. DC3000. Pseudomonas syringae pv. tomato strain B13-200. Pseudomonas syringae pv. tomato strain delta IV/IX. Pseudomonas syringae pv. tomato strain delta VI. Pseudomonas syringae pv. tomato strain delta X. Pseudomonas syringae strain CFBP 2116 and Pseudomonas syringae strain Ps25, which strains have NCBI Accession Numbers respectively of AP017302.1. CP012001.1. LN831024.1. CP017969.1. LT608330.1. AP012280.1. CP020659.1. CP041772.1. CP029605.1. CP032761.1. CP041774.1. CP027538.1. CP027172.1. CP027171.1. CP030328.1. CP015377.1. CP043328.1. CP044006.1. CP033833.1. CP043549.1. CP033685.1. CP019338.1. CP008862.2. CP008863.1. CP028162.1. LR134330.1. CP021775.1. CP039293.1. LR657304.1. CP020703.1. CP020704.1. CP028330.1. CP025050.1. CP025049.1. CP011369.1. CP041945.1. CP039988.1. CP008864.2. CP031878.1. CP031877.1. CP031876.1. CP034244.1. CP000438.1. CP008739.2. CP031225.1. CP020351.1. CP026558.1. CP042804.1. CP026562.1. CP046441.1. CP046035.1. CP050260.1. AP022642.1. LT629787.1. CP000058.1. CP008742.1. CP041754.1. CP017290.1. CP007014.1. LT963408.1. LT963409.1. CP012179.1. CP011972.2. CP018202.1. CP032631.1. CP019730.1. CP019732.1. CP024712.1. CP017007.1. CP017009.1. CP032871.1. LT963402.1. LT963391.1. CP047260.1. AE016853.1. CP019871.1. CP047072.1. CP047071.1. CP047073.1. LT985192.1 and CP034558.1, or an orthologue or homologue of such a pump: or
(b) Azotobacter chroococcum NCIMB 8003. Azotobacter chroococcum strain B3. Azotobacter salinestris strain KACC 13899. Burkholderia ambifaria MC40-6. Burkholderia cenocepacia AU 1054 chromosome 1. Burkholderia cenocepacia HI2424 chromosome 3. Burkholderia cenocepacia MC0-3. Burkholderia cenocepacia strain CR318 chromosome 3. Burkholderia cenocepacia strain FDAARGOS_720. Burkholderia lata strain A05. Burkholderia pyrrocinia strain mHSR5. Cupriavidus basilensis strain 4G11. Cupriavidus necator N-1 plasmid pBB1. Cupriavidus taiwanensis STM 3679. Lysobacter gummosus strain 3.2.11. Paraburkholderia sprentiae WSM5005. Paraburkholderia terricola strain mHS1. Ralstonia pseudosolanacearum strain CRMRs218. Ralstonia solanacearum strain UA-1591. Variovorax paradoxus S110. Variovorax sp. PBL-H6. Xanthomonas arboricola pv. juglandis strain Xaj 417. Xanthomonas arboricola pv. pruni strain 15-088. Xanthomonas arboricola strain 17. Xanthomonas axonopodis pv. dieffenbachiae LMG 695. Xanthomonas axonopodis pv. phascoli strain ISO18C8. Xanthomonas axonopodis pv. phascoli strain ISO98C12. Xanthomonas campestris pv. campestris MAFF302021. Xanthomonas citri pv. glycines strain 2098, Xanthomonas cuvesicatoria strain LMG930. Xanthomonas perforans strain LH3 and Xanthomonas sp. ISO98C4, which strains have NCBI Accession Numbers respectively of CP010415.1. CP011835.1. CP045302.1. CP001027.1. CP000378.1. CP000460.1. CP000960.1. CP017240.1. CP050980.1. CP024945.1. CP024903.1. CP010537.1. CP002879.1. LT984803.1. CP011131.1. CP017561.1. CP024941.1. CP021764.1. CP034195.1. CP001636.1. LR594659.1. CP012251.1. CP044334.1. CP011256.1. CP014347.1. CP012063.1. CP012057.1. AP019684.1. CP041965.1. CP018467.1. CP018475.1 and CP012060.1, or an orthologue or homologue of such a pump. Optionally, such RND efflux protein is encoded by gene P. syringae PSPTO_0820 or an orthologue or homologue thereof.

For example, the target cell comprises an RND efflux pump protein encoded by gene P. syringae PSPTO_0820 or an orthologue or homologue thereof.

For example, when the target cell comprises an RND efflux pump protein encoded by gene P. syringae PSPTO_0820 or an orthologue or homologue thereof: The target cell is an Azotobacter, Burkholderia, Cupriavidus, Lysobacter, Paraburkholderia, Ralstonia, Variovorax, Xanthomonas or Pseudomonas cell. For example, the target cell is a cell of a Pseudomonas species, optionally wherein the species is selected from Pseudomonas aeruginosa Pseudomonas amygdali, Pseudomonas asturiensis, Pseudomonas avellanae, Pseudomonas cerasi, Pseudomonas chlororaphis, Pseudomonas cichorii, Pseudomonas coronafaciens, Pseudomonas otitidis, Pseudomonas putida, Pseudomonas salegens Pseudomonas savastanoi, Pseudomonas syringae and Pseudomonas viridiflava. For example, the target cell is a cell of a species selected from Azotobacter chroococcum, Azotobacter salinestris, Burkholderia ambifaria, Burkholderia cenocepacia, Burkholderia lata, Burkholderia pyrrocinia, Cupriavidus basilensis, Cupriavidus necator, Cupriavidus taiwanensis, Lysobacter gummosus, Paraburkholderia sprentiae, Paraburkholderia terricola, Ralstonia pseudosolanacearum, Ralstonia solanacearum, Variovorax paradoxus, Xanthomonas arboricola, Xanthomonas axonopodis, Xanthomonas campestris Xanthomonas citri, Xanthomonas euvesicatoria and Xanthomonas perforans.

### PSPTO_4977, Orthologues & Homologues

A PSPTO_4977 gene orthologue or homologue may be gene comprised any of the following strains.

### Example Non-Pseudomonas Strains

For example, the target cell is a cell of a strain selected from Stenotrophomonas rhizophila strain GA1, Enterococcus faecalis strain V583 and Paucimonas lemoignei strain NCTC10937, which strains have NCBI Accession Numbers respectively CP031729.1, CP022312.1 and LS483371.1.

### Example Pseudomonas Strains

For example, the target cell is a cell of a strain selected from Pseudomonas amygdali pv. lachrymans strain NM002, Pseudomonas amygdali pv. morsprunorum strain R15244, Pseudomonas amygdali pv. tabaci str. ATCC 11528, Pseudomonas asturiensis strain CC1524, Pseudomonas avellanae strain R2, Pseudomonas cerasi isolate PL963, Pseudomonas chlororaphis strain PCL1606, Pseudomonas chlororaphis subsp. aurantiaca strain JD37, Pseudomonas chlororaphis subsp. aureofaciens strain ChPhzTR36, Pseudomonas chlororaphis subsp. chlororaphis strain DSM 50083, Pseudomonas chlororaphis subsp. piscium strain DSM 21509, Pseudomonas cichorii JBC1, Pseudomonas coronafaciens pv. coronafaciens strain B19001, Pseudomonas putida GB-1 chromosome, Pseudomonas savastanoi pv. phaseolicola 1448A, Pseudomonas sp. 09C 129, Pseudomonas syringae CC1557, Pseudomonas syringae pv. actinidiae ICMP 18708, Pseudomonas syringae pv. cerasicola isolate CFBP6109, Pseudomonas syringae pv. lapsa strain ATCC 10859, Pseudomonas syringae pv. maculicola str. ES4326, Pseudomonas syringae pv. pisi str. PP1, Pseudomonas syringae pv. syringae B301D, Pseudomonas syringae pv. syringae B301D, Pseudomonas syringae UMAF0158 and Pseudomonas viridiflava strain CFBP 1590, which strains have NCBI Accession Numbers respectively of CP020351.1, CP026558.1, CP042804.1, CP047265.1, CP026562.1, LT963395.1. CP011110.1, CP009290.1, CP027721.1, CP027712.1, CP027707.1, CP007039.1, CP046441.1, CP000926.1, CP000058.1, CP025261.1, CP007014.1, CP012179.1, LT963391.1, CP013183.1, CP047260.1, CP034078.1, CP005969.1, AE016853.1, CP005970.1 and LT855380.1.

For example, the target cell comprises a RND efflux pump or RND efflux pump protein of a strain selected from
(a) Stenotrophomonas rhizophila strain GA1, Enterococcus faecalis strain V583 and Paucimonas lemoignei strain NCTC10937, which strains have NCBI Accession Numbers respectively CP031729.1, CP022312.1 and LS483371.1, or an orthologue or homologue of such a pump: or
(b) Pseudomonas amygdali pv. lachrymans strain NM002, Pseudomonas amygdali pv. morsprunorum strain R15244, Pseudomonas amygdali pv. tabaci str. ATCC 11528, Pseudomonas asturiensis strain CC1524, Pseudomonas avellanae strain R2, Pseudomonas cerasi isolate PL963, Pseudomonas chlororaphis strain PCL1606, Pseudomonas chlororaphis subsp. aurantiaca strain JD37, Pseudomonas chlororaphis subsp. aurcofaciens strain ChPhzTR36, Pseudomonas chlororaphis subsp. chlororaphis strain DSM 50083, Pseudomonas chlororaphis subsp. piscium strain DSM 21509, Pseudomonas cichorii JBC1, Pseudomonas coronafaciens pv. coronafaciens strain B19001, Pseudomonas putida GB-1 chromosome, Pseudomonas savastanoi pv. phaseolicola 1448A, Pseudomonas sp. 09C 129, Pseudomonas syringae CC1557, Pseudomonas syringae pv. actinidiae ICMP 18708, Pseudomonas syringae pv. cerasicola isolate CFBP6109, Pseudomonas syringae pv. lapsa strain ATCC 10859, Pseudomonas syringae pv. maculicola str. ES4326, Pseudomonas syringae pv. pisi str. PP1, Pseudomonas syringae pv. syringae B301D, Pseudomonas syringae pv. syringae B301D, Pseudomonas syringae UMAF0158 and Pseudomonas viridiflava strain CFBP 1590, which strains have NCBI Accession Numbers respectively of CP020351.1, CP026558.1, CP042804.1. CP047265.1, CP026562.1, LT963395.1, CP011110.1, CP009290.1, CP027721.1, CP027712.1. CP027707.1, CP007039.1, CP046441.1, CP000926.1, CP000058.1, CP025261.1, CP007014.1, CP012179.1, LT963391.1, CP013183.1, CP047260.1, CP034078.1, CP005969.1, AE016853.1. CP005970.1 and LT855380.1, or an orthologue or homologue of such a pump.

For example, the target cell comprises an RND efflux pump protein encoded by gene P. syringae PSPTO_4977 or an orthologue or homologue thereof.

For example, when the target cell comprises an RND efflux pump protein encoded by gene P. syringae PSPTO_4977 or an orthologue or homologue thereof: The target cell is a Stenotrophomonas, Enterococcus, Paucimonas or Pseudomonas cell. For example, the target cell is a cell of a Pseudomonas species, optionally wherein the species is selected from Pseudomonas amygdali, Pseudomonas asturiensis, Pseudomonas avellanae, Pseudomonas cerasi, Pseudomonas chlororaphis, Pseudomonas cichorii, Pseudomonas coronafaciens, Pseudomonas putida, Pseudomonas savastanoi, Pseudomonas syringae and Pseudomonas viridiflava. For example, the target cell is a cell of a species selected from Stenotrophomonas rhizophila, Enterococcus faecalis, Paucimonas lemoignei, Pseudomonas amygdali, Pseudomonas asturiensis, Pseudomonas avellanae, Pseudomonas cerasi, Pseudomonas chlororaphis, Pseudomonas cichorii, Pseudomonas coronafaciens, Pseudomonas putida, Pseudomonas savastanoi, Pseudomonas syringae and Pseudomonas viridiflava.

### Example Carrier Cells

For example, each carrier cell is a gram-positive bacterial cell. For example, each carrier cell is a gram-negative bacterial cell. For example, the carrier cell is a cell of a genus or species disclosed in Table 1 of WO2017211753 (the disclosure of this table and each genus and species individually being incorporated herein for disclosure of cell genus or species that may be used in the present invention).

For example, the carrier cell is a cell of phylum Proteobacteria, class Gammaproteobacteria, order Pseudomonadales or family Pseudomonadaceae. In a preferred example, the carrier is a Pseudomonas (eg, P. fluorscens) cell.

For example, the carrier is an E. coli cell (eg, E. coli, K12, Nissle or S17 cell,).

For example, the carrier is a gram positive cell, eg, a Bacillus (such as Bacillus subtilis) or Cloistridiales (such as Clostridium butyricum) cell.

In an example, the subject is a shellfish. The shellfish may be selected from shrimp, crayfish, crab, lobster, clam, scallop, oyster, prawn and mussel.

The subject may be any subject disclosed herein. The subject may be an animal, such as a livestock animal, eg, a bird (such as a poultry bird: or a chicken or a turkey) or swine,

In an alternative, the subject is a plant, eg, and the target bacteria are plant pathogen bacteria. In an example, the target baceteria are Pseudomonas, eg, P. syringae or P. aeruginosa.

In an alternative, the carrier and target cells are archaeal cells. For example the target cells are methanobacterium cells. For example the target cells are methanogen cells. For example, the target cells comprise one or more species of cell selected from:
Methanobacterium bryantii
Methanobacterium formicum
Methanobrevibacter arboriphilicus
Methanobrevibacter gottschalkii
Methanobrevibacter ruminantium
Methanobrevibacter smithii
Methanococcus chunghsingensis
Methanococcus burtonii
Methanococcus aeolicus
Methanococcus deltae
Methanococcus jannaschii
Methanococcus maripaludis
Methanococcus vannielii
Methanocorpusculum labreanum
Methanoculleus bourgensis (Methanogenium olentangyi & Methanogenium bourgense)
Methanoculleus marisnigri
Methanoflorens stordalenmirensis[34]
Methanofollis liminatans
Methanogenium cariaci
Methanogenium frigidum
Methanogenium organophilum
Methanogenium wolfei
Methanomicrobium mobile
Methanopyrus kandleri
Methanoregula boonei
Methanosaeta concilii
Methanosaeta thermophila
Methanosarcina acetivorans
Methanosarcina barkeri
Methanosarcina mazei
Methanosphaera stadtmanae
Methanospirillium hungatei
Methanothermobacter defluvii (Methanobacterium defluvii)
Methanothermobacter thermautotrophicus (Methanobacterium thermoautotrophicum)
Methanothermobacter thermoflexus (Methanobacterium thermoflexum)
Methanothermobacter wolfei (Methanobacterium wolfei)
Methanothrix sochngenii

Optionally, the target cells are not pathogenic to the subject, for example when the method is a non-medical method. In an example, the method is a cosmetic method.

For example, the target cells are methane-producing cells, and optionally the subject is a livestock animal, preferably a ruminant, or a cow (eg, a beef or dairy cattle). By reducing methane-producing cells in such animal, the invention may in one embodiment enhance the weight of the animal (eg, enhance the yield of meat from the animal) and/or enhance the yield of milk or another product of the animal, such as fur or fat.

In an example, the target cells are selected from E. coli, Salmonella and Campylobacter cells. In an example, the target cells are E. coli, Salmonella or Campylobacter cells. In an example, each animal is a chicken (eg, a broiler or hen-layer) and the target cells are Salmonella or Campylobacter cells. In an example, each animal is a cow (eg, a beef or dairy cow) and the target cells are methanogen cells.

In an example, the target cells are selected from Mycoplasma (eg, Mycoplasma mycoides (eg, Mycoplasma mycoides subsp. Mycoides), Mycoplasma leachii or Mycoplasma bovis), Brucella abortus, Listeria monocytogenes, Clostridium (eg, Clostridium chauvoei or Clostridium septicum), Leptospira (eg, L. canicola, L. icterohaemorrhagiae, L. grippotyphosa, L. hardjo or L. Pomona), Mannheimia haemolytica, Trueperella pyogenes, Mycobacterium bovis, Campylobacter spp. (eg, Campylobacter jejuni or Campylobacter coli), Bacillus anthracis, E. coli (eg, E. coli O157:H7) or Pasteurella multocida (eg, Pasteurella multocida B:2, E:2, A: 1 or A:3). In the example, optionally the subject or animal is a livestock animal, such as a cow, sheep, goat or chicken (preferably a cow).

Optionally, eg, wherein the subject is an animal (eg, a livestock animal or a wild animal), the target cells are zoonotic bacterial cells, such as cells of a species selected from Bacillus anthracis, Mycobacterium bovis (eg, wherein the animal is a cow), Campylobacter spp (eg. wherein the animal is a poultry animal), Mycobacterium marinum (eg. wherein the animal is a fish), Shiga toxin-producing E. coli (eg. wherein the animal is a ruminant), Listeria spp (eg, wherein the animal is a cow or sheep), Chlamydia abortus (eg, wherein the animal is a sheep), Coxiella burnetii (eg, wherein the animal is a cow, sheep or goat), Salmonella spp (eg, wherein the animal is a poultry animal), Streptococcus suis (eg, wherein the animal is a pig) and Corynebacterium (eg, C. ulcerans) (eg, wherein the animal is a cow).

In an example, a plurality of carrier cells as described herein (eg, carrier cells of any configuration, aspect, example or embodiment described herein) is administered to the subject, wherein the carrier cells comprise the plasmid DNA encoding the agent.

In an example, each animal is a chicken (eg, a broiler or hen-layer) and the target cells are Salmonella or Campylobacter cells. In an example, each animal is a cow (eg, a beef or dairy cow) and the target cells are methanogen cells.

Optionally, the target cells are Salmonella cells. In an example, the target cells comprise S. enterica and/or S. typhimurium cells: optionally wherein the S. enterica is S. enterica subspecies enterica. Optionally, the method kills a plurality of different S. enterica subspecies enterica serovars: optionally wherein each serovar is selected from the group consisting of Typhimurium, Enteritidis, Virchow, Montevideo, Heidelberg, Hadar, Binza, Bredeney, Infantis, Kentucky, Seftenberg, Mbandaka, Anatum, Agona and Dublin. Optionally, the method kills S. enterica subspecies enterica serovars Typhimurium, Infantis and Enteritidis. Optionally, the method kills S. enterica subspecies enterica serovars Typhimurium and Enteritidis. Optionally, the method kills S. enterica subspecies enterica serovars Typhimurium and Infantis. Optionally, the method kills S. enterica subspecies enterica serovars Enteritidis and Infantis. The most prevalent serovars in chicken are Salmonella Enteritidis, Salmonella Infantis and Salmonella Typhimurium. In general, similar serovars of Salmonella are found in infected humans and chicken (S. Enteritidis and S. Typhimurium). By killing Salmonella in livestock animals, the invention is useful for reducing the pool of zoonotic bacteria that are available for transmission to humans (such as by eating the livestock or products made thereofrom, such as meat or dairy products for human consumption).

Advantageously, the carrier cells are Enterobacteriaceae cells, optionally E. coli cells. Optionally, the method kills S. enterica subspecies enterica serovars Typhimurium and Enteritidis serovars.

Optionally the method reduces target cells in the gastrointestinal tract of the animal: optionally the method reduces target cells in the jejunum, ileum, colon, liver, spleen or caecum of the animal; optionally wherein the animal is a bird and the method reduces target cells in the caecum of the bird. This may be important to reduce spread of zoonotic or other deterimental target strains in the faeces of the subjects, such as livestock animals. Thus, in an example the method is carried out on a group of subjects (eg, a herd or flock, such as a herd of swine or a flock of birds), wherein spread of cells of the target species is reduced in the group.

Thus, in an example the method is carried out on a group (optionally a flock or herd) of animals, wherein some or all of the animals comprise target cells (eg, Salmonella cells), wherein spread of cells of the target species is reduced in the group: or wherein spread is reduced from the group to a second group of animals.

Optionally, the plasmid comprises a RP4 origin of transfer (oriT). The plasmid may be any type of plasmid disclosed herein.

The agent may be any antibacterial agent disclosed herein, preferably a guided nuclease that is programmed to cut one or more target sequences in target cells. A suitable nuclease may be a TALEN, meganuclease, zinc finger nuclease or Cas nuclease. For example, the agent comprises one or more components (eg, a Cas nuclease and/or a guide RNA or a crRNA) of a CRISPR/Cas system that is operable in a target cell to cut a protospacer sequence comprised by the target cell, optionally wherein the target cells comprise first and second strains of a bacterial species and each strain comprises the protospacer sequence, wherein cells of the strains are killed. For example, the system is operable to cut at least 3 different protospacer sequences comprised by the cell genome. Optionally, each or some of said protospacer sequences is comprised by a pathogenicity island that is comprised by the cell. Optionally, the agent is operable to cut a plurality of different protospacer sequences comprised by the target cell genome. Optionally, the agent comprises one or more components of a CRISPR/Cas system that is operable in a target cell to cut at least 2, 3, 4, 5, 6, 7, 8, 9, or 10 different protospacer sequences comprised by the target cell genome (eg, comprised by the target cell chromosome).

In an embodiment, the agent
(a) comprises a guided nuclease that is capable of recognising and modifying a target nucleic acid sequence, wherein the target sequence is comprised by an endogenous chromosome or episome of the target cells but is not comprised by the carrier cells, wherein the nuclease modifies the chromosome or episome to kill the target cells or inhibit the growth or proliferation of the target cells; and/or
(b) encodes a guide RNA or crRNA of a CRISPR/Cas system that operates with a Cas nuclease in the target cells to cut a protospacer sequence comprised by the target cells.

Optionally, each target cell is a Salmonella cell and each carrier cell is an Enterobacteriaceae cell.

Optionally, the target cell are cells of a species or strain that is pathogenic to the subject and the method treats or reduces a symptom of an infection by pathogenic target cells.

Any administration of cells to a subject herein may be by oral administration. Any administration of cells to a subject herein may preferably be by administration to the GI tract. Any administration of cells to a subject herein may be by systemic, intranasal or inhaled administration.

### There is Also Provided:

A non-medical method of killing zoonotic bacterial target cells in an animal, the method comprising administering to the animal a plurality of the carrier cells, wherein said plasmids are transferred from carrier cells into target cells for expression therein to produce the antibacterial agent, thereby killing target cells in the subject or reducing the growth or proliferation of target cells, optionally wherein the target cells are Salmonella cells and/or the carrier cells are Enterobacteriaceae cells.

The animal may be any animal disclosed herein, eg, a livestock animal, domesticated animal or wild animal (eg, a bat or bird)).

Optionally, any method herein reduces Salmonella in the gastrointestinal tract of the subject.

Optionally, the target cells comprise different Salmonella spp. types that are killed.

There is provided the following definitions:-

Homologue: A gene, nucleotide or protein sequence related to a second gene, nucleotide or protein sequence by descent from a common ancestral DNA or protein sequence. The term, homologue, may apply to the relationship between genes separated by the event of or to the relationship between genes separated by the event of genetic duplication.

Orthologue: Orthologues are genes, nucleotide or protein sequences in different species that evolved from a common ancestral gene, nucleotide or protein sequence by speciation. Normally, orthologues retain the same function in the course of evolution.

Optionally any Salmonella herein is Salmonella enterica subsp. enterica serovar Typhimurium str. LT2.

Optionally, each plasmid encodes a plurality of guide RNAs or crRNAs of a CRISPR/Cas system wherein the guide RNAs or crRNAs are operable with Cas nuclease in the target cell to recognise a plurality of protospacer sequences comprised by the target cell genome, optionally wherein the target cell is a Salmonella cell and the protospacer sequences comprise one or more nucleotide sequences of genes selected from invB, sicP and sseE. For example, the protospacer sequences comprise nucleotide sequences of genes invB, sicP and sseE. In an example, the plasmid also encodes a Cas, eg, a Cas9, Cas3, Cpf1, Cas12, Cas13, CasX or CasY. In an embodiment, the Cas is a Type I, II, III, IV, V or VI Cas, preferably a Type I or II Cas. In an example, the DNA also encodes a Cas3 and cognate Cascade proteins (eg, CasA, B, C, D and E). Optionally, the Cas (and Cascade of present) are E. coli Cas (and Cascade).

The plasmid may comprise one or more CRISPR spacers, wherein each spacer consists of 20-40, 25-35, or 30-35 consecutive nucleotides of a gene comprised by the genome of the target cell: eg,
(a) a gene selected from avrA, sptP, sicP, sipA, sipD, sipC, sipB, sicA, invB, ssaE, sseA, sseB, sscA, sseC, sseD, sseE, sscB, sseF, sseG, mgtC, cigR, pipA, pipB, pipC, sopB and pipD of Salmonella or a homologue or orthologue thereof;
(b) a gene comprised by a pathogenicity island that is comprised by the target cell genome;
(c) a secretion system (eg, a type III protein secretion system) gene comprised by the target cell genome.

Optionally, the plasmid comprises a RP4 origin of transfer (oriT) and/or a p15A origin of replication.

In an example, the plasmid is a conjugative phagemid.

In an example, the plasmid encodes a Cas3 and optionally one or more Cascade proteins (eg, one or more of CasA, B, C, D and E). In an embodiment, the plasmid encodes a Cas3 and CasA, B, C, D and E. In an embodiment, the plasmid encodes an E. coli Cas3 and CasA, B, C, D and E. Optionally, the guided nuclease (eg, Cas3) is a Type I-A, -B, -C, -D, -E, -F or -U Cas.

In an example, the agent in any configuration, aspect, example, option or embodiment herein, the agent comprises one or more components of a CRISPR/Cas system that is operable in the target cell to cut a protospacer sequence comprised by the target cell.

In an example, the system is operable to cut at least 3 different protospacer sequences comprised by the target cell genome. In an embodiment, each or some of said protospacer sequences is comprised by a pathogenicity island that is comprised by the target cell.

In an example, the plasmid
(a) encodes a guided nuclease that is capable of recognising and modifying a target cell nucleic acid sequence, wherein the target sequence is comprised by an endogenous chromosome or episome of the target cell(s) but is not comprised by the carrier cell(s), wherein the nuclease modifies the chromosome or episome to kill the target cell(s) or inhibit the growth or proliferation of the target cell(s); and/or
(b) encodes a guide RNA or crRNA of a CRISPR/Cas system that operates with a Cas nuclease in the target cell to cut a protospacer sequence comprised by the target cell.

Optionally, the plasmid comprises a constitutive promoter for expression of the guide RNAs or crRNAs. Optionally, the plasmid comprises a constitutive promoter for expression of a Cas nuclease that is operable in a target cell with the guide RNAs or crRNAs to modify (eg, cut) protospacer sequences of the target cell genome.

Optionally, the Cas, Cascade proteins, gRNAs and crRNAs are E. coli K12 (MG1655) Cas, Cascade proteins, gRNAs and crRNAs respectively. Optionally, the plasmid is devoid of nucleotide sequences encoding Cas1 and Cas2 proteins.

In embodiments, the growth or proliferation of target cells is reduced (eg, by at least 40, 50, 60, 70, 80, or 90% compared to growth in the absence of the agent). The invention finds application, for example, in controlling or killing target bacteria that are pathogenic to humans, animals or plants. The invention finds application, for example, in controlling or killing zoonotic target bacteria comprised by an animal (eg, a livestock animal). For example, the carrier cells may be comprised by a medicament for treating or preventing a disease or condition in a human or animal: a growth promoting agent for administration to animals for promoting growth thereof: killing zoonositic bacteria in the animals: for administration to livestock as a pesticide: a pesticide to be applied to plants: or a plant fertilizer.

An advantage may be that the carrier cells may be used as producer cells in which DNA encoding the antibacterial agent can be replicated.

### Example Plasmids

A method of delivery of any agent, such as a CRISPR-Cas system (or a component thereof) can be by bacterial conjugation, a natural process whereby a donor bacterium (carrier bacterium) transfers plasmid DNA from itself to a recipient bacterium (target bacterium). Donor bacteria elaborate a surface structure, the pilus which can be considered to be like a syringe or drinking straw through which the DNA is delivered. The donor pilus binds to the surface of a receptive recipient and this event triggers the process of DNA transfer. Plasmids are suitable for this conjugative process, where the plasmid comprises DNA enoding the agent of the invention.

DNA transfer by conjugation may only take place with a ‘susceptible recipient’ but does not generally occur with a recipient carrying a similar type of plasmid. Because conjugation is via pilus bridge, it is possible for that bridge to attach itself not to a recipient but to the donor bacterium. This could result in a futile cycle of transfer of the plasmid DNA to itself. Plasmids thus naturally encode incompatibility factors. One is a surface arrayed protein that prevents the pilus binding to bacterium displaying that surface protein such as itself or any other bacterium carrying the same plasmid. Additionally, plasmids naturally encode another incompatibility system that closely regulates the copy number of the plasmid inside a bacterium. Thus, should a conjugation event manage to evade surface exclusion and start to transfer DNA by conjugation, the recipient will prevent that plasmid establishing as it already maintains the current copy number and will not accept and maintain a further unwanted additional copy.

In an example of the invention, the plasmid is a member of a plasmid incompatibility group, wherein the target cell does not comprise a plasmid of said group. Optionally, the plasmid of the invention is a member of the incompatibility group P (ie, the plasmid is an incP plasmid). Salmonella very rarely carry incP plasmids, so this incP plasmid is useful where the target cell is a Salmonella cell. For example within the Enterobacteriaceae the following is a non-exclusive list of potential plasmids that could use for delivery: IncFI, IncFII, IncFIll, IncFIV, IncFV, IncM, Inc9, InclO, Incl, IncA, IncB, IncC, IncH, Incla, Inelle, Ine12, Incly, IncJ, IncL, IncN, Inc2e, IncO, IncP, IncS, IncT and/or IncW. Thus, optionally, the target cell is an Enterobacteriaceae cell and the DNA of the invention is comprised by a plasmid, wherein the plasmid is selected from an IncFI, IncFII, IncFIII, IncFIV, IncFV, IncM, Inc9, InclO, Inel, IncA, IneB, IncC, IncH, Incla, Inelle, Inc12, Incly, IncJ, IncL, IncN, Inc2e, IncO, IncP, IncS, IncT and IncW plasmid.

In an example, the carrier cell of the invention comprises two or more plasmids, each plasmid comprising a DNA that encodes an antibacterial agent, wherein a first of said plasmids is a member of a first incompatibility group, wherein the target cell does not comprise a plasmid of said first group, and wherein a second of said plasmids is a member of a second incompatibility group, wherein the target cell does not comprise a plasmid of said second group. For example, a carrier cell may comprise an incP plasmid encoding an anti-target cell CRISPR-Cas system or a component thereof (eg, encoding a first crRNA or guide RNA that targets a first protospacer sequence of the target cell genome) and wherein the carrier cell further comprises an incF1 plasmid encoding an anti-target cell CRISPR-Cas system or a component thereof (eg, encoding a second crRNA or guide RNA that targets a second protospacer sequence of the target cell genome), the protospacers comprising different nucleotide sequences. For example, the protospacers are comprised by different genes of the target cell genome. For example, the protospacers are comprised by one or more pathogenicity islands of the target cell genome. Optionally, the target cell is an Enterobacteriaceae cell. Optionally, the carrier cell comprises a group of plasmids comprising 2, 3, 4, 5, 6 or more different types of plasmid, wherein each plasmid is capable of being conjugatively transferred into a target cell, wherein the plasmids encode different agents or different components of an antibacterial agent. For example, the plasmids encode different cRNAs or gRNAs that target different protospacers comprisesd by the target cell genome. For example, the group of plasmids comprises up to n different types of plasmid, wherein the plasmids are members of up to n different incompatibility groups, eg. groups selected from IncFI, IncFII, IncFIII, IncFIV, IncFV, IncM, Inc9, InclO, Incl, IncA, IncB, IncC, IncH, IncIa, Incllc, IncI2, Incly, IncJ, IncL, IncN, Inc2e, IncO, IncP, IncS, IncT and IneW. For example, n=2, 3, 4, 5, 6, 7, 8, 9 or 10.

For example, the carrier cell comprises (i) a first plasmid that encodes a first type of CRISPR/Cas system that targets a first protospacer comprised by the target cell genome, or encodes a component of said system; and (ii) a second plasmid that encodes a second type of CRISPR/Cas system that targets a second protospacer comprised by the target cell genome, or encodes a component of said system. wherein the first and second types are different. For example, the first type is a Type I system, and the second type is a Type II system (eg. the first plasmid encodes a Cas3, Cascade and a crRNA or guide RNA that is operable with the Cas3 and Cascade in the target cell to modify the first protospacer; and the second plasmid encodes a Cas9 and a crRNA or guide RNA that is operable with the Cas9 in the target cell to modify the second protospacer). In an alternative, the Cas3 and Cascade are encoded by an endogenous target cell gene, wherein the first plasmid encodes the crRNA or guide RNA that is operable with the endogenous Cas3 and Cascade in the target cell to modify the first protospacer. In an alternative, the Cas9 is encoded by an endogenous target cell gene, wherein the second plasmid encodes the crRNA or guide RNA that is operable with the endogenous Cas9 in the target cell to modify the second protospacer. Optionally, the Cas3 and Cascade are encoded by endogenous genes of the target cell and the Cas9 is encoded by the second plasmid.

Instead of a Type I and Type II system, the invention alternatively provides in an embodiment a first plasmid enocoding a Type I CRISPR/Cas system (or component thereof, eg, a Cas3 or a crRNA or a gRNA) and a second plasmid encoding a Type III CRISPR/Cas system (or a component thereof). Instead of a Type I and Type II system, the invention alternatively provides in an embodiment a first plasmid enocoding a Type I CRISPR/Cas system (or component thereof) and a second plasmid encoding a Type IV CRISPR/Cas system (or a component thereof). Instead of a Type I and Type II system, the invention alternatively provides in an embodiment a first plasmid enocoding a Type I CRISPR/Cas system (or component thereof) and a second plasmid encoding a Type V CRISPR/Cas system (or a component thereof). Instead of a Type I and Type II system, the invention alternatively provides in an embodiment a first plasmid enocoding a Type I CRISPR/Cas system (or component thereof) and a second plasmid encoding a Type VI CRISPR/Cas system (or a component thereof).

Instead of a Type I and Type II system, the invention alternatively provides in an embodiment a first plasmid enocoding a Type II CRISPR/Cas system (or component thereof, eg, a Cas9 or a crRNA or a gRNA) and a second plasmid encoding a Type III CRISPR/Cas system (or a component thereof). Instead of a Type I and Type II system, the invention alternatively provides in an embodiment a first plasmid enocoding a Type II CRISPR/Cas system (or component thereof) and a second plasmid encoding a Type IV CRISPR/Cas system (or a component thereof). Instead of a Type I and Type II system, the invention alternatively provides in an embodiment a first plasmid enocoding a Type II CRISPR/Cas system (or component thereof) and a second plasmid encoding a Type V CRISPR/Cas system (or a component thereof). Instead of a Type I and Type II system, the invention alternatively provides in an embodiment a first plasmid enocoding a Type II CRISPR/Cas system (or component thereof) and a second plasmid encoding a Type VI CRISPR/Cas system (or a component thereof).

Instead of a Type I and Type II system, the invention alternatively provides in an embodiment a first plasmid enocoding a Type V CRISPR/Cas system (or component thereof, eg, a Cas12a or a crRNA) and a second plasmid encoding a Type III CRISPR/Cas system (or a component thereof). Instead of a Type I and Type II system, the invention alternatively provides in an embodiment a first plasmid enocoding a Type V CRISPR/Cas system (or component thereof) and a second plasmid encoding a Type IV CRISPR/Cas system (or a component thereof). Instead of a Type I and Type II system, the invention alternatively provides in an embodiment a first plasmid enocoding a Type V CRISPR/Cas system (or component thereof) and a second plasmid encoding a Type V CRISPR/Cas system (or a component thereof). Instead of a Type I and Type II system, the invention alternatively provides in an embodiment a first plasmid enocoding a Type V CRISPR/Cas system (or component thereof) and a second plasmid encoding a Type VI CRISPR/Cas system (or a component thereof).

Instead of a Type I and Type II system, the invention alternatively provides in an embodiment first and second plasmids, each enocoding a Type I CRISPR/Cas system (or a component thereof). Instead of a Type I and Type II system, the invention alternatively provides in an embodiment first and second plasmids, each cnocoding a Type II CRISPR/Cas system (or a component thereof). Instead of a Type I and Type II system, the invention alternatively provides in an embodiment first and second plasmids, each enocoding a Type III CRISPR/Cas system (or a component thereof). Instead of a Type I and Type II system, the invention alternatively provides in an embodiment first and second plasmids, each enocoding a Type IV CRISPR/Cas system (or a component thereof). Instead of a Type I and Type II system, the invention alternatively provides in an embodiment first and second plasmids, each enocoding a Type V CRISPR/Cas system (or a component thereof). Instead of a Type I and Type II system, the invention alternatively provides in an embodiment first and second plasmids, each enocoding a Type VI CRISPR/Cas system (or a component thereof).

Optionally, the plasmids are members of different incompatibility groups, eg, groups selected from IncFI, IncFII, IncFIII, IncFIV, IncFV, IncM, Inc9, InclO, Incl, IncA, IneB, IncC, IncH, IncIa, Incllc, IncI2, Incly, IncJ, IncL, IncN, Inc2e, IncO, IncP, IncS, IncT and IneW. In an example here, the target cell is an Enterobacteriaceae cell.

Advantageously, the carrier cells are for treating or preventing a target cell infection in a human or an animal subject (eg, a chicken, cow, pig, fish or shellfish). Advantageously, the carrier cells are of a species that is probiotic to said subject or is probioitic to humans or animals (eg, chickens). For example, the carrier cells are probiotic E. coli cell. For example, the carrier cells are probiotic Bacillus cell. In an example, the carrier cells are of a species that is pathogenic to said subject, or is pathogenic to humans or animals (eg, chickens). Advantageously, each plasmid encodes one or more guide RNAs or one or more crRNAs that are capable of hybridizing in the target cell to respective target nucleic acid sequence(s), wherein the target sequence(s) are comprised by an endogenous chromosome and/or endogenous episome of the target cell. For example, each plasmid encodes 2, 3, 4, 5, 6, 7, 7, 9, or 10 (or more than 10) different gRNAs or different crRNAs that hybridise to a respective target sequence, wherein the target sequences are different from each other. For example, 3 different gRNAs or crRNAs are encoded by each plasmid. For example, 2 different gRNAs or crRNAs are encoded by each plasmid. For example, 3 different gRNAs or crRNAs are encoded by each plasmid. For example, 4 different gRNAs or crRNAs are encoded by each plasmid. For example, 3 different gRNAs or crRNAs are encoded by each plasmid. For example, 5 different gRNAs or crRNAs are encoded by each plasmid. For example, 6 different gRNAs or crRNAs are encoded by each plasmid. For example, 7 different gRNAs or crRNAs are encoded by each plasmid. For example, 8 different gRNAs or crRNAs are encoded by each plasmid. For example, 9 different gRNAs or crRNAs are encoded by each plasmid. For example, 10 different gRNAs or crRNAs are encoded by each plasmid. For example, 11 different gRNAs or crRNAs are encoded by each plasmid. For example, 12 different gRNAs or crRNAs are encoded by each plasmid. For example, 13 different gRNAs or crRNAs are encoded by each plasmid. In an example, the target cells are Salmonella cells (eg, wherein the subject is a chicken). In an example, the target cells are E. coli cells. In an example, the target cells are Campylobacter cells (eg, wherein the subject is a chicken). In an example, the target cells are Edwardsiella cells (eg, wherein the subject is a fish or shellfish, eg, a catfish or a shrimp or prawn). In an example, the target cells are E. coli cells.

Optionally, each plasimid comprises an expressible tra1 and/or tra2 module or a homologue thereof. Any episome herein may be a plasmid.

Optionally, each plasimid comprises an expressible operon of a tra1 and/or tra2 module or a homologue thereof.

Optionally, each plasmid is comprised by a RK2 or R6K plasmid.

Optionally, each plasmid comprises an oriV of a RK2 or R6K plasmid, or a homologue thereof.

Optionally, each plasmid comprises an oriT of a RK2 or R6K plasmid, or a homologue thereof.

Optionally, the agent comprises one or more components of a CRISPR/Cas system that is operable in the target cell to cut a protospacer sequence comprised by the target cell, eg, wherein the protospacer sequence is comprised by the cell chromosome.

In an embodiment, the cutting herein kills the target cell. In an alternative, the cutting inhibits the growth or proliferation of the target cell.

Optionally, the agent encodes a guide RNA or crRNA of a CRISPR/Cas system that is operable with a Cas nuclease in the target cell to cut a protospacer sequence comprised by the target cell, eg, wherein the protospacer sequence is comprised by the cell chromosome.

In an example, the target cell is a Salmonella cell and the protospacer is comprised by a pipA, pipB, pipC, hilA, sicP, mart or sopB gene. In an example, the protospacer is comprised by a gene that is a homologue or orthologue of a Salmonella sicP, sseF, pipA, pipB, pipC, hilA, sicP, mart or sopB gene.

Optionally, each plasmid comprises a gene that encodes a product, wherein the product is essential for survival or proliferation of the carrier cell when in an environment that is devoid of the product, wherein the carrier cell chromosome does not comprise an expressible gene encoding the product and optionally the plasmid is the only episomal DNA comprised by the carrier cell that encodes the product. For example, the gene is selected from an aroA, argH, hisD, leuB, lysA, metB, proC, thrC, pheA, tyrA, trpC and pflA gene: or wherein the gene is an anti-toxin gene and optionally the first DNA encodes a cognate toxin.

For example, the carrier cell is an E. coli (eg, Nissle, F18 or S17 E. coli strain), Bacillus (eg, B. subtilis), Enterococcus or Lactobacillus cell.

Optionally, the carrier cell is a cell of a human, chicken pig, sheep, cow, fish (eg, catfish or salmon) or shellfish (eg, shrimp or lobster) commensal bacterial strain (eg, a commensal E. coli strain).

Optionally, each carrier cell is for administration to a microbiota of a human or animal subject for medical use.

For example, the medical use is for treating or preventing a disease disclosed herein. For example, the medical use is for treating or preventing a condition disclosed herein.

Optionally, the medical use is for the treatment or prevention of a disease or condition mediated by said target cells.

Optionally, the carrier cell(s) is(are) for administration to an animal for enhancing growth or weight of the animal.

In alternative, the administration is to a human for enhancing the growth or weight of the human. Optionally, the enhancing is not a medical therapy. Optionally, the enhancing is a medical therapy.

Optionally, the use comprises the administration of a plurality of carrier cells to a microbiota (eg, a gut microbiota) of the subject, wherein the microbiota comprises target cells and first DNA is transferred into target cells for expression therein to produce the antibacterial agent, thereby killing target cells in the subject or reducing the growth or proliferation of target cells.

For example a plant herein in any configuration or embodiment of the invention is selected from a tomato plant, a potato plant, a wheat plant, a corn plant, a maize plant, an apple tree, a bean-producing plant, a pea plant, a beetroot plant, a stone fruit plant, a barley plant, a hop plant and a grass. For example, the plant is a tree, eg, palm, a horse chestnut tree, a pine tree, an oak tree or a hardwood tree. For example the plant is a plant that produces fruit selected from strawberries, raspberries, blackberries, reducrrants, kiwi fruit, bananas, apples, apricots, avoocados, cherries, oranges, clementines, satsumas, grapefruits, plus, dates, figs, limes, lemons, melons, mangos, pears, olives or grapes. Optionally, the plant is a dicotyledon. Optionally, the plant is a flowering plant. Optionally, the plant is a monocotyledon.

In any configuration, embodiment or example herein, the target bacteria are P. syringae bacteria (eg, comprised by a plant). Pseudomonas syringae pv. syringae is a common plant-associated bacterium that causes diseases of both monocot and dicot plants worldwide. In an example the target bacteria are P. syringae bacteria of a pathovar selected from P. s. pv. aceris, P. s. pv. aptata, P. s. pv. atrofaciens, P. s. pv. dysoxylis, P. s. pv. japonica, P. s. pv. lapsa, P. s. pv. panici, P. s. pv. papulans, P. s. pv. pisi, P. s. pv. syringae and P. s. pv. morsprunorum.
P. s. pv. aceris attacks maple Acer species.
P. s. pv. actinidiae attacks kiwifruit Actinidia deliciosa.
P. s. pv. aesculi attacks horse chestnut Aesculus hippocastanum, causing bleeding canker.
P. s. pv. aptata attacks beets Beta vulgaris.
P. s. pv. atrofaciens attacks wheat Triticum aestivum.
P. s. pv. dysoxylis attacks the kohekohe tree Dysoxylum spectabile.
P. s. pv. japonica attacks barley Hordeum vulgare.
P. s. pv. lapsa attacks wheat Triticum aestivum.
P. s. pv. panici attacks Panicum grass species.
P. s. pv. papulans attacks crabapple Malus sylvestris species.
P. s. pv. phaseolicola causes halo blight of beans.
P. s. pv. pisi attacks peas Pisum sativum.
P. s. pv. syringae attacks Syringa, Prunus, and Phaseolus species.
P. s. pv. glycinea attacks soybean, causing bacterial blight of soybean.

In an example, the target bacteria are P. syringae selected from a serovar recited in a bullet point in the immediately preceding paragraph and the bacteria are comprised by a plant also mentioned in that bullet point.

In an example, the weight (ie, biomass) is dry weight. For example, the method is for increasing dry weight (eg, within 1 or 2 weeks of said administration). Optionally, the increase is an increase of at least 5, 10, 11, 12, 13, 14, 15, 16, 17, 18, 19 or 20% compared to a control plant of the same species or strain to which the administration if carrier cells has not taken place, wherein all plants are kept under the same environmental conditions. For example, such an increase is within 1, 2, 3, 4, 5, 6, or 8 weeks following the first administration of the carrier cells. In an example, the method is for increasing the dry weight of a leaf and/or fruit of the plant, such as a tomato plant.

In an example, the weight is wet weight. For example, the method is for increasing wet weight (eg, within 1 or 2 weeks of said administration). Optionally, the increase is an increase of at least 5, 10, 11, 12, 13, 14, 15, 16, 17, 18, 19 or 20% compared to a control plant of the same species or strain to which the administration if carrier cells has not taken place, wherein all plants are kept under the same environmental conditions. For example, such an increase is within 1, 2, 3, 4, 5, 6, or 8 weeks following the first administration of the carrier cells. In an example, the method is for increasing the dry weight of a leaf and/or fruit of the plant, such as a tomato plant.

For example, the microbiota is comprised by a leaf, trunk, root or stem of the plant.

The target bacteria (or target cell) may be comprised by a microbiota of a plant. In an example, the microbiota is comprised by a leaf. In an example, the microbiota is comprised by a xylem. In an example, the microbiota is comprised by a phloem. In an example, the microbiota is comprised by a root. In an example, the microbiota is comprised by a tuber. In an example, the microbiota is comprised by a bulb. In an example, the microbiota is comprised by a seed. In an example, the microbiota is comprised by an exocarp, epicarp, mesocarp or endocarp. In an example, the microbiota is comprised by a fruit, eg, a simple fruits: aggregate fruits: or multiple fruits. In an example, the microbiota is comprised by a seed or embryo, eg, by a seed coat: a seed leaf: cotyledons: or a radicle. In an example, the microbiota is comprised by a flower, eg, comprised by a peduncle: sepal: petals; stamen: filament: anther or pistil. In an example, the microbiota is comprised by a root: eg, a tap root system, or a fibrous root system. In an example, the microbiota is comprised by a leaf or leaves, eg, comprised by a leaf blade, petiole or stipule. In an example, the microbiota is comprised by a stem, eg, comprised by bark, epidermis, phloem, cambium, xylem or pith.

In an example “reducing a biofilm” comprises reducing the coverage area of the biofilm. In an example “reducing a biofilm” comprises reducing the proliferation of the biofilm. In an example “reducing a biofilm” comprises reducing the durability of the biofilm. In an example “reducing a biofilm” comprises reducing the spread of the biofilm (eg, in or on the subject, eg, spread to the environment containing the subject). The subject may be a human or animal.

For example, the biofilm is comprised by a lung of the subject, eg, wherein the target cells are Pseudomonas (eg, P. aeruginosa) cells. This may be useful wherein the subject is a human suffering from a lung disease or condition, such as pneumonia or cystic fibrosis.

For example, the biofilm is comprised by an animal or human organ disclosed herein. For example, the biofilm is comprised by a microbiota of a human or animal disclosed herein.

Optionally, said surface is a surface ex vivo, such as a surface comprised by a domestic or industrial apparatus or container.

Optionally, the target cells are comprised by a biofilm, eg, a biofilm as disclosed herein.

Optionally, the target bacteria are Salmonella, Pseudomonas, Escherichia, Klebsiella, Campylobacter, Helicobacter, Acinetobacter, Enterobacteriacea, Clostridium, Staphylococcus or Streptococcus bacteria. For example, the target bacteria are Salmonella enterica bacteria. For example, the target bacteria are selected from the group consisting of Salmonella enterica subsp. enterica, serovars Typhimurium, Enteritidis, Virchow, Montevideo, Hadar and Binza.

Optionally, the target bacteria are E. coli bacteria. For example, the target bacteria are enterohemorrhagic E. coli (EHEC), E. coli Serotype O157:H7 or Shiga-toxin producing E. coli (STEC)). In an example, the taraget bacteria are selected from
Shiga toxin-producing E. coli (STEC) (STEC may also be referred to as Verocytotoxin-producing E. coli (VTEC);
Enterohemorrhagic E. coli (EHEC) (this pathotype is the one most commonly heard about in the news in association with foodborne outbreaks);
Enterotoxigenic E. coli (ETEC);
Enteropathogenic E. coli (EPEC);
Enteroaggregative E. coli (EAEC);
Enteroinvasive E. coli (EIEC); and
Diffusely adherent E. coli (DAEC).

Enterohemorrhagic Escherichia coli (EHEC) serotype O157:H7 is a human pathogen responsible for outbreaks of bloody diarrhoea and haemolytic uremic syndrome (HUS) worldwide. Conventional antimicrobials trigger an SOS response in EHEC that promotes the release of the potent Shiga toxin that is responsible for much of the morbidity and mortality associated with EHEC infection. Cattle are a natural reservoir of EHEC, and approximately 75% of EHEC outbreaks are linked to the consumption of contaminated bovine-derived products. EHEC causes disease in humans but is asymptomatic in adult ruminants. Characteristics of E. coli serotype O157:H7 (EHEC) infection includes abdominal cramps and bloody diarrhoea, as well as the life-threatening complication haemolytic uremic syndrome (HUS). Currently there is a need for a treatment for EHEC infections (Goldwater and Bettelheim, 2012). The use of conventional antibiotics exacerbates Shiga toxin-mediated cytotoxicity. In an epidemiology study conducted by the Centers for Disease Control and Prevention, patients treated with antibiotics for EHEC enteritis had a higher risk of developing HUS (Slutsker et al., 1998). Additional studies support the contraindication of antibiotics in EHEC infection: children on antibiotic therapy for hemorrhagic colitis associated with EHEC had an increased chance of developing HUS (Wong et al., 2000; Zimmerhackl, 2000; Safdar et al., 2002; Tarr et al., 2005). Conventional antibiotics promote Shiga toxin production by enhancing the replication and expression of stx genes that are encoded within a chromosomally integrated lambdoid prophage genome. The approach of some configurations of present invention rely on nuclease cutting. Stx induction also promotes phage-mediated lysis of the EHEC cell envelope, allowing for the release and dissemination of Shiga toxin into the environment (Karch et al., 1999; Matsushiro et al., 1999; Wagner et al., 2002). Thus, advantageously, these configurations of the invention provide alternative means for treating EHEC in human and animal subjects. This is exemplified below with surprising results on the speed and duration of anti-EHEC action produced by nuclease action (as opposed to conventional antibiotic action).

In an example, the subject (eg, a human or animal) is suffering from or at risk of haemolytic uremic syndrome (HUS), eg, the subject is suffering from an E. coli infection, such as an EHEC E. coli infection.

There is provided:-

A pharmaceutical composition, livestock growth promoting composition, soil improver, herbicide, plant fertilizer, food or food ingredient sterilizing composition, dental composition, personal hygiene composition or disinfectant composition (eg, for domestic or industrial use) comprising a plurality of the carrier cells.

Herein, a carrier cell is, eg, a probiotic cell for administration to a human or animal subject. For example, the carrier cell is commensal in a microbiome (eg, gut or blood microbiome) of a human or animal subject, wherein the carrier is for administration to the subject. In an example, a carrier cell is a bacterial cell (and optionally the target cell is a bacterial cell). In an example, a carrier cell is an archaeal cell (and optionally the target cell is an archaeal cell)

Optionally, the carrier cell is a gram-positive bacterial cell and the target cell is a gram-positive bacterial cell.

Optionally, the carrier cell is a gram-positive bacterial cell and the target cell is a gram-negative bacterial cell.

Optionally, the carrier cell is a gram-negative bacterial cell and the target cell is a gram-positive bacterial cell.

Optionally, the carrier cell is a gram-negative bacterial cell and the target cell is a gram-negative bacterial cell.

Optionally, the carrier cell is a Bacillus bacterial cell and the target cell is a gram-positive bacterial cell.

Optionally, the carrier cell is a Bacillus bacterial cell and the target cell is a gram-negative bacterial cell.

Optionally, the carrier cell is a Bacillus bacterial cell and the target cell is a Salmonella bacterial cell.

Optionally, the carrier cell is a Bacillus bacterial cell and the target cell is an E. coli bacterial cell.

Optionally, the carrier cell is an E. coli bacterial cell and the target cell is a Pseudomonas bacterial cell.

Optionally, the carrier cell is an E. coli bacterial cell and the target cell is a gram-positive bacterial cell.

Optionally, the carrier cell is an E. coli bacterial cell and the target cell is a gram-netative bacterial cell.

Optionally, the carrier cell is an E. coli bacterial cell and the target cell is a Salmonella bacterial cell.

Optionally, the carrier cell is an E. coli bacterial cell and the target cell is an E. coli bacterial cell.

Optionally, the carrier cell is an E. coli bacterial cell and the target cell is a Pseudomonas bacterial cell.

A Bacillus cell herein is optionally a B. subtilis cell.

Optionally, the carrier cell is a probiotic or commensal E. coli bacterial cell for administration to a human or animal subject. Optionally, the carrier cell is a probiotic or commensal Bacillus bacterial cell for administration to a human or animal subject.

Herein, optionally the plasmid is a closed circular DNA.

In an embodiment, the plasmid DNA is dsDNA. In an embodiment, the plasmid DNA is ssDNA.

Optionally, the target cell is a Salmonella cell (eg, wherein the carrier cell is an E. coli cell), eg, a Salmonella enterica subsp. enterica, eg, a Salmonella enterica subsp. enterica serovar Typhimurium, Enteritidis, Virchow, Montevideo, Hadar or Binza.

For example, the target bacteria are selected from the group consisting of S. enterica; S. typhimurium; P. aeruginosa; E. coli; K. pneumoniae; C. jujeni; H. pylori; A. baumanii; C. difficile; S. aureus; S. pyogenes or S. thermophilus.

In an example, the target cell is a cell of a species that causes nosocomial infection in humans.

Optionally, the target cell is comprised by an animal (eg, poultry animal (such as chicken), swine, cow, fish (eg, catfish or salmon) or shellfish (eg, prawn or lobster)) microbiome. Optionally, the microbiome is a gut microbiome. For example, the target cell is a Salmonella cell comprised by a chicken gut biofilm. For example, the target cell is a Salmonella cell comprised by a chicken gut biofilm sample ex vivo.

In an embodiment, each plasmid comprises a bacterial oriV and/or an oriT. In an embodiment, each plasmid comprises and oriV and/or an oriT.

In an embodiment, the plasmid comprises an oriV and does not encode any replication protein (eg, pir or trfA) that is operable with the oriV to initiate replication of the plasmid.

In an example, the invention relates to a composition comprising a pluralty of carrier cells of the invention. Optionally, all of the carrier cells comprise identical said plasmids. Optionally, the plurality comprises a first sub-population of carrier cells (first cells) and a second sub-population of carrier cells (second cells) wherein the first cells comprise indentical first said plasmids and the second cells comprise indentical second said plasmids (which are different from the first plasmids of the first cells). For example, the first plasmids encode a first guide RNA or crRNA and the second plasmids encode a second guide RNA or crRNA, wherein the first guide RNA/crRNA is capable of hybridizing to a first protospacer sequence in first target cells; and the second guide RNA/crRNA is capable of hybridizing to a second protospacer sequence in second target cells, wherein the protospacers are different. Optionally, the first target cells are different from the second target cells. Optionally, the first target cells are of the same species or strain as the second target cells.

Alternatively, the first target cells are of species or strain that is different from the species or strain of the second target cells (in this way a cocktail of carrier cells is provided, eg, for administration to a human or animal or plant, to target and kill a plurality of target cells of different species or strains).

Optionally, the composition is comprised by a liquid (eg, an aqueous liquid or in water), the composition comprising the carrier cells at an amount of from 1×10³ to 1×10¹⁰ (eg, from 1×10⁴ to 1×10¹⁰; from 1×10⁴ to 1×10⁹; from 1×10⁴ to 1×10⁸; from 1×10⁴ to 1×10⁷; from 1×10³ to 1×10¹⁰; from 1×10³ to 1×10⁹; from 1×10³ to 1×10⁸; from 1×10³ to 1×10⁷; from 1×10⁵ to 1×10¹⁰; from 1×10⁵ to 1×10⁹; from 1×10⁵ to 1×10⁸; from 1×10⁵ to 1×10⁷; from 1×10⁶ to 1×10¹⁰; from 1×10⁶ to 1×10⁹; from 1×10⁶ to 1×10⁸; or from 1×10⁶ to 1×10⁷) cfu/ml. For example, the liquid is a beverage, such for human or animal consumption. For example, the beverage is a livestock beverage, eg, a poultry beverage (ie, a beverage for consumption by poultry, such as chicken).

In an example, the composition is a dietary (eg, dietary supplement) composition for consumption by humans or animals. In an example, the composition is a slimming composition for consumption by humans or animals. In an example, the composition is a growth promotion composition for consumption by humans or animals. In an example, the composition is a body buidling composition for consumption by humans. In an example, the composition is a probiotic composition for consumption by humans or animals. In an example, the composition is a biocidal composition for consumption by humans or animals. In an example, the composition is a pesticidal composition for consumption by humans or animals. In an example, the composition is a zoonosis control composition for consumption by animals.

In an example, the composition comprises vitamins in addition to the carrier cells. In an example, the composition comprises vitamin A, B (eg, B12), C, D, E and/or K in addition to the carrier cells. In an example, the composition comprises lipids in addition to the carrier cells. In an example, the composition comprises carbohydrates in addition to the carrier cells. In an example, the composition comprises proteins and/or amino acids in addition to the carrier cells. In an example, the composition comprises minerals in addition to the carrier cells. In an example, the composition comprises metal ions (eg, Mg²⁺, Cu²⁺ and/or Zn²⁺) in addition to the carrier cells. In an example, the composition comprises sodium ions, potassium ions, magnesium ions, calcium ions, manganese ions, iron ions, cobalt ions, copper ions, zinc ions and/or molybdenum ions.

In an example, the composition is a plant fertilizer composition. In an example, the composition is a herbicide. In an example, the composition is a pesticide composition for application to plants.

In any embodiment or example, where appropriate: The plants are, for example, crop plants. The plants are, for example, wheat. The plants are, for example, corn. The plants are, for example, maize. The plants are, for example, fruiting plants. The plants are, for example, vegetable plants. The plants are, for example, tomato plants. The plants are, for example, potato plants. The plants are, for example, grass plants. The plants are, for example, flowering plants. The plants are, for example, trees. The plants are, for example, shrubs.

In an example, the composition is for environmental application, wherein the environment is an outdoors environment (eg, application to a field or waterway or reservoir).

In an example, the composition is comprised by a food or food ingredient (eg, for human or animal consumption). In an example, the composition is comprised by a beverage or beverage ingredient (eg, for human or animal consumption).

In an example the target cell(s) are human biofilm cells, eg, wherein the biofilm is a gut, skin, lung, eye, nose, ear, gastrointestinal tract (GI tract), stomach, hair, kidney, urethra, bronchiole, oral cavity, mouth, liver, heart, anus, rectum, bladder, bowel, intestine, penis, vagina or scrotum biofilm. In an example the target cell(s) are animal biofilm cells, eg, wherein the biofilm is a gut, skin, lung, eye, nose, ear, gastrointestinal tract (GI tract), caecum, jejunum, ileum, colon, stomach, hair, feather, scales, kidney, urethra, bronchiole, oral cavity, mouth, liver, spleen, heart, anus, rectum, bladder, bowel, intestine, penis, vagina or scrotum biofilm. For example, the biofilm is a bird (eg, chicken) caecum biofilm. For example, the biofilm is a bird (eg, chicken) gastrointestinal tract (GI tract), caecum, jejunum, ileum, colon or stomach biofilm.

In an example, any method herein is ex vivo. In an example, a method herein is in vivo. In an example, a method herein is in vitro. In an example, a method herein is carried out in an environment, eg, in a domestic (such as in a house), industrial (such as in a factory) or agricultural environment (such as in a field). In an example, a method herein is carried out in or on a container: or on a surface.

In an example each plasmid comprises one or more components of a CRISPR/Cas system operable to perform protospacer cutting in the target cell (eg, wherein the protospacer comprises 10-20, 10-30, 10-40, 10-100, 12-15 or 12-20 consecutive nucleotides that are capable of hybridizing in the target cell with a crRNA or gRNA encoded by the NSI). For example, the system is a Type I, II, III, IV or V CRISPR/Cas system.

In an example, the or each plasmid encodes a Cas9 (and optionally a second, different, Cas, such as a Cas3, Cas9, Cpf1, Cas13a, Cas13b or Cas10); and/or a Cas3 (and optionally a second, different, Cas, such as a Cas3, Cas9, Cpf1, Cas13a, Cas13b or Cas10). In an example, the or each plasmid encodes a Cas selected from a Cas3, Cas9, Cpf1, Cas13a, Cas13b and Cas10. Additionally or alternatively, the plasmid encodes a guide RNA or crRNA or tracrRNA. For example, the guide RNA or crRNA or tracrRNA is cognate to (ie, operable with in the target cell) the first Cas.

In an example, a Cas herein is a Cas9. In an example, a Cas herein is a Cas3. The Cas may be identical to a Cas encoded by the target bacteria.

In an embodiment, each plasmid is a shuttle vector.

Optionally, the target cell is devoid of a functional endogenous CRISPR/Cas system before transfer therein of the plasmid, eg, wherein the plasmid comprises a component of an exogenous CRISPR/Cas system that is functional in the target cell and toxic to the target cell. An embodiment provides an antibacterial composition comprising a plurality of carrier cells of the invention, wherein each target cell is optionally according to this paragraph, for administration to a human or animal subject for medical use.

In an example, the composition of the invention is a herbicide, pesticide, insecticide, plant fertilizer or cleaning agent.

Optionally, target bacteria herein are comprised by a microbiome of the subject, eg, a gut microbiome. Alternatively, the microbiome is a skin, scalp, hair, eye, ear, oral, throat, lung, blood, rectal, anal, vaginal, scrotal, penile, nasal or tongue microbiome.

In an example the subject (eg, human or animal) is further administered a medicament simultaneously or sequentially with the carrier cell administration. In an example, the medicament is an antibiotic, antibody, immune checkpoint inhibitor (eg, an anti-PD-1, anti-PD-L1 or anti-CTLA4 antibody), adoptive cell therapy (eg, CAR-T therapy) or a vaccine.

In an embodiment, the plasmid encodes a guided nuclease, such as a Cas nuclease, TALEN, zinc finger nuclease or meganuclease. Thus, the toxic agent may comprise a guided nuclease, such as a Cas nuclease, TALEN, zinc finger nuclease or meganuclease. Optionally, the plasmid encodes a restriction nuclease that is capable of cutting the chromosome of the target cell.

Optionally, the composition is a pharmaceutical composition for use in medicine practised on a human or animal subject.

In an example, the animal is a livestock or companion pet animal (eg, a cow, pig, goat, sheep, horse, dog, cat or rabbit). In an example, the animal is an insect (an insect at any stage of its lifecycle, eg, egg, larva or pupa). In an example, the animal is a protozoan. In an example, the animal is a cephalopod.

Optionally, the composition is a herbicide, pesticide, food or beverage processing agent, food or beverage additive, petrochemical or fuel processing agent, water purifying agent, cosmetic additive, detergent additive or environmental (eg, soil) additive or cleaning agent.

For example the carrier bacteria are Lactobacillus (eg, L. reuteri or L. lactis), E. coli, Bacillus or Streptococcus (eg, S. thermophilus) bacteria. Usefully, the carrier can provide protection for the plasmid from the surrounding environment. The use of a carrier may be useful for oral administration or other routes where the carrier can provide protection for the plasmid from the acid stomach or other harsh environments in the subject. Furthermore, the carrier can be formulated into a beverage, for example, a probiotic drink, eg, an adapted Yakult (trademark), Actimel (trademark), Kevita (trademark), Activia (trademark), Jarrow (trademark) or similar drink for human consumption.

Optionally, the carrier cell(s) or composition are for administration to a human or animal subject for medical use, comprising killing target bacteria using the agent or expression product of the plasmid, wherein the target bacteria mediate as disease or condition in the subject. In an example, when the subject is a human, the subject is not an embryo. In an example, the carrier cells are probiotic in the subject.

Optionally, the environment is a microbiome of soil: a plant, part of a part (e.g., a leaf, fruit, vegetable or flower) or plant product (e.g., pulp): water; a waterway; a fluid: a foodstuff or ingredient thereof; a beverage or ingredient thereof; a medical device: a cosmetic; a detergent: blood; a bodily fluid: a medical apparatus: an industrial apparatus: an oil rig; a petrochemical processing, storage or transport apparatus: a vehicle or a container.

Optionally, the environment is an ex vivo bodily fluid (e.g., urine, blood, blood product, sweat, tears, sputum or spit), bodily solid (e.g., faeces) or tissue of a human or animal subject that has been administered the composition.

Optionally, the environment is an in vivo bodily fluid (e.g., urine, blood, blood product, sweat, tears, sputum or spit), bodily solid (e.g., faeces) or tissue of a human or animal subject that has been administered the composition.

In an embodiment, the plasmid is a phagemid or cloning vector (eg, a shuttle vector, eg, a pUC vector).

Optionally, the antibacterial agent comprises one or more components of a CRISPR/Cas system, eg, a DNA sequence encoding one or more components of Type I Cascade (eg, CasA).

Optionally, the agent comprises a DNA sequence encoding guided nuclease, such as a Cas nuclease, TALEN, zinc finger nuclease or meganuclease.

In an example, the carrier cell(s) or composition are comprised by a medical container, eg, a syringe, vial, IV bag, inhaler, eye dropper or nebulizer. In an example, the carrier cell(s) or composition are comprised by a sterile container. In an example, the carrier cell(s) or composition are comprised by a medically-compatible container. In an example, the carrier cell(s) or composition are comprised by a fermentation vessel, eg, a metal, glass or plastic vessel. In an example, the carrier cell(s) or composition are comprised by an agricultural apparatus. In an example, the carrier cell(s) or composition are comprised by food production or processing apparatus. In an example, the carrier cell(s) or composition are comprised by a horticultural apparatus. In an example, the carrier cell(s) or composition are comprised by a farming apparatus. In an example, the carrier cell(s) or composition are comprised by petrochemicals recovery or processing apparatus. In an example, the carrier cell(s) or composition are comprised by a distillation apparatus. In an example, the carrier cell(s) or composition are comprised by cell culture vessel (eg, having a capacity of at least 50, 100, 1000, 10000 or 100000 litres). Additionally or alternatively, the target cell(s) are comprised by any of these apparatus etc.

In an example, the carrier cell(s) or composition are comprised by a medicament, e,g in combination with instructions or a packaging label with directions to administer the medicament by oral, IV, subcutaneous, intranasal, intraocular, vaginal, topical, rectal or inhaled administration to a human or animal subject. In an example, the carrier cell(s) or composition are comprised by an oral medicament formulation. In an example, the carrier cell(s) or composition are comprised by an intranasal or ocular medicament formulation. In an example, the carrier cell(s) or composition are comprised by a personal hygiene composition (eg, shampoo, soap or deodorant) or cosmetic formulation. In an example, th the carrier cell(s) or composition are comprised by a detergent formulation. In an example, the carrier cell(s) or composition are comprised by a cleaning formulation, eg, for cleaning a medical or industrial device or apparatatus. In an example, the carrier cell(s) or composition are comprised by foodstuff, foodstuff ingredient or foodstuff processing agent. In an example, the carrier cell(s) or composition are comprised by beverage, beverage ingredient or beverage processing agent. In an example, the carrier cell(s) or composition are comprised by a medical bandage, fabric, plaster or swab. In an example, the carrier cell(s) or composition are comprised by a herbicide or pesticide. In an example, the carrier cell(s) or composition are comprised by an insecticide.

In an example, the CRISPR/Cas component(s) are component(s) of a Type I CRISPR/Cas system. In an example, the CRISPR/Cas component(s) are component(s) of a Type II CRISPR/Cas system. In an example, the CRISPR/Cas component(s) are component(s) of a Type III CRISPR/Cas system. In an example, the CRISPR/Cas component(s) are component(s) of a Type IV CRISPR/Cas system. In an example, the CRISPR/Cas component(s) are component(s) of a Type V CRISPR/Cas system. In an example, the CRISPR/Cas component(s) comprise a Cas9-encoding nucleotide sequence (eg, S. pyogenes Cas9, S. aureus Cas9 or S. thermophilus Cas9). In an example, the CRISPR/Cas component(s) comprise a Cas3-encoding nucleotide sequence (eg. E. coli Cas3, C. dificile Cas3 or Salmonella Cas3). In an example, the CRISPR/Cas component(s) comprise a Cpf-encoding nucleotide sequence. In an example, the CRISPR/Cas component(s) comprise a CasX-encoding nucleotide sequence. In an example, the CRISPR/Cas component(s) comprise a CasY-encoding nucleotide sequence.

In an example, each carrier cell encodes a CRISPR/Cas component from a nucleotide sequence (NSI) comprising a promoter that is operable in the target bacteria.

Optionally, target bacteria are gram negative bacteria (eg, a spirilla or vibrio). Optionally, target bacteria are gram positive bacteria. Optionally, target bacteria are mycoplasma, chlamydiae, spirochete or mycobacterium bacteria. Optionally, target bacteria are Streptococcus (eg, pyogenes or thermophilus). Optionally, target bacteria are Staphylococcus (eg, aureus, eg, MRSA). Optionally, target bacteria are E. coli (eg, 0157: H7), eg, wherein the Cas is encoded by the vecor or an endogenous target cell Cas nuclease (eg, Cas3) activity is de-repressed. Optionally, target bacteria are Pseudomonas (eg, syringae or aeruginosa). Optionally, target bacteria are Vibro (eg, cholerae (eg, O139) or vulnificus). Optionally, target bacteria are Neisseria (eg, gonnorrhoeae or meningitidis). Optionally, target bacteria are Bordetella (eg, pertussis). Optionally, target bacteria are Haemophilus (eg, influenzae). Optionally, target bacteria are Shigella (eg, dysenteriae). Optionally, target bacteria are Brucella (eg, abortus). Optionally, target bacteria are Francisella host. Optionally, target bacteria are Xanthomonas. Optionally, target bacteria are Agrobacterium. Optionally, target bacteria are Erwinia. Optionally, target bacteria are Legionella (eg, pneumophila). Optionally, target bacteria are Listeria (eg, monocytogenes). Optionally, target bacteria are Campylobacter (eg, jejuni). Optionally, target bacteria are Yersinia (eg, pestis). Optionally, target bacteria are Borelia (eg, burgdorferi). Optionally, target bacteria are Helicobacter (eg, pylori). Optionally, target bacteria are Clostridium (eg, dificile or botulinum). Optionally, target bacteria are Erlichia (eg, chaffeensis). Optionally, target bacteria are Salmonella (eg, typhi or enterica, eg, serotype typhimurium, eg, DT 104). Optionally, target bacteria are Chlamydia (eg, pneumoniae). Optionally, target bacteria are Parachlamydia host. Optionally, target bacteria are Corynebacterium (eg, amycolatum). Optionally, target bacteria are Klebsiella (eg, pneumoniae). Optionally, target bacteria are Enterococcus (eg, faecalis or faecim, eg, linezolid-resistant). Optionally, target bacteria are Acinetobacter (eg, baumannii, eg, multiple drug resistant).

Further examples of target cells are as follows:-
1. Optionally the target bacteria are Staphylococcus aureus cells, eg, resistant to an antibiotic selected from methicillin, vancomycin, linezolid, daptomycin, quinupristin, dalfopristin and teicoplanin.
2 Optionally the target bacteria are Pseudomonas aeuroginosa cells, eg, resistant to an antibiotic selected from cephalosporins (eg, ceftazidime), carbapenems (eg, imipenem or meropenem), fluoroquinolones, aminoglycosides (eg, gentamicin or tobramycin) and colistin.
3 Optionally the target bacteria are Klebsiella (eg, pneumoniae) cells, eg, resistant to carbapenem.
4. Optionally the target bacteria are Streptoccocus (eg, thermophilus, pneumoniae or pyogenes) cells, eg, resistant to an antibiotic selected from erythromycin, clindamycin, beta-lactam, macrolide, amoxicillin, azithromycin and penicillin.
5. Optionally the target bacteria are Salmonella (eg, serotype Typhi) cells, eg, resistant to an antibiotic selected from ceftriaxone, azithromycin and ciprofloxacin.
6. Optionally the target bacteria are Shigella cells, eg, resistant to an antibiotic selected from ciprofloxacin and azithromycin.
7. Optionally the target bacteria are Mycobacterium tuberculosis cells, eg, resistant to an antibiotic selected from Resistance to isoniazid (INH), rifampicin (RMP), fluoroquinolone, amikacin, kanamycin and capreomycin and azithromycin.
8 Optionally the target bacteria are Enterococcus cells, eg, resistant to vancomycin.
9 Optionally the target bacteria are Enterobacteriaceae cells, eg, resistant to an antibiotic selected from a cephalosporin and carbapenem.
10. Optionally the target bacteria are E. coli cells, eg, resistant to an antibiotic selected from trimethoprim, itrofurantoin, cefalexin and amoxicillin.
11. Optionally the target bacteria are Clostridium (eg, dificile) cells, eg, resistant to an antibiotic selected from fluoroquinolone antibiotic and carbapenem.
12. Optionally the target bacteria are Neisseria gonnorrhoea cells, eg, resistant to an antibiotic selected from cefixime (eg, an oral cephalosporin), ceftriaxone (an injectable cephalosporin), azithromycin and tetracycline.
13. Optionally the target bacteria are Acinetoebacter baumannii cells, eg, resistant to an antibiotic selected from beta-lactam, meropenem and a carbapenem.
14. Optionally the target bacteria are Campylobacter (eg, jejuni) cells, eg, resistant to an antibiotic selected from ciprofloxacin and azithromycin.
15. Optionally, the target cell(s) produce Beta (β)-lactamase (eg, ESBL-producing E. coli or ESBL-producing Klebsiella).
16. Optionally, the target cell(s) are bacterial cells that are resistant to an antibiotic recited in any one of examples 1 to 14.

In an example, the target cell(s) is a cell of a species selected from Shigella, E. coli, Salmonella, Serratia, Klebsiella, Yersinia, Pseudomonas and Enterobacter.

Optionally, the composition comprises carrier cells that are each or in combination capable of conjugative transfer of first DNAs into target cells of species selected from two or more of Shigella, E coli, Salmonella, Serratia, Klebsiella, Yersinia, Pseudomonas and Enterobacter.

In an example, the reduction in growth or proliferation of target cells is at least 50, 60, 70, 80, 90 or 95%. Optionally, the composition or carrier cell(s) are administered simultaneously or sequentially with an an antibiotic that is toxic to the target cells. For example, the antibiotic can be any antibiotic disclosed herein.

Optioanlly, the expression of the agent is under the control of an inducible promoter that is operable in the target cell. Optioanlly, the expression of the agent is under the control of a constitutive promoterthat is operable in the target cell.

In embodiments, the plasmid contains a screenable or selectable marker gene. For example, the selectable marker gene is an antibiotic resistance gene.

The carrier bacteria can be bacteria of a species or genus as follows. For example, the species is found in warm-blooded animals (eg, livestock vertebrates). For example, the species is found in humans. For example, the species is found in plants. Preferably, non-pathogenic bacteria that colonize the non-sterile parts of the human or animal body (e.g., skin, digestive tract, urogenital region, mouth, nasal passages, throat and upper airway, ears and eyes) are utilized as carrier cells, and in an example the methodology of the invention is used to combat a target cell bacterial infection of such a part of the body of a human or animal. In another embodiment, the infection is systemic infection. Examples of particularly preferred carrier bacterial species include, but are not limited to: non-pathogenic strains of Escherichia coli (E. coli F18, S17 and E. coli strain Nissle), various species of Lactobacillus (such as L. casei, L. plantarum, L. paracasei, L. acidophilus, L. fermentum, L. zeae and L. gasseri), or other nonpathogenic or probiotic skin- or GI colonizing bacteria such as Lactococcus, Bifidobacteria, Eubacteria, and bacterial mini-cells, which are anucleoid cells destined to die but still capable of transferring plasmids (see: e.g., Adler et al., Proc. Natl. Acad. Sci. USA 57; 321-326, 1970; Frazer and Curtiss III, Current Topics in Microbiology and Immunology 69: 1-84, 1975: U.S. Pat. No. 4,968,619 to Curtiss III). In some embodiments, the target recipient cells are pathogenic bacteria comprised by a human, animal or plant, eg, on the skin or in the digestive tract, urogenital region, mouth, nasal passage, throat and upper airway, eye(s) and ear(s). Of particular interest for targeting and eradication are pathogenic strains of Pseudomonas aeruginosa, Escherichia coli, Staphylococcus pneumoniae and other species, Enterobacter spp., Enterococcus spp. and Mycobacterium tuberculosis.

The present invention finds use with a wide array of settings or environments, eg, in therapeutic, agricultural, or other settings, including, but not limited to, those described in U.S. Pat. Nos. 6,271,359, 6,261,842, 6,221,582, 6,153,381, 6,106,854, and 5,627,275. Others are also discussed herein, and still others will be readily apparent to those of skill in the art.

A single carrier bacterial strain might harbor more than one type of such plasmid (eg, differing in the antibacterial agent that they encode). Further, in another example two or more different carrier bacterial strains, each containing one or more such plasmids, may be combined for a multi-target effect, ie, for killing two or more different target species or strains, or for killing the cells of the same species or strain of target cell.

The present invention finds utility for treatment of humans and in a variety of veterinary, agronomic, horticultural and food processing applications. For human and veterinary use, and depending on the cell population or tissue targeted for protection, the following modes of administration of the carrier bacteria of the invention are contemplated: topical, oral, nasal, ocular, aural, pulmonary (e.g., via an inhaler), ophthalmic, rectal, urogenital, subcutaneous, intraperitoneal and intravenous. The bacteria may be supplied as a pharmaceutical composition, in a delivery vehicle suitable for the mode of administration selected for the patient being treated. The term “patient” or “subject” as used here may refer to humans or animals (animals being particularly useful as models for clinical efficacy of a particular donor strain, for example, or being farmed or livestock animals). Commercially-relevant animals are chicken, turkey, duck, catfish, salmon, cod, herring, lobster, shrimp, prawns, cows, sheep, goats, pigs, goats, geese or rabbits.

For example, to deliver the carrier bacteria to the gastrointestinal tract or to the nasal passages, the preferred mode of administration may be by oral ingestion or nasal aerosol, or by feeding (alone or incorporated into the subject's feed or food and/or beverage, such as drinking water). In this regard, the carrier cells may be comprised by a food of livestock (or farmed or companion animal), eg, the carrier bacteria are comprised by a feed additive for livestock. Alternatively, the additive is a beverage (eg, water) additive for livestock. It should be noted that probiotic bacteria, such as Lactobacillus acidophilus, are sold as gel capsules containing a lyophilized mixture of bacterial cells and a solid support such as mannitol. When the gel capsule is ingested with liquid, the lyophilized cells are re-hydrated and become viable, colonogenic bacteria. Thus, in a similar fashion, carrier bacterial cells of the present invention can be supplied as a powdered, lyophilized preparation in a gel capsule, or in bulk, eg, for sprinkling onto food or beverages. The re-hydrated, viable bacterial cells will then populate and/or colomze sites throughout the upper and/or lower gastrointestinal system, and thereafter come into contact with the target bacteria.

For topical applications, the carrier bacteria may be formulated as an ointment or cream to be spread on the affected skin surface. Ointment or cream formulations are also suitable for rectal or vaginal delivery, along with other standard formulations, such as suppositories. The appropriate formulations for topical, vaginal or rectal administration are well known to medicinal chemists. The present invention will be of particular utility for topical or mucosal administrations to treat a variety of bacterial infections or bacterially related undesirable conditions. Some representative examples of these uses include treatment of (1) conjunctivitis, caused by Haemophilus sp., and corneal ulcers, caused by Pseudomonas aeruginosa: (2) otititis externa, caused by Pseudomonas aeruginosa: (3) chronic sinusitis, caused by many Gram-positive cocci and Gram-negative rods, or for general decontamination of bronchii: (4) cystic fibrosis, associated with Pseudomonas aeruginosa: (5) enteritis, caused by Helicobacter pylori (eg, to treat or prevent gastric ulcers), Escherichia coli, Salmonella typhimurium, Campylobacter or Shigella sp.; (6) open wounds, such as surgical or non-surgical, eg, as a prophylactic measure: (7) burns to eliminate Pseudomonas aeruginosa or other Gram-negative pathogens: (8) acne, eg, caused by Propionobacter acnes: (9) nose or skin infection, eg, caused by metlncillin resistant Staphylococcus aureus (MSRA): (10) body odor, eg, caused by Gram-positive anaerobic bacteria (i.e., use of carrier cells in deodorants): (11) bacterial vaginosis, eg, associated with Gardnerella vaginalis or other anaerobes; and (12) gingivitis and/or tooth decay caused by various organisms.

In one example, the target cells are E. coli cells and the disease or condition to be treated or prevented in a human is a uterine tract infection or a ventilator associated infection, eg, pneumonia, sepsis, septicaemia or HUS.

In other embodiments, the carrier cells of the present invention find application in the treatment of surfaces for the removal or attenuation of unwanted target bacteria, for example use in a method of treating such a surface or an environment comprising target bacteria, wherein the method comprises contacting the surface or environment with carrier bacteria of the invention, allowing conjugative transfer of the first DNA of the invention from the carrier to the target bacteria, and allowing the antibacterial agent to kill target cells. For example, surfaces that may be used in invasive treatments such as surgery, catheterization and the like may be treated to prevent infection of a subject by bacterial contaminants on the surface. It is contemplated that the methods and compositions of the present invention may be used to treat numerous surfaces, objects, materials and the like (e.g., medical or first aid equipment, nursery and kitchen equipment and surfaces) to control bacterial contamination thereon.

Pharmaceutical preparations or other compositions comprising the carrier bacteria may be formulated in dosage unit form for ease of administration and uniformity of dosage. Dosage unit form, as used herein, refers to a physically discrete unit of the pharmaceutical preparation appropriate for the patient or plant or environment or surface undergoing treatment. Each dosage should contain a quantity of the carrier bacteria calculated to produce the desired antibacterial effect in association with the selected carrier. Procedures for determining the appropriate dosage unit are well known to those skilled in the art. Dosage units may be proportionately increased or decreased based on the weight of a patient, plant, surface or environment. Appropriate concentrations for achieving eradication of pathogenic target cells (eg, comprised by a tissue of the patient) may be determined by dosage concentration curve calculations, as known in the art.

Other uses for the carrier bacteria of the invention are also contemplated. These include a variety agricultural, horticultural, environmental and food processing applications. For example, in agriculture and horticulture, various plant pathogenic bacteria may be targeted in order to minimize plant disease. One example of a plant pathogen suitable for targeting is Erwinia (eg, E. amylovora, the causal agent of fire blight). Similar strategies may be utilized to reduce or prevent wilting of cut flowers. For veterinary or animal farming, the carrier cells of the invention may be incorporated into animal feed (chicken, swine, poultry, goat, sheep, fish, shellfish or cattle feed) to reduce bio-burden or to eliminate certain pathogenic organisms (e.g., Salmonella, such as in chicken, turkey or other poultry). In other embodiments, the invention may be applied on meat or other foods to eliminate unwanted or pathogenic bacteria (e.g., E. coli O157:H7 on meat, or Proteus spp., one cause of “fishy odour” on seafood).

Environmental utilities comprise, for example, engineering carrier bacteria, eg, Bacillus thurengiensis and one of its conjugative plasmids, to deliver and conditionally express an insecticidal agent in addition to or instead of an antibacterial agent (e.g., for the control of mosquitos that disseminate malaria or West Nile virus). In such applications, as well as in the agricultural and horticultural or other applications described above, formulation of the carrier bacteria as solutions, aerosols, or gel capsules are contemplated.

As used herein, the term “carrier cell” may include dividing and/or non-dividing bacterial cells (minicells and maxicells), or conditionally non-functional cells.

In an example the plasmid is an engineered RK2 plasmid (ie, a RK2 plasmid that has been modified by recombinant DNA technology or a progeny of such a modified plasmid). Plasmid RK2 is a promiscuous plasmid that can replicate in 29 (and probably many more) gram-negative species (Guiney and Lanka, 1989, p 27-54. In C. M. Thomas (ed) Promiscous plasmids in gram-negative bacteria. London, Ltd London United Kingdom.). Plasmid RK2 is a 60-kb self-transmissible plasmid with a complete nucleotide sequence known (Pansegrau et al., 1994, J. Mol. Biol. 239, 623-663). A minimal replicon derived from this large plasmid has been obtained that is devoid of all its genes except for a trfA gene, that encodes plasmid's Rep protein called TrfA, and an origin of vegetative replication oriV For a review of RK2 replication and its control by TrfA protein, see Helinski et al., 1996 (In Escherichia coli and Salmonella Cellular and Molecular Biology, Vol. 2 (ed. F. Neidhardt, et al., 2295-2324, ASM Press, Washington D.C.).

In an example the plasmid is an engineered R6K plasmid (ie, a R6K plasmid that has been modified by recombinant DNA technology or a progeny of such a modified plasmid).

The present invention is optionally for an industrial or domestic use, or is used in a method for such use. For example, it is for or used in agriculture, oil or petroleum industry, food or drink industry, clothing industry, packaging industry, electronics industry, computer industry, environmental industry, chemical industry, acorspace industry, automotive industry, biotechnology industry, medical industry, healthcare industry, dentistry industry, energy industry, consumer products industry, pharmaceutical industry, mining industry, cleaning industry, forestry industry, fishing industry, leisure industry, recycling industry, cosmetics industry, plastics industry, pulp or paper industry, textile industry, clothing industry, leather or suede or animal hide industry, tobacco industry or steel industry.

The present invention is optionally for use in an industry or the environment is an industrial environment, wherein the industry is an industry of a field selected from the group consisting of the medical and healthcare: pharmaceutical: human food: animal food: plant fertilizers: beverage: dairy; meat processing: agriculture: livestock farming: poultry farming: fish and shellfish farming; veterinary: oil: gas: petrochemical: water treatment: sewage treatment: packaging: electronics and computer: personal healthcare and toiletries: cosmetics: dental: non-medical dental: ophthalmic: non-medical ophthalmic: mineral mining and processing: metals mining and processing: quarrying; aviation: automotive: rail: shipping: space: environmental: soil treatment: pulp and paper: clothing manufacture: dyes: printing: adhesives: air treatment: solvents: biodefence: vitamin supplements: cold storage: fibre retting and production: biotechnology: chemical: industrial cleaning products: domestic cleaning products: soaps and detergents: consumer products: forestry: fishing: leisure: recycling; plastics: hide, leather and suede: waste management: funeral and undertaking: fuel: building: energy; steel; and tobacco industry fields.

In an example, the plasmid comprises a CRISPR array that targets target bacteria, wherein the array comprises one, or two or more different spacers (eg, 2, 3, 4, 5, 6, 7, 8, 9, 10, 20, 30, 40, 50 or more spacers) for targeting the genome of target bacteria.

In an example, the target bacteria are comprised by an environment as follows. In an example, the environment is a microbiome of a human, eg, the oral cavity microbiome or gut microbiome or the bloodstream. In an example, the environment is not an environment in or on a human. In an example, the environment is not an environment in or on a non-human animal. In an embodiment, the environment is an air environment. In an embodiment, the environment is an agricultural environment. In an embodiment, the environment is an oil or petroleum recovery environment, eg, an oil or petroleum field or well. In an example, the environment is an environment in or on a foodstuff or beverage for human or non-human animal consumption. In an example, the environment is a maritimeenvironment, eg, in seawater or on a boat (eg, in ship or boat ballast water).

In an example, the environment is a a human or animal microbiome (eg, gut, vaginal, scalp, armpit, skin or oral cavity microbiome). In an example, the target bacteria are comprised by a human or animal microbiome (eg, gut, vaginal, scalp, armpit, skin or oral cavity microbiome).

In an example, the carrier bacteria or composition of the invention are administered intranasally, topically or orally to a human or non-human animal, or is for such administration. The skilled person aiming to treat a microbiome of the human or animal will be able to determine the best route of administration, depending upon the microbiome of interest. For example, when the microbiome is a gut microbiome, administration can be intranasally or orally. When the microbiome is a scalp or armpit microbiome, administration can be topically. When the microbiome is in the mouth or throat, the administration can be orally.

In an example, the environment is harboured by a beverage or water (eg, a waterway or drinking water for human consumption) or soil. The water is optionally in a heating, cooling or industrial system, or in a drinking water storage container.

In an example, the carrier and/or target bacteraia are Firmicutes selected from Anaerotruncus, Acetanaerobacterium, Acetitomaculum, Acetivibrio, Anaerococcus, Anaerofilum, Anaerosinus, Anaerostipes, Anaerovorax, Butyrivibrio, Clostridium, Capracoccus, Dehalobacter, Dialister, Dorea, Enterococcus, Ethanoligenens, Faecalibacterium, Fusobacterium, Gracilibacter, Guggenheimella, Hespellia, Lachnobacterium, Lachnospira, Lactobacillus, Leuconostoc, Megamonas, Moryella, Mitsuokella, Oribacterium, Oxobacter, Papillibacter, Proprionispira, Pseudobutyrivibrio, Pseudoramibacter, Roseburia, Ruminococcus, Sarcina, Seinonella, Shuttleworthia, Sporobacter, Sporobacterium, Streptococcus, Subdoligranulum, Syntrophococcus, Thermobacillus, Turibacter and Weisella.

In an example, the carrier bacteria, composition, use or method is for reducing pathogenic infections or for re-balancing gut or oral biofilm eg, for treating or preventing obesity or disease in a human or animal: or for treating or preventing a GI condition (such as Crohn's disease, IBD or colitis). For example, the DNA, carrier bacteria, composition, use or method is for knocking-down Salmomnella, Campylobacter, Erwinia, Xanthomonous, Edwardsiella, Pseudomonas, Klebsiella, Pectobacterium, Clostridium dificile or E. coli bacteria in a gut biofilm of a human or animal or a plant, preferably in a human or animal.

In an example, the animal is a chicken, eg, and the target bacteria are Salmomnella or Campylobacter. In an example, the animal is a fish (eg, catfish or salmon) or shellfish (eg, prawn or lobster), eg, and the target bacteria are Edwardsiella. In an example, the plant is a potato plant and, eg, the target bacteria are Pectobacterium. In an example, the plant is a cabbage plant and, eg, the target bacteria are Xanthomonous (eg, X. campestris). In an example, the plant is a marijuana plant and, eg, the targt bacteria are Pseudomonas (eg, P. cannabina or P. amygdali), Agrobacterium (eg, A. tumefaciens) or Xanthomonas (eg, X. campestris). In an example, the plant is a hemp plant and, eg, the targt bacteria are are Pseudomonas (eg, P. cannabina or P. amygdali), Agrobacterium (eg, A. tumefaciens) or Xanthomonas (eg, X. campestris).

In an example, the disease or condition is a cancer, inflammatory or autoimmune disease or condition, eg, obesity, diabetes IBD, a GI tract condition or an oral cavity condition.

Optionally, the environment is comprised by, or the target bacteria are comprised by, a gut biofilm, skin biofilm, oral cavity biofilm, throat biofilm, hair biofilm, armpit biofilm, vaginal biofilm, rectal biofilm, anal biofilm, ocular biofilm, nasal biofilm, tongue biofilm, lung biofilm, liver biofilm, kidney biofilm, genital biofilm, penile biofilm, scrotal biofilm, mammary gland biofilm, ear biofilm, urethra biofilm, labial biofilm, organ biofilm or dental biofilm. Optionally, the environment is comprised by, or the target bacteria are comprised by, a plant (eg, a tobacco, crop plant, fruit plant, vegetable plant or tobacco, eg on the surface of a plant or contained in a plant) or by an environment (eg, soil or water or a waterway or acqueous liquid).

In an example, the carrier cell(s) or composition is for treating a disease or condition in an animal or human, wherein the disease or condition. In an example, the disease or condition is caused by or mediated by an infection of target cells comprised by the subject or patient. In an example, the disease or condition is associated with an infection of target cells comprised by the subject or patient. In an example, a symptom of the disease or condition is an infection of target cells comprised by the subject or patient.

Optionally, the disease or condition of a human or animal subject is selected from
(a) A neurodegenerative disease or condition;
(b) A brain disease or condition;
(c) A CNS disease or condition;
(d) Memory loss or impairment;
(e) A heart or cardiovascular disease or condition, eg, heart attack, stroke or atrial fibrillation;
(f) A liver disease or condition;
(g) A kidney disease or condition, eg, chronic kidney disease (CKD);
(h) A pancreas disease or condition;
(i) A lung disease or condition, eg, cystic fibrosis or COPD;
(j) A gastrointestinal disease or condition;
(k) A throat or oral cavity disease or condition;
(l) An ocular disease or condition;
(m) A genital disease or condition, eg, a vaginal, labial, penile or scrotal disease or condition;
(n) A sexually-transmissible disease or condition, eg, gonorrhea, HIV infection, syphilis or Chlamydia infection;
(o) An ear disease or condition;
(p) A skin disease or condition;
(q) A heart disease or condition;
(r) A nasal disease or condition
(s) A haematological disease or condition, eg, anaemia, eg, anaemia of chronic disease or cancer;
(t) A viral infection;
(u) A pathogenic bacterial infection;
(v) A cancer;
(w) An autoimmune disease or condition, eg, SLE;
(x) An inflammatory disease or condition, eg, rheumatoid arthritis, psoriasis, eczema, asthma, ulcerative colitis, colitis, Crohn's disease or IBD;
(y) Autism;
(z) ADHD;
(aa) Bipolar disorder;
(bb) ALS [Amyotrophic Lateral Sclerosis];
(cc) Osteoarthritis;
(dd) A congenital or development defect or condition;
(ee) Miscarriage;
(ff) A blood clotting condition;
(gg) Bronchitis;
(hh) Dry or wet AMD;
(ii) Neovascularisation (eg, of a tumour or in the eye);
(jj) Common cold;
(kk) Epilepsy;
(ll) Fibrosis, eg, liver or lung fibrosis;
(mm) A fungal disease or condition, eg, thrush;
(nn) A metabolic disease or condition, eg, obesity, anorexia, diabetes, Type I or Type II diabetes.
(oo) Ulcer(s), eg, gastric ulceration or skin ulceration;
(pp) Dry skin;
(qq) Sjogren's syndrome;
(rr) Cytokine storm;
(ss) Deafness, hearing loss or impairment;
(tt) Slow or fast metabolism (ie, slower or faster than average for the weight, sex and age of the subject);
(uu) Conception disorder, eg, infertility or low fertility;
(vv) Jaundice;
(ww) Skin rash;
(xx) Kawasaki Disease;
(yy) Lyme Disease;
(zz) An allergy, eg, a nut, grass, pollen, dust mite, cat or dog fur or dander allergy;
(aaa) Malaria, typhoid fever, tuberculosis or cholera;
(bbb) Depression;
(ccc) Mental retardation;
(ddd) Microcephaly;
(eee) Malnutrition;
(fff) Conjunctivitis;
(ggg) Pneumonia;
(hhh) Pulmonary embolism;
(iii) Pulmonary hypertension;
(jjj) A bone disorder;
(kkk) Sepsis or septic shock;
(lll) Sinusitus;
(mmm) Stress (eg, occupational stress);
(nnn) Thalassaemia, anaemia, von Willebrand Disease, or haemophilia;
(ooo) Shingles or cold sore;
(ppp) Menstruation;
(qqq) Low sperm count.

### Neurodegenerative or CNS Diseases or Conditions for Treatment or Prevention by the Invention

In an example, the neurodegenerative or CNS disease or condition is selected from the group consisting of Alzheimer disease, geriopsychosis, Down syndrome, Parkinson's disease, Creutzfeldt-jakob disease, diabetic neuropathy, Parkinson syndrome, Huntington's disease, Machado-Joseph disease, amyotrophic lateral sclerosis, diabetic neuropathy, and Creutzfeldt Creutzfeldt- Jakob disease. For example, the disease is Alzheimer disease. For example, the disease is Parkinson syndrome.

In an example, wherein the method of the invention is practised on a human or animal subject for treating a CNS or neurodegenerative disease or condition, the method causes downregulation of Treg cells in the subject, thereby promoting entry of systemic monocyte-derived macrophages and/or Treg cells across the choroid plexus into the brain of the subject, whereby the disease or condition (eg, Alzheimer's disease) is treated, prevented or progression thereof is reduced. In an embodiment the method causes an increase of IFN-gamma in the CNS system (eg, in the brain and/or CSF) of the subject. In an example, the method restores nerve fibre and//or reduces the progression of nerve fibre damage. In an example, the method restores nerve myelin and//or reduces the progression of nerve myelin damage. In an example, the method of the invention treats or prevents a disease or condition disclosed in WO2015136541 and/or the method can be used with any method disclosed in WO2015136541 (the disclosure of this document is incorporated by reference herein in its entirety, eg, for providing disclosure of such methods, diseases, conditions and potential therapeutic agents that can be administered to the subject for effecting treatement and/or prevention of CNS and neurodegenerative diseases and conditions, eg, agents such as immune checkpoint inhibitors, eg, anti-PD-1, anti-PD-L1, anti-TIM3 or other antibodies disclosed therein).

### Cancers for Treatment or Prevention by the Method

Cancers that may be treated include tumours that are not vascularized, or not substantially vascularized, as well as vascularized tumours. The cancers may comprise non-solid tumours (such as haematological tumours, for example, leukaemias and lymphomas) or may comprise solid tumours. Types of cancers to be treated with the invention include, but are not limited to, carcinoma, blastoma, and sarcoma, and certain leukaemia or lymphoid malignancies, benign and malignant tumours, and malignancies e.g., sarcomas, carcinomas, and melanomas. Adult tumours/cancers and paediatric tumours/cancers are also included.

Haematologic cancers are cancers of the blood or bone marrow. Examples of haematological (or haematogenous) cancers include leukaemias, including acute leukaemias (such as acute lymphocytic leukaemia, acute myelocytic leukaemia, acute myelogenous leukaemia and myeloblasts, promyeiocytic, myelomonocytic, monocytic and erythroleukaemia), chronic leukaemias (such as chronic myelocytic (granulocytic) leukaemia, chronic myelogenous leukaemia, and chronic lymphocytic leukaemia), polycythemia vera, lymphoma, Hodgkin's disease, non-Hodgkin's lymphoma (indolent and high grade forms), multiple myeloma, Waldenstrom's macroglobulinemia, heavy chain disease, myeiodysplastic syndrome, hairy cell leukaemia and myelodysplasia.

Solid tumours are abnormal masses of tissue that usually do not contain cysts or liquid areas. Solid tumours can be benign or malignant. Different types of solid tumours are named for the type of cells that form them (such as sarcomas, carcinomas, and lymphomas). Examples of solid tumours, such as sarcomas and carcinomas, include fibrosarcoma, myxosarcoma, liposarcoma, chondrosarcoma, osteosarcoma, and other sarcomas, synovioma, mesothelioma, Ewing's tumour, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous eel! carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumour, cervical cancer, testicular tumour, seminoma, bladder carcinoma, melanoma, and CNS tumours (such as a glioma (such as brainstem glioma and mixed gliomas), glioblastoma (also known as glioblastoma multiforme) astrocytoma, CNS lymphoma, germinoma, medu!loblastoma, Schwannoma craniopharyogioma, ependymoma, pineaioma, hemangioblastoma, acoustic neuroma, oligodendroglioma, menangioma, neuroblastoma, retinoblastoma and brain metastases).

### Autoimmune Diseases for Treatment or Prevention by the Method

1. Acute Disseminated Encephalomyelitis (ADEM)
2. Acute necrotizing hemorrhagic leukoencephalitis
3. Addison's disease
4. Agammaglobulinemia
5. Alopecia areata
6. Amyloidosis
7. Ankylosing spondylitis
8. Anti-GBM/Anti-TBM nephritis
9. Antiphospholipid syndrome (APS)
10. Autoimmune angioedema
11. Autoimmune aplastic anemia
12. Autoimmune dysautonomia
13. Autoimmune hepatitis
14. Autoimmune hyperlipidemia
15. Autoimmune immunodeficiency
16. Autoimmune inner ear disease (AIED)
17. Autoimmune myocarditis
18. Autoimmune oophoritis
19. Autoimmune pancreatitis
20. Autoimmune retinopathy
21. Autoimmune thrombocytopeniarpura (ATP)
22. Autoimmune thyroid disease
23. Autoimmune urticaria
24. Axonal & neuronal neuropathies
25. Balo disease
26. Behcet's disease
27. Bullous pemphigoid
28. Cardiomyopathy
29. Castleman disease
30. Celiac disease
31. Chagas disease
32. Chronic fatigue syndrome
33. Chronic inflammatory demyelinating polyneuropathy (CIDP)
34. Chronic recurrent multifocal ostomyelitis (CRMO)
35. Churg-Strauss syndrome
36. Cicatricial pemphigoid/benign mucosal pemphigoid
37. Crohn's disease
38. Cogans syndrome
39. Cold agglutinin disease
40. Congenital heart block
41. Coxsackie myocarditis
42. CREST disease
43. Essential mixed cryoglobulinemia
44. Demyelinating neuropathies
45. Dermatitis herpetiformis
46. Dermatomyositis
47. Devic's disease (neuromyelitis optica)
48. Discoid lupus
49. Dressler's syndrome
50. Endometriosis
51. Eosinophilic esophagitis
52. Eosinophilic fasciitis
53. Erythema nodosum
54. Experimental allergic encephalomyelitis
55. Evans syndrome
56. Fibromyalgia
57. Fibrosing alveolitis
58. Giant cell arteritis (temporal arteritis)
59. Giant cell myocarditis
60. Glomerulonephritis
61. Goodpasture's syndrome
62. Granulomatosis with Polyangiitis (GPA) (formerly called Wegener's Granulomatosis)
63. Graves' disease
64. Guillain-Barre syndrome
65. Hashimoto's encephalitis
66. Hashimoto's thyroiditis
67. Hemolytic anemia
68. Henoch-Schonlein purpura
69. Herpes gestationis
70. Hypogammaglobulinemia
71. Idiopathic thrombocytopenia purpura (ITP)
72. IgA nephropathy
73. IgG4-related sclerosing disease
74. Immunoregulatory lipoproteins
75. Inclusion body myositis
76. Interstitial cystitis
77. Juvenile arthritis
78. Juvenile diabetes (Type 1 diabetes)
79. Juvenile myositis
80. Kawasaki syndrome
81. Lambert-Eaton syndrome
82. Leukocytoclastic vasculitis
83. Lichen planus
84. Lichen sclerosus
85. Ligneous conjunctivitis
86. Linear IgA disease (LAD)
87. Lupus (SLE)
88. Lyme disease, chronic
89. Meniere's disease
90. Microscopic polyangiitis
91. Mixed connective tissue disease (MCTD)
92. Mooren's ulcer
93. Mucha-Habermann disease
94. Multiple sclerosis
95. Myasthenia gravis
96. Myositis
97. Narcolepsy
98. Neuromyelitis optica (Devic's)
99. Neutropenia
100. Ocular cicatricial pemphigoid
101. Optic neuritis
102. Palindromic rheumatism
103. PANDAS (Pediatric Autoimmune Neuropsychiatric Disorders Associated with Streptococcus)
104. Paraneoplastic cerebellar degeneration
105. Paroxysmal nocturnal hemoglobinuria (PNH)
106 Parry Romberg syndrome
107. Parsonnage-Turner syndrome
108. Pars planitis (peripheral uveitis)
109 Pemphigus
110 Peripheral neuropathy
111. Perivenous encephalomyelitis
112. Pernicious anemia
113. POEMS syndrome
114. Polyarteritis nodosa
115. Type I, II, & III autoimmune polyglandular syndromes
116. Polymyalgia rheumatica
117. Polymyositis
118. Postmyocardial infarction syndrome
119. Postpericardiotomy syndrome
120. Progesterone dermatitis
121. Primary biliary cirrhosis
122. Primary sclerosing cholangitis
123. Psoriasis
124. Psoriatic arthritis
125. Idiopathic pulmonary fibrosis
126. Pyoderma gangrenosum
127. Pure red cell aplasia
128. Raynauds phenomenon
129. Reactive Arthritis
130. Reflex sympathetic dystrophy
131. Reiter's syndrome
132. Relapsing polychondritis
133. Restless legs syndrome
134. Retroperitoneal fibrosis
135. Rheumatic fever
136. Rheumatoid arthritis
137. Sarcoidosis
138. Schmidt syndrome
139. Scleritis
140. Scleroderma
141. Sjogren's syndrome
142. Sperm & testicular autoimmunity
143. Stiff person syndrome
144. Subacute bacterial endocarditis (SBE)
145. Susac's syndrome
146. Sympathetic ophthalmia
147. Takayasu's arteritis
148. Temporal arteritis/Giant cell arteritis
149 Thrombocytopeniarpura (TTP)
150. Tolosa-Hunt syndrome
151. Transverse myelitis
152. Type 1 diabetes
153. Ulcerative colitis
154 Undifferentiated connective tissue disease (UCTD)
155 Uveitis
156. Vasculitis
157 Vesiculobullous dermatosis
158 Vitiligo
159. Wegener's granulomatosis (now termed Granulomatosis with Polyangiitis (GPA).

### Inflammatory Diseases for Treatment or Prevention by the Method

1. Alzheimer
2. ankylosing spondylitis
3. arthritis (osteoarthritis, rheumatoid arthritis (RA), psoriatic arthritis)
4. asthma
5. atherosclerosis
6. Crohn's disease
7. colitis
8. dermatitis
9. diverticulitis
10. fibromyalgia
11. hepatitis
12. irritable bowel syndrome (IBS)
13. systemic lupus erythematous (SLE)
14. nephritis
15. Parkinson's disease
16. ulcerative colitis.

For example, the composition comprising carrier cells is an animal feed and/or beverage (eg, mixed in drinking water). When supplied in a beverage, the system, component or agent may be comprised by carrier bacteria, wherein the carrier bacteria are comprised in the beverage at an amount of from 1×10³ to 1×10¹⁰ (eg, from 1×10⁴ to 1×10¹⁰; from 1×10⁴ to 1×10⁹; from 1×10⁴ to 1×10⁸; from 1×10⁴ to 1×10⁷; from 1×10³ to 1×10¹⁰; from 1×10³ to 1×10⁹; from 1×10³ to 1×10⁸; from 1×10³ to 1×10⁷; from 1×10⁵ to 1×10¹⁰; from 1×10⁵ to 1×10⁹; from 1×10⁵ to 1×10⁸; from 1×10⁵ to 1×10⁷; from 1×10⁶ to 1×10¹⁰; from 1×10⁶ to 1×10⁹; from 1×10⁶ to 1×10⁸; or from 1×10⁶ to 1×10⁷) cfu/ml. When supplied in a beverage, the system, component or agent may be comprised by carrier bacteria, wherein the carrier bacteria are comprised in the beverage at an amount of at least 1×10⁸ cfu/ml, eg, wherein the animal is a poultry bird, such as a chicken.

Optionally, the guided nuclease is any guided nuclease disclosed herein, eg, a Cas, TALEN, meganuclease or a zinc finger nuclease. In an example, the component is a crRNA or guide RNA that is operable in target cells with a cognate Cas nuclease. The Cas nuclease can be any Cas nuclease disclosed herein. The Cas nuclease may be an endogenous Cas of the target cells or may be encoded by an exogenous nucleic acid that is administered to the animal.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications and all US equivalent patent applications and patents are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps

The term “or combinations thereof” or similar as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### EXAMPLES

Pseudomonas syringae pv. tomato str. DC3000, used in the Examples, has the complete genome sequence of which has GenBank accession number AE016853.1, the entire sequence of which is incorporated herein by reference.

P. fluoroscens strain 896 (pfu 896), used in the Examples, has the complete genome sequence of GenBank accession number CABVIN000000000.1, the entire sequence of which is incorporated herein by reference. P fluoroscens strain 887 (pfu 887), used in the Examples, has the complete genome sequence of GenBank accession number CABVIQ000000000.1, the entire sequence of which is incorporated herein by reference.

### Example 1: Effective Delivery, Killing & Maintenance of Antibacterial Agent Using Conjugation to Bacteria with RND-Efflux Pumps

### Aim of the Study

This study was performed to evaluate the efficacy of a conjugation-delivered anti-P. syringae antibacterial CRISPR/Cas agent, when used as a protective product to selectively target and kill P. syrinage pv. tomato, DC3000 strain (Pto DC3000) in the cv. Moneymaker variety of tomato plants. Herein, we refer to the agent as a CRISPR Guided Biotic™ (GB™). The Pto DC3000 comprised genes encoding RND efflux pumps, including genes PSPTO_0820 and PSPTO_4977.

### Background/Scope

P. syringae pv. tomato (Pto) is a pathogen of tomato plants. The disease caused by Pto is characterised by bacterial specks, which start to appear on the leaves of young transplants. If the disease is left unmanaged in the developing plants, it causes death of the plants. This has been reported as a major cause of concern in the United States¹ and more recently in Italy², where the yield of tomato crops has been severely affected by the bacterial speck disease. We investigated CRISPR Guided Biotic™ (GB™) technology to target the pathogen on or in plants to protect or manage the disease and prevent the loss of yield.

Our GB™ technology against Pto DC3000, was based on the CRISPR/Cas system, carried on a conjugative plasmid vector. The active GB™ vector encoded a Cas nuclease and cognate crRNA, with crRNA spacers targeting two conserved and essential genes in the genome of P. syringae DC3000. Both genes are chromosomally located on the genome of Pto DC3000. A control GB™ vector contained all of the other components of the active GB™ vector but didn't encode the crRNA. For the delivery of GB™ vectors to the target bacteria in plants, we selected a non-pathogenic bacterium which forms part of the normal microbiota of plants as well as being present in soil and water. Two strains were developed and compared for the delivery of GB™ vectors. To enable the conjugative transfer of GB™ vectors, the conjugative plasmid (p)RP4 was transformed into the delivery strains (ie, into the carrier cells). The pRP4 is a 60 kb plasmid, which is also incorporated in the genome of the E. coli S17^{3,4}. Finally, the control and active GB™ vectors were transformed into the delivery strains.

### Materials and Methods

The Moneymaker tomato plants were sown and two weeks after sowing seedlings were transplanted into 9 cm pots. The experiment was performed in a contaminant level 2 plant room. The plants were allowed to grow for seven weeks, before the start of the experiment. The strains used in this study and their characteristics are as follows:-

| | |
|---|---|
| Plant control | Delivery strain 1 containing conjugative pRP4 and GB ™ control vector. |
| GB ™ 1 | The pRP4 encoded a tetracycline marker of selection and GB ™ vector |
| | encoded a gentamicin marker for selection. |
| Plant active | Delivery strain 1 containing conjugative, pRP4 and GB ™ active vector |
| GB ™ 1 | encoding cRNA targeting two conserved and essential genes. The pRP4 |
| | encoded a tetracycline marker of selection and GB ™ vector encoded a |
| | gentamicin marker for selection. |
| Plant control | Delivery strain 2 containing conjugative pRP4 and GB ™ control |
| GB ™ 2 | vector. The pRP4 encoded a tetracycline marker of selection and GB ™ |
| | vector encoded a gentamicin marker for selection. |
| Plant active | Delivery strain 2 containing conjugative, pRP4 and GB ™ active vector |
| GB ™ 2 | encoding cRNA targeting two conserved and essential genes. The pRP4 |
| | encoded a tetracycline marker of selection and GB ™ vector encoded a |
| | gentamicin marker for selection. |
| Pto DC3000 | P. syringae pv. syringae DC3000 wild type strain, with chromosomally |
| | encoded rifampicin marker for selection. |

The GB™ control and active strains were inoculated in Lysogeny (L) media (Sigma-Aldrich, UK), containing 12.5 μg/mL tetracycline and 25 μg/mL of gentamicin. These cultures and Pto DC3000 in L media were allowed to grow shaking at 28° C., overnight. After the overnight incubation, the cultures were centrifuged at 4000×g for 15 minutes. After centrifugation, the supernatant was discarded. The pellet was gently suspended into 10 mM MgCl₂ and centrifuged again as stated above. The pellet was washed three times by suspending in fresh 10 mM MgCl₂ and centrifugation each time. Finally, the OD₆₀₀ₙₘ of each culture was measured using the spectrophotometer. The OD₆₀₀ₙₘ of each GB™ active and control strain was adjusted to 0.3 in 10 mM MgCl₂ containing 0.04% Silwet. The Pto DC3000 was adjusted to 0.1 in 10 mM MgCl₂ containing 0.04% Silwet™. The following treatments were applied in this study.

Treatment combinations and the number of plants per treatment, used in this study:-

| | Number of plants | | |
|---|---|---|---|
| | Biological | Biological | Biological |
| Treatment | replicate 1 | replicate 2 | replicate 3 |
| Plant control | 3 | 3 | 3 |
| GB ™ 1 + Pto | | | |
| DC3000 | | | |
| Plant active | 3 | 3 | 3 |
| GB ™ 1 + Pto | | | |
| DC3000 | | | |
| Plant control | 3 | 3 | 3 |
| GB ™ 2 + Pto | | | |
| DC3000 | | | |
| Plant active | 3 | 3 | 3 |
| GB ™ 2 + Pto | | | |
| DC3000 | | | |
| Pto DC3000- | 2 | 3 | 3 |
| disease control | | | |
| MgCl₂ negative | 2 | 3 | 3 |
| control | | | |

In the first experiment, for homogeneity of coverage of the plants the ‘dip inoculation method’ was used for the application of the respective GB™ treatment. For dip inoculation, the plant pot was carefully inverted and dipped in the treatment contained in a 1 L beaker. The pathogen, Pto DC3000 control and 10 mM MgCl₂ containing 0.04% Silwet, as negative control was sprayed on the plants. For spray inoculation, plastic plant water spray bottles were used. The spray bottles had jet and mist control to ensure uniform spraying on plant. Both the GB™ treatment and Pto DC3000 were applied as single applications. For the remaining biological replicates, both the GB™ treatments and controls were sprayed on the plants. The treatment was allowed to dry for 2-3 hours (hrs). After this time, Pto DC3000 was sprayed on all the plants, except for the plants in the negative control group in which the plants were only sprayed with 10 mM MgCl₂ containing 0.04% Silwet. The leaf disc samples were made and processed as follows: After the plants were dry, two leaf discs were collected from each of the three separate leaves per plant, by using a cork borer No. 2 (area=0.125 cm²). The leaf discs were ground in 100 μL volume of 10 mM MgCl₂. The leaf disc extracts were serially diluted in 10 mM MgCl₂ and 10 μL spot of each dilution was plated in duplicate on the L media agar plates containing 50 μg/mL of rifampicin, which selects for Pto DC3000 and 25 μg/mL of Nystatin, which was used as an anti-fungal agent. The leaf disc extracts from each plant was obtained after 24, 48, 72 hrs and 7 days and were processed as described above. The plates were incubated at 28° C., for 48 hrs. The bacterial colonies were counted and CFU/cm² was calculated⁵. Percentage (%) reduction in pathogen (Pto DC3000) load was calculated as: (CFU/cm² plant control GB™-CFU/cm² plant active GB™)/CFU/cm² plant control GB™*100. The log reduction in pathogen (Pto DC3000) load was calculated as: log (CFU/cm² plant control GB™)-log (CFU/cm² plant active GB™).

### Results and Discussion

The conjugative carrier bacteria with GB™ vector was applied on the tomato plants. These plants were then exposed to infection with Pto DC3000 by spray application on the plants, except for the first biological replicate in which dip inoculation method was used. Any surviving Pto DC3000 after treatment with the GB™ was enumerated and compared to the control groups.

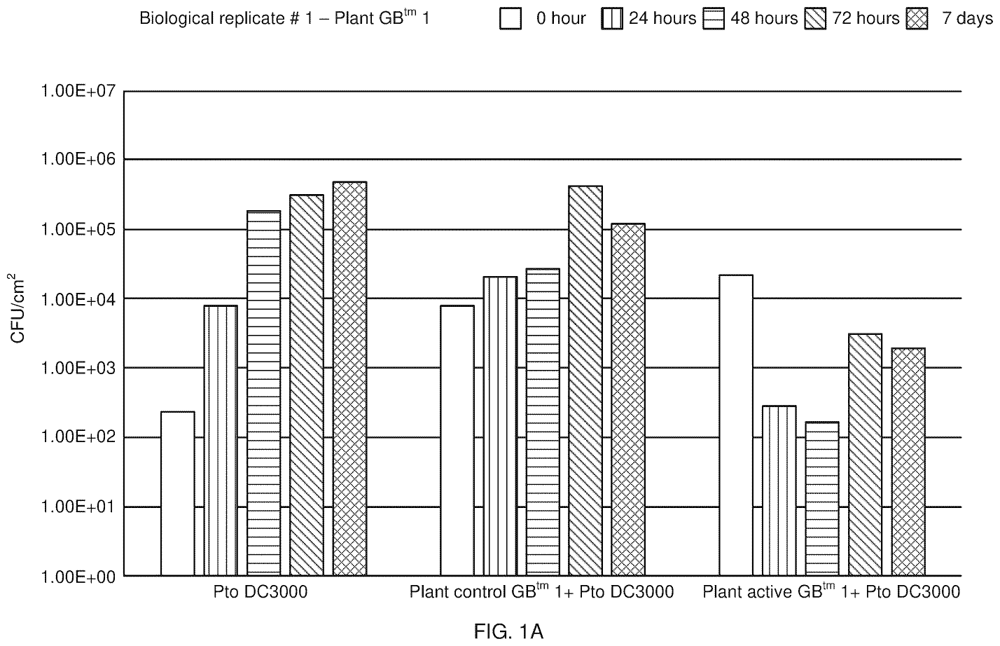

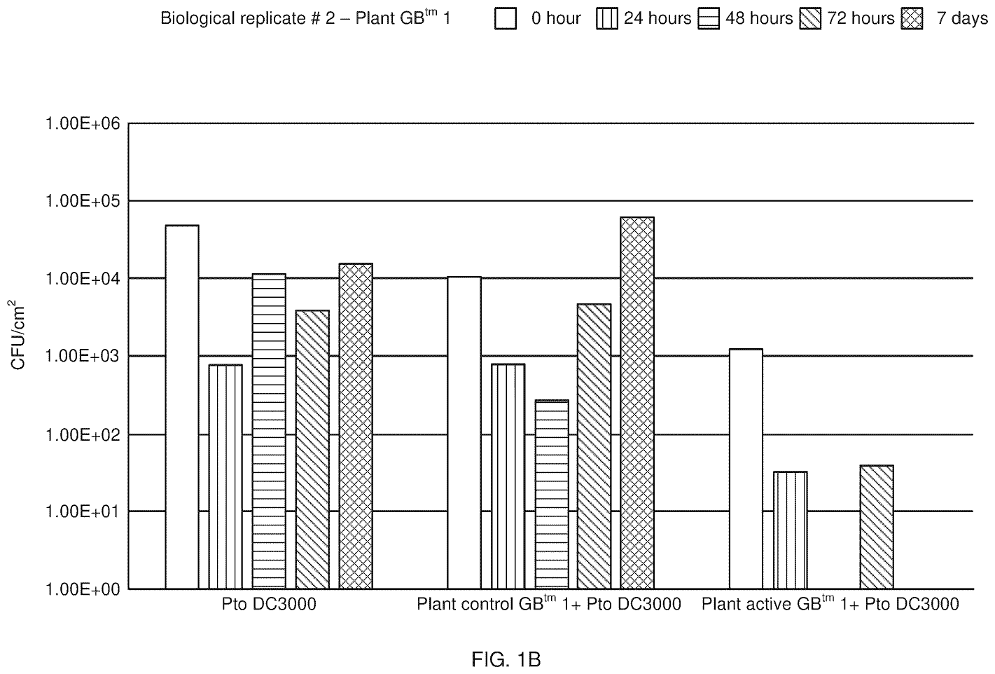

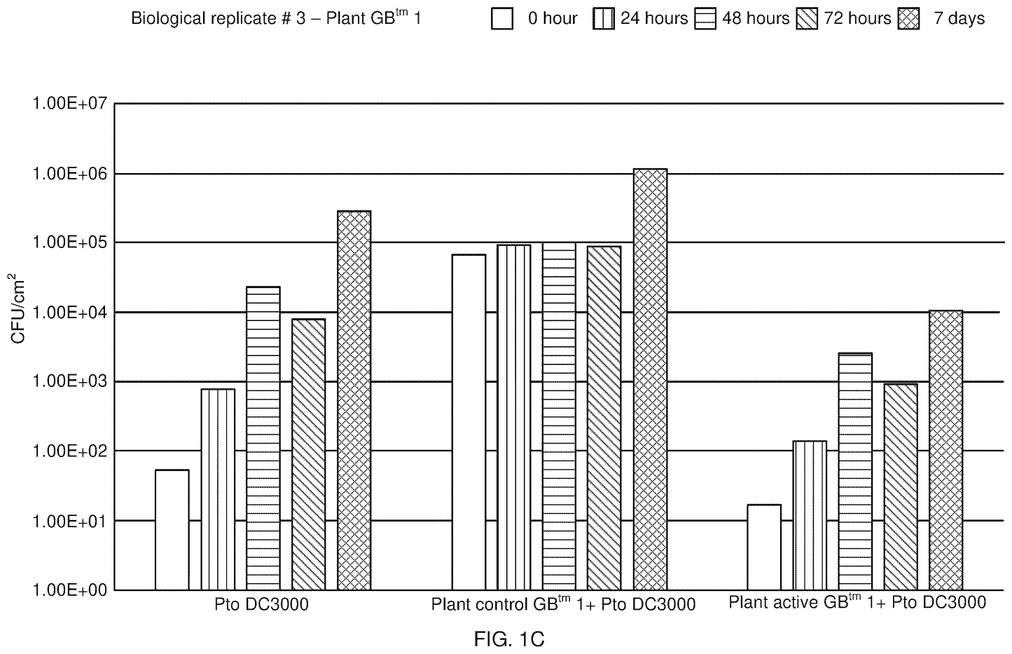

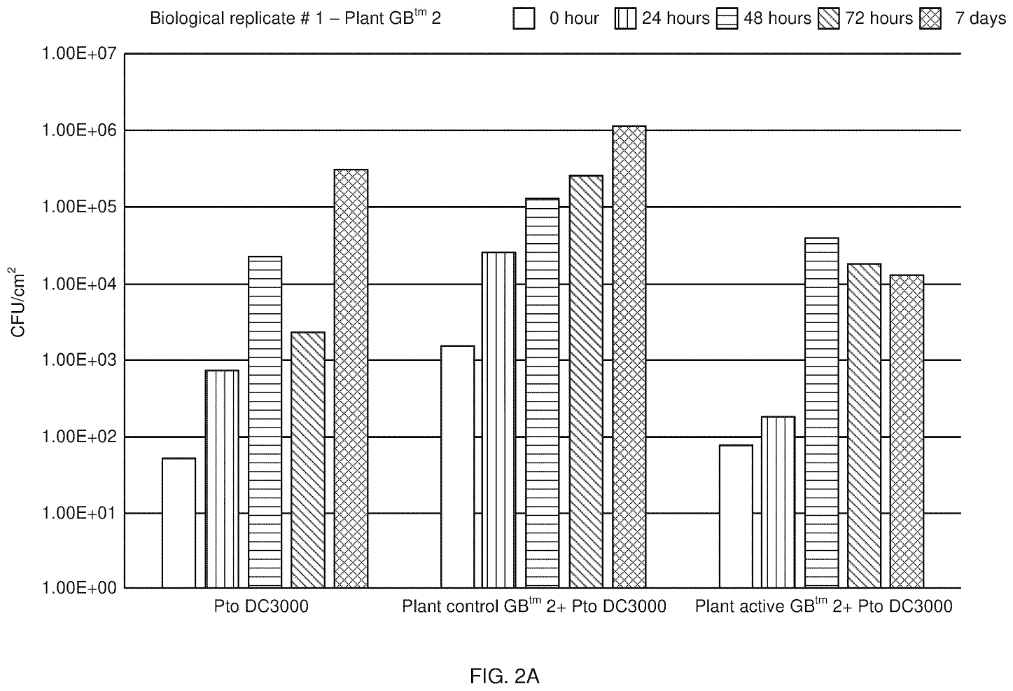

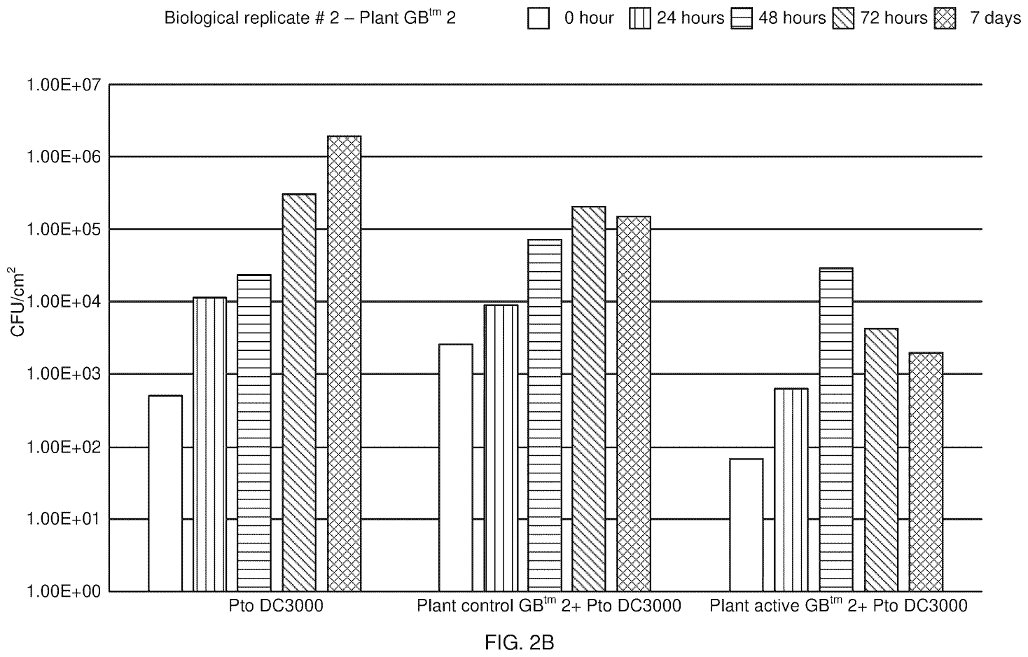

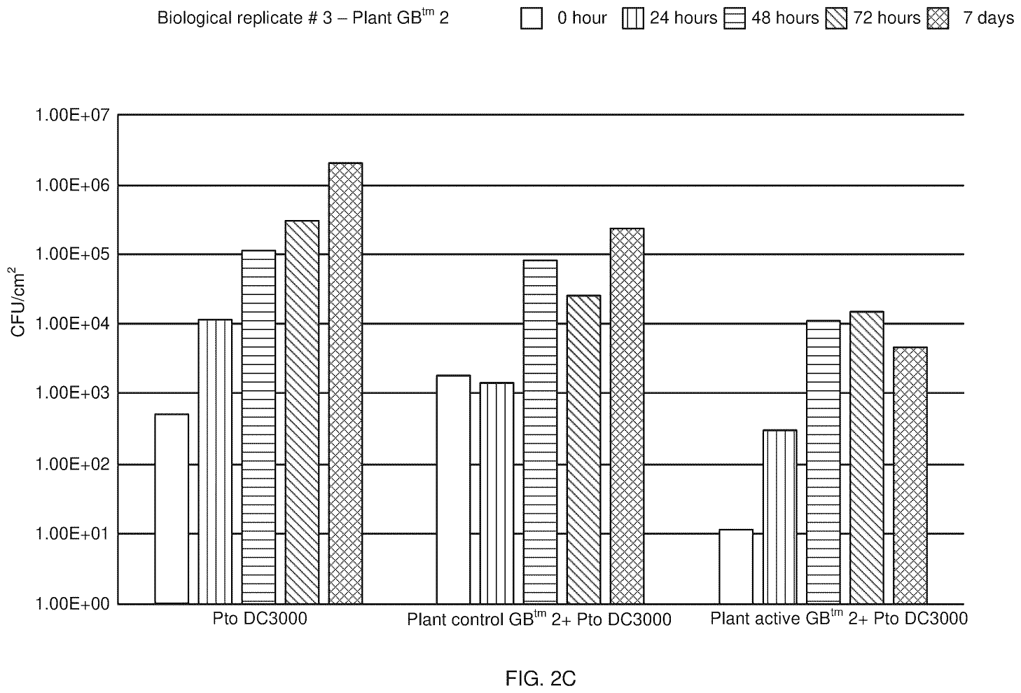

The protection assays showed that Pto DC3000 applied as the positive control for disease on average achieved a 2.5 log increase in CFU/cm², 7 days post-infection (FIGS. 1 and 2). The treatment with active GB™ 1 showed an overall 1.7 log reduction in CFU/cm², compared with the control GB™ 1 (FIG. 1 and table 8). A similar trend in the CFU/cm² reduction of Pto DC3000 was observed for the active GB™ 2 with an overall 1.2 log reduction in the pathogen load on plants, compared with the control GB™ 2 (FIG. 2 and Table 8).

Although, there was a variation in the log reduction values in the load of Pto DC3000, per timepoint between the active GB™ compared with the control GB™ (FIGS. 1 and 2), the active GB™ successfully reduced the pathogen load in all cases.

Conclusively, the reduction in the number of Pto DC3000 as a result of the application of active GB™ using conjugation showed the guided biotic is effective in killing on plants. Thus, surprisingly we were able to effectively achieve delivery of an antibacterial agent into the target cells using conjugation, despite the presence of RND efflux pumps. The delivered agent, furthermore, was advantageously retained sufficiently to enable killing by measurable and meaningful amounts. This study also suggests that active conjugative GB™ survives on the plants for up to a week, acting as a bactericidal against Pto DC3000, keeping the bacterial burden down. In this respect, see Table 9: we calculated the percentage reduction in the bacterial load referred to as the % kill by active GB at day 1 post-treatment (beginning of the experiment) and also at day 7 (end of the experiment). The % kill at each time point (i.e. day 1 and day 7) was compared with the non-active (or control) GB. The difference in the % kill between day 7 and day 1 was calculated as the average difference in % kill for triplicate experiments. for each of the two delivery strains used (Pfu 896 and Pfu 887). As seen in Table 9, the killing effect was surprisingly durable and maintained or even increased at day 7.

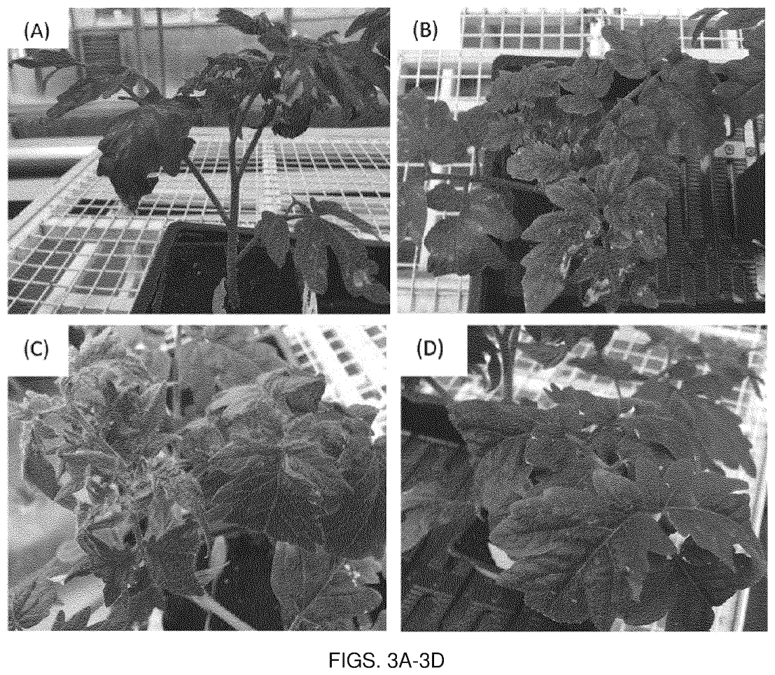

The set of plants treated with Pto DC3000 only and the ones treated with the control GB™ and then exposed to Pto DC3000 developed characteristic bacterial specks, and signs of chlorosis and necrosis of the infected leaves were also visible, after 7 days of the treatment. FIGS. 3A and 3B are representative images of plants treated with the plant control GB™ 1 and then sprayed with Pto DC3000, showing successful infection by Pto DC3000. As a comparison, the plants treated with the plant active GB™ 1 showed infection control and healthier plants (FIG. 3C). In this case, only localized symptoms of the disease were observed on some leaves (FIG. 3D), which may represent the areas of the leaves where the plant active GB™ 1 did not come in contact with the active pathogen, Pto DC3000 or the plant active GB™ 1 did not survive or replicate in all foliar parts of the plant. Generally. plants treated according to the invention were healthier compared with the control.

### Example 2: Bioninformatics Analysis to Determine Bacterial Species & Strains with Gene Orthologues & Homologues

In order to determine the homologues and orthologues for the PSPTO_0477 and PSPTO_0820, the nucleotide sequences of these genes were used to perform the BLASTN search, using the NCBI online search tool (https://blast.ncbi.nlm.nih.gov/Blast.cgi). The searches were performed against the databases available on 27 Apr. 2020. The homologues to the genes PSPTO_0477 and PSPTO_0820 were found (Tables 3 to 6) by performing the BLASTN search against the NCBI's standard non-redundent nucleotide (nr/nt) collection database and the top 100 hits are reported. A BLASTN search against the standard non-redundent nucleotide (nr/nt) collection database excluding the Pseudomonadales provided the orthologues of PSPTO_0477 and PSPTO_0820 in the non-Pseudomonas species (Tables 4 and 6). For PSPTO_0820, this search achieved several best hits with the percentage (%) sequence identity in the range of 80-82% of the length of the query sequence in the range of 97-98%. The top best hit for each species is reported (Table 6).

### Example 3 Determination of Desirable Carrier Strain Characteristics

Data mining of the genome sequence of a collection of P. fluorescens strains showed the presence or absence of genes or operons involved in the natural product pathways in these strains (Reference 6, FIG. 2). We determined that motile P. fluorescens carrier strains used in our study performed well; non-motile strains were found to be poor performers. The gene encoding for PepI is present in the genome of only the motile strains used in our study and absent from the non-motile strains. PepI has a role in the RiPP (ribosomally synthesized and post-translationally modified peptides) pathway (Reference 7). PepI encodes for a 69 amino acid product which provides immunity against Pep5, a lantibiotic (an antimicrobial peptide) produced by gram positive Staphylococcus epidermidis(Reference 8). We also determined that another gene, the gene encoding for the chitinase class I exoenzyme, is present in most motile strains studied and is absent from the non-motile strains. This enzyme is produced by Pseudomonas aeruginosa and it breaks down the polymer chitin, which is present in the cell wall of algae and fungi into the extracellular environment (Reference 9). The presence of these two genes exclusively in the motile strains suggests the role of these genes in the improved performance in pathogen control, advantage in colonisation and thereby the yield gain in planta.

### REFERENCES

¹Jones J B, Mc Carter S M, and Gitaitis R. (1981). Pseudomonas syringae pv. syringae with a leaf spot disease of tomato transplants in southern Georgia. Phytopathology. 71:1281-1285.
²Garibaldi A, Minuto A, Scortichini M and Gullino, M. (2007). First Report of Syringae Leaf Spot Caused by Pseudomonas syringae pv. syringae on Tomato in Italy. Plant disease. 91:1518. doi: 10.1094/PDIS-91-11-1518B
³Pansegrau W, Lanka E, Barth P T, Figurski D H, Guiney D G, Haas D, Helinski D R, Schwab H, Stanisich V A, and Thomas C M. (1994). Complete nucleotide sequence of Birmingham IncP plasmids: compilation and comparative analysis. J. Mol. Biol. 239:623-663.
⁴Strand T A, Lale R, Degnes K F, Lando M and Valla S. (2014). A new and improved host-independent plasmid system for RK2-based conjugal transfer. PLoS One. 9(3):e90372. doi: 10.1371/journal.pone.0090372. PMID: 24595202: PMCID: PMC3940858.
⁵Jacob C, Panchal S and Melotto M. Surface Inoculation and Quantification of Pseudomonas syringae Population in the Arabidopsis Leaf Apoplast. (2017). Bio Protoc. 7(5):e2167. doi: 10.21769/BioProtoc.2167. PMID: 28573169; PMCID: PMC5448416
⁶Stefanato F L, Trippel C, Uszkoreit S, Ferrafiat L, Grenga L, Dickens R, Kelly N, Kingdon ADH, Ambrosetti L, Findlay K C, Cheema J, Trick M, Chandra G, Tomalin G, Malone J G, Truman A W. (2019). Pan-genome analysis identifies intersecting roles for Pseudomonas specialized metabolites in potato pathogen inhibition. In review: bioRxiv 783258; doi: https://doi.org/10.1101/783258
⁷Hudson G A, and Mitchell D A. (2018). RiPP antibiotics: biosynthesis and engineering potential. Curr Opin Microbiol. 45:61-69. doi: 10.1016/j.mib.2018.02.010
⁸Reis M, Eschbach-Bludau M, Iglesias-Wind M I, Kupke T, Sahl H G. (1994). Producer immunity towards the lantibiotic Pep5: identification of the immunity gene pepI and localization and functional analysis of its gene product. Appl Environ Microbiol. 60(8):2876-2883. doi:10.1128/AEM.60.8.2876-2883.1994
⁹Folders J, Algra J, Roelofs M S, van Loon L C, Tommassen J, Bitter W. (2001). Characterization of Pseudomonas aeruginosa chitinase, a gradually secreted protein. J Bacteriol. 183(24):7044-7052. doi: 10.1128/J B.183.24.7044-7052.2001

TABLE 1
Example Target Cell Genera, Species & Strains
These may be useful, for example, where the target cell is comprised by
a plant (or any part of a plant disclosed herein), or an environment
(eg, a plant environment, eg, soil).

Acidovorax avenae subsp. cattleyae
Acidovorax avenae subsp.
Acidovorax konjaci
Acidovorax valerianellae
Agrobacterium
Agrobacterium larrymoorei
Agrobacterium radiobacter
Agrobacterium rhizogenes
Agrobacterium rubi
Agrobacterium tumefaciens
Agrobacterium vitis
Arthrobacter
Arthrobacter ilicis
Bacillus
Bacillus megaterium
Bacillus megaterium pv. cerealis
Bacillus pumilus
Brenneria
Brenneria alni
Brenneria nigrifluens
Brenneria quercina
Brenneria rubrifaciens
Brenneria salicis
Burkholderia
Burkholderia andropogonis
Burkholderia caryophylli
Burkholderia cepacia
Burkholderia gladioli
Burkholderia gladioli pv. agaricicola
Burkholderia gladioli pv. alliicola
Burkholderia gladioli pv. gladioli
Burkholderia glumae
Burkholderia
Clavibacter
Clavibacter michiganensis
Clavibacter michiganensis subsp.
Clavibacter michiganensis subsp. michiganensis
Clavibacter michiganensis subsp. nebraskensis
Clavibacter michiganensis subsp. tessellarius
Clavibacter michiganensis subsp. sepedonicus
Clavibacter michiganensis subsp. tessellarius
Clavibacter rathayi
Clavibacter toxicus
Clavibacter tritici
Clavibacter xyli
Clavibacter xyli subsp. cynodontis
Clavibacter xyli subsp. xyli
Clostridium
Clostridium puniceum
Corynebacterium
Corynebacterium betae
Corynebacterium beticola
Corynebacterium fascians
Corynebacterium flaccumfaciens
Corynebacterium flaccumfaciens pv. betae
Corynebacterium flaccumfaciens pv. flaccumfaciens
Corynebacterium flaccumfaciens pv. oortii
Corynebacterium flaccumfaciens pv. poinsettiae
Corynebacterium flaccumfaciens subsp.
Corynebacterium flaccumfaciens subsp. flaccumfaciens
Corynebacterium flaccumfaciens subsp. oortii
Corynebacterium flaccumfaciens subsp. poinsettiae
Corynebacterium ilicis
Corynebacterium insidiosum
Corynebacterium iranicum
Corynebacterium michiganense
Corynebacterium michiganensis pv. insidiosus
Corynebacterium michiganensis pv. iranicum
Corynebacterium michiganense pv. nebraskense
Corynebacterium michiganense pv. rathayi
Corynebacterium michiganense pv. sepedonicum
Corynebacterium michiganense pv. tritici
Corynebacterium michiganense subsp. insidiosum
Corynebacterium michiganense subsp.
Corynebacterium michiganense subsp. nebraskense
Corynebacterium michiganense subsp. sepedonicum
Corynebacterium michiganense subsp. tessellarius
Corynebacterium oortii
Corynebacterium
Corynebacterium rathayi
Corynebacterium sepedonicum
Corynebacterium tritici
Curtobacterium
Curtobacterium flaccumfaciens
Curtobacterium flaccumfaciens pv.
Curtobacterium flaccumfaciens pv. flaccumfaciens
Curtobacterium flaccumfaciens pv. ilicis
Curtobacterium flaccumfaciens pv. oortii
Curtobacterium flaccumfaciens pv. poinsettiae
Dickeya
Dickeya chrysanthemi
Dickeya chrysanthemi pv. chrysanthemi
Dickeya chrysanthemi pv. parthenii
Dickeya dadantii
Dickeya dianthicola
Dickeya dieffenbachiae
Dickeya paradisiaca
Dickeya zeae
Enterobacter
Enterobacter agglomerans
Enterobacter cancerogenus
Enterobacter cloacae
Enterobacter cloacae subsp. dissolvens
Enterobacter nimipressuralis
Enterobacter pyrinus
Erwinia
Erwinia alni
Erwinia amylovora.
Erwinia amylovora pv. pyri
Erwinia ananatis corrig.
Erwinia ananatis pv. ananatis
Erwinia ananas pv. uredovora
Erwinia cacticida
Erwinia cancerogena
Erwinia carnegieana
Erwinia carotovora
Erwinia carotovora pv. atroseptica
Erwinia carotovora pv. carotovora
Erwinia carotovora subsp. atroseptica
Erwinia carotovora subsp. carotovora
Erwinia carotovora subsp. betavasculorum
Erwinia carotovora subsp. odorifera
Erwinia carotovora subsp. wasabiae
Erwinia chrysanthemi
Erwinia chrysanthemi pv. chrysanthemi
Erwinia chrysanthemi pv.
Erwinia chrysanthemi pv. dieffenbachiae
Erwinia chrysanthemi pv. paradisiaca
Erwinia chrysanthemi pv. parthenii
Erwinia chrysanthemi pv. zeae
Erwinia cypripedii
Erwinia dissolvens
Erwinia herbicola
Erwinia herbicola f. sp.
Erwinia herbicola pv. millettiae
Erwinia mallotivora
Erwinia nigrifluens
Erwinia nimipressuralis
Erwinia papayae
Erwinia proteamaculans
Erwinia persicina
Enterobacter pyrinus
Erwinia psidii
Erwinia pyrifoliae
Erwinia rhapontici
Erwinia rubrifaciens
Erwinia salicis
Erwinia stewartii
Erwinia tracheiphila
Erwinia uredovora
Ewingella
Ewingella americana
Gluconobacter Asai
Gluconobacter oxydans
Herbaspirillum
Herbaspirillum rubrisubalbicans
Janthinobacterium
Janthinobacterium agaricidamnosum
Leifsonia
Leifsonia cynodontis
Leifsonia xyli
Leifsonia xyli subsp. cynodontis
Leifsonia xyli subsp. xyli
Nocardia
Nocardia vaccinii
Pantoea
Pantoea agglomerans
Pantoea agglomerans pv. gypsophilae
Pantoea agglomerans pv. millettiae
Pantoea ananatis
Pantoea ananatis pv. ananatis
Pantoea ananatis pv. uredovora
Pantoea stewartii
Pantoea stewartii subsp. indologenes
Pantoea stewartii subsp. stewartii
Pectobacterium
Pectobacterium
Pectobacterium
Pectobacterium cacticida corrig
Pectobacterium
Pectobacterium carotovorum
Pectobacterium carotovorum subsp. atrosepticum
Pectobacterium carotovorum subsp. betavasculorum
Pectobacterium carotovorum subsp. brasiliensis
Pectobacterium carotovorum subsp. carotovorum
Pectobacterium carotovorum subsp. odoriferum
Pectobacterium carotovorum subsp. wasabiae
Pectobacterium chrysanthemi
Pectobacterium chrysanthemi pv. chrysanthemi
Pectobacterium chrysanthemi pv. dianthicola
Pectobacterium chrysanthemi pv. dieffenbachiae
Pectobacterium chrysanthemi pv. parthenii
Pectobacterium chrysanthemi pv. zeae
Pectobacterium cypripedii
Pectobacterium rhapontici
Pectobacterium wasabiae
Pseudomonas
Pseudomonas agarici
Pseudomonas amygdali
Pseudomonas andropogonis pv. andropogonis
Pseudomonas andropogonis pv. sojae
Pseudomonas andropogonis pv. stizolobii
Pseudomonas asplenii
Pseudomonas avellanae
Pseudomonas avenae
Pseudomonas avenae subsp. avenae
Pseudomonas avenae subsp. citrulli
Pseudomonas avenae subsp. konjaci
Pseudomonas beteli corrig.
Pseudomonas cannabina
Pseudomonas caricapapayae
Pseudomonas caryophylli
Pseudomonas cattleyae
Pseudomonas cepacia
Pseudomonas cichorii
Pseudomonas cissicola
Pseudomonas coronafaciens
Pseudomonas corrugata
Pseudomonas costantinii
Pseudomonas dodoneae
Pseudomonas ficuserectae
Pseudomonas flectens
Pseudomonas fuscovaginae
Pseudomonas gingeri
Pseudomonas gladioli
Pseudomonas gladioli pv. agaricicola
Pseudomonas gladioli pv. alliicola
Pseudomonas gladioli pv. gladioli
Pseudomonas glumae
Pseudomonas hibiscicola
Pseudomonas marginalis
Pseudomonas marginalis pv. alfalfae
Pseudomonas marginalis pv. marginalis
Pseudomonas marginalis pv. pastinacae
Pseudomonas mediterranea
Pseudomonas meliae
Pseudomonas palleroniana
Pseudomonas plantarii
Pseudomonas pomi
Pseudomonas pseudoalcaligenes subsp. citrulli
Pseudomonas pseudoalcaligenes subsp. konjaci
Pseudomonas rubrilineans
Pseudomonas rubrisubalbicans
Pseudomonas salomonii
Pseudomonas savastanoi
Pseudomonas savastanoi pv. fraxini
Pseudomonas savastanoi pv. glycinea
Pseudomonas savastanoi pv. nerii
Pseudomonas savastanoi pv. phaseolicola
Pseudomonas savastanoi pv. retacarpa
Pseudomonas savastanoi pv. savastanoi
Pseudomonas syringae
Pseudomonas syringae pv. aceris
Pseudomonas syringae pv. actinidiae
Pseudomonas syringae pv. aesculi
Pseudomonas syringae pv. alisalensis
Pseudomonas syringae pv. antirrhini
Pseudomonas syringae pv. apii
Pseudomonas syringae pv. aptata
Pseudomonas syringae pv.
Pseudomonas syringae pv. atropurpurea
Pseudomonas syringae pv. avellanae
Pseudomonas syringae pv. avii
Pseudomonas syringae pv. berberidis
Pseudomonas syringae pv. broussonetiae
Pseudomonas syringae pv. castaneae
Pseudomonas syringae pv. cerasicola
Pseudomonas syringae pv. ciccaronei
Pseudomonas syringae pv. coriandricola
Pseudomonas syringae pv. coronafaciens
Pseudomonas syringae pv. coryli
Pseudomonas syringae pv. cunninghamiae
Pseudomonas syringae pv. daphniphylli
Pseudomonas syringae pv. delphinii
Pseudomonas syringae pv. dendropanacis
Pseudomonas syringae pv. dysoxyli
Pseudomonas syringae pv. eriobotryae
Pseudomonas syringae pv. garcae
Pseudomonas syringae pv. glycinea
Pseudomonas syringae pv. helianthi
Pseudomonas syringae pv.
Pseudomonas syringae pv.
Pseudomonas syringae pv.
Pseudomonas syringae pv. lapsa
Pseudomonas syringae pv. maculicola
Pseudomonas syringae pv.
Pseudomonas syringae pv. mori
Pseudomonas syringae pv. morsprunorum .
Pseudomonas syringae pv. myricae
Pseudomonas syringae pv.
Pseudomonas syringae pv. papulans
Pseudomonas syringae pv. passiflorae
Pseudomonas syringae pv.
Pseudomonas syringae pv. philadelphi
Pseudomonas syringae pv. photiniae
Pseudomonas syringae pv. pisi
Pseudomonas syringae pv. porri
Pseudomonas syringae pv. primulae
Pseudomonas syringae pv. rhaphiolepidis
Pseudomonas syringae pv. ribicola
Pseudomonas syringae pv. sesami
Pseudomonas syringae pv. solidagae
Pseudomonas syringae pv. spinaceae
Pseudomonas syringae pv. syringae
Pseudomonas syringae pv. tagetis
Pseudomonas syringae pv. theae
Pseudomonas syringae pv. tomato
Pseudomonas syringae pv. ulmi
Pseudomonas syringae pv. viburni
Pseudomonas syringae pv.
Pseudomonas syzygii
Pseudomonas tolaasii
Pseudomonas tremae
Pseudomonas viridiflava
Ralstonia
Ralstonia solanacearum
Ralstonia syzygii
Rathayibacter
Rathayibacter iranicus
Rathayibacter rathayi
Rathayibacter
Rathayibacter tritici
Rhizobacter
Rhizobacter dauci corrig.
Rhizobium
Rhizobium larrymoorei
Rhizobium radiobacter
Rhizobium rhizogenes
Rhizobium rubi
Rhizobium vitis
Rhodococcus
Rhodococcus fascians
Samsonia
Samsonia erythrinae
Serratia
Serratia marcescens
Serratia proteamaculans
Sphingomonas
Sphingomonas melonis Buonaurio
Sphingomonas suberifaciens
Spiroplasma
Spiroplasma citri
Spiroplasma kunkelii
Spiroplasma phoeniceum
Streptomyces
Streptomyces acidiscabies
Streptomyces albidoflavus
Streptomyces candidus
Streptomyces caviscabies
Streptomyces collinus
Streptomyces europaeiscabiei
Streptomyces intermedius
Streptomyces ipomoeae
Streptomyces luridiscabiei
Streptomyces niveiscabiei
Streptomyces puniciscabiei
Streptomyces reticuliscabei
Streptomyces scabiei corrig.
Streptomyces setonii
Streptomyces steliiscabiei
Streptomyces turgidiscabies
Streptomyces wedmorensis
Xanthomonas
Xanthomonas albilineans
Xanthomonas alfalfae
Xanthomonas alfalfae subsp. alfalfae
Xanthomonas alfalfae subsp. citrumelonis
Xanthomonas arboricola
Xanthomonas axonopodis
Xanthomonas bromi
Xanthomonas campestris
Xanthomonas cassava
Xanthomonas citri
Xanthomonas cucurbitae
Xanthomonas euvesicatoria
Xanthomonas fragariae
Xanthomonas fuscans
Xanthomonas fuscans
Xanthomonas gardneri
Xanthomonas hortorum
Xanthomonas hortorum
Xanthomonas hyacinthi
Xanthomonas oryzae
Xanthomonas populi
Xanthomonas sacchari
Xanthomonas theicola
Xanthomonas translucens
Xanthomonas vasicola
Xylella
Xylella fastidiosa
Xylophilus
Xylophilus ampelinus
‘Candidatus' Plant Pathogenic Bacteria
’Candidatus Liberibacter’
‘Candidatus Liberibacter asiaticus'
‘Candidatus Phlomobacter’
‘Candidatus Phlomobacter fragariae’
‘Candidatus Phytoplasma’

TABLE 2
Further Example Bacteria
Optionally, the carrier cells are selected from this Table and/or the target cells are selected from this Table (eg, wherein the carrier and target cells are of a different species; or of the same species but are a different strain or the carrier cells are engineered but the target cells are wild-type or vice versa). For example the carrier cells are E coli cells and the target cells are C dificile, E coli, Akkermansia, Enterobacteriacea, Ruminococcus, Faecalibacterium, Firmicutes, Bacteroidetes, Salmonella, Klebsiella, Pseudomonas, Acintenobacter or Streptococcus cells.

| | | | | |
|---|---|---|---|---|
| Abiotrophia | Acidocella | Actinomyces | Alkalilimnicola | Aquaspirillum |
| Abiotrophia defectiva | Acidocella aminolytica | Actinomyces bovis | Alkalilimnicola ehrlichii | Aquaspirillum polymorphum |
| Acaricomes | Acidocella facilis | Actinomyces denticolens | Alkaliphilus | Aquaspirillum putridiconchylium |
| Acaricomes phytoseiuli | Acidomonas | Actinomyces europaeus | Alkaliphilus oremlandii | Aquaspirillum serpens |
| Acetitomaculum | Acidomonas methanolica | Actinomyces georgiae | Alkaliphilus transvaalensis | Aquimarina |
| Acetitomaculum ruminis | Acidothermus | Actinomyces gerencseriae | Allochromatium | Aquimarina latercula |
| Acetivibrio | Acidothermus cellulolyticus | Actinomyces hordeovulneris | Allochromatium vinosum | Arcanobacterium |
| Acetivibrio cellulolyticus | Acidovorax | Actinomyces howellii | Alloiococcus | Arcanobacterium haemolyticum |
| Acetivibrio ethanolgignens | Acidovorax anthurii | Actinomyces hyovaginalis | Alloiococcus otitis | Arcanobacterium pyogenes |
| Acetivibrio multivorans | Acidovorax caeni | Actinomyces israelii | Allokutzneria | Archangium |
| Acetoanaerobium | Acidovorax cattleyae | Actinomyces johnsonii | Allokutzneria albata | Archangium gephyra |
| Acetoanaerobium noterae | Acidovorax citrulli | Actinomyces meyeri | Altererythrobacter | Arcobacter |
| Acetobacter | Acidovorax defluvii | Actinomyces naeslundii | Altererythrobacter ishigakiensis | Arcobacter butzleri |
| Acetobacter aceti | Acidovorax delafieldii | Actinomyces neuii | Altermonas | Arcobacter cryaerophilus |
| Acetobacter cerevisiae | Acidovorax facilis | Actinomyces odontolyticus | Altermonas haloplanktis | Arcobacter halophilus |
| Acetobacter cibinongensis | Acidovorax konjaci | Actinomyces oris | Altermonas macleodii | Arcobacter nitrofigilis |
| Acetobacter estunensis | Acidovorax temperans | Actinomyces radingae | Alysiella | Arcobacter skirrowii |
| Acetobacter fabarum | Acidovorax valerianellae | Actinomyces slackii | Alysiella crassa | Arhodomonas |
| Acetobacter ghanensis | Acinetobacter | Actinomyces turicensis | Alysiella filiformis | Arhodomonas aquaeolei |
| Acetobacter indonesiensis | Acinetobacter baumannii | Actinomyces viscosus | Aminobacter | Arsenophonus |
| Acetobacter lovaniensis | Acinetobacter baylyi | Actinoplanes | Aminobacter aganoensis | Arsenophonus nasoniae |
| Acetobacter malorum | Acinetobacter bouvetii | Actinoplanes auranticolor | Aminobacter aminovorans | Arthrobacter |
| Acetobacter nitrogenifigens | Acinetobacter calcoaceticus | Actinoplanes brasiliensis | Aminobacter niigataensis | Arthrobacter agilis |
| Acetobacter oeni | Acinetobacter gerneri | Actinoplanes consettensis | Aminobacterium | Arthrobacter albus |
| Acetobacter orientalis | Acinetobacter haemolyticus | Actinoplanes deccanensis | Aminobacterium mobile | Arthrobacter aurescens |
| Acetobacter orleanensis | Acinetobacter johnsonii | Actinoplanes derwentensis | Aminomonas | Arthrobacter chlorophenolicus |
| Acetobacter pasteurianus | Acinetobacter junii | Actinoplanes digitatis | Aminomonas paucivorans | Arthrobacter citreus |
| Acetobacter pornorurn | Acinetobacter lwoffi | Actinoplanes durhamensis | Ammoniphilus | Arthrobacter crystallopoietes |
| Acetobacter senegalensis | Acinetobacter parvus | Actinoplanes ferrugineus | Ammoniphilus oxalaticus | Arthrobacter cumminsii |
| Acetobacter xylinus | Acinetobacter radioresistens | Actinoplanes globisporus | Ammoniphilus oxalivorans | Arthrobacter globiformis |
| Acetobacterium | Acinetobacter schindleri | Actinoplanes humidus | Amphibacillus | Arthrobacter histidinolovorans |
| Acetobacterium bakii | Acinetobacter soli | Actinoplanes italicus | Amphibacillus xylanus | Arthrobacter ilicis |
| Acetobacterium carbinolicum | Acinetobacter tandoii | Actinoplanes liguriensis | Amphritea | Arthrobacter luteus |
| Acetobacterium dehalogenans | Acinetobacter tjernbergiae | Actinoplanes lobatus | Amphritea balenae | Arthrobacter methylotrophus |
| Acetobacterium fimetarium | Acinetobacter towneri | Actinoplanes missouriensis | Amphritea japonica | Arthrobacter mysorens |
| Acetobacterium malicum | Acinetobacter ursingii | Actinoplanes palleronii | Amycolatopsis | Arthrobacter nicotianae |
| Acetobacterium paludosum | Acinetobacter venetianus | Actinoplanes philippinensis | Amycolatopsis alba | Arthrobacter nicotinovorans |
| Acetobacterium tundrae | Acrocarpospora | Actinoplanes rectilineatus | Amycolatopsis albidoflavus | Arthrobacter oxydans |
| Acetobacterium wieringae | Acrocarpospora corrugata | Actinoplanes regularis | Amycolatopsis azurea | Arthrobacter pascens |
| Acetobacterium woodii | Acrocarpospora macrocephala | Actinoplanes teichomyceticus | Amycolatopsis coloradensis | Arthrobacter phenanthrenivorans |
| Acetofilamentum | Acrocarpospora pleiomorpha | Actinoplanes utahensis | Amycolatopsis lurida | Arthrobacter polychromogenes |
| Acetofilamentum rigidum | Actibacter | Actinopolyspora | Amycolatopsis mediterranei | Atrhrobacter protophormiae |
| Acetohalobium | Actibacter sediminis | Actinopolyspora halophila | Amycolatopsis rifamycinica | Arthrobacter psychrolactophilus |
| Acetohalobium arabaticum | Actinoalloteichus | Actinopolyspora mortivallis | Amycolatopsis rubida | Arthrobacter ramosus |
| Acetomicrobium | Actinoalloteichus cyanogriseus | Actinosynnema | Amycolatopsis sulphurea | Arthrobacter sulfonivorans |
| Acetomicrobium faecale | Actinoalloteichus hymeniacidonis | Actinosynnema mirum | Amycolatopsis tolypomycina | Arthrobacter sulfureus |
| Acetomicrobium flavidum | Actinoalloteichus spitiensis | Actinotalea | Anabaena | Arthrobacter uratoxydans |
| Acetonema | Actinobaccillus | Actinotalea fermentans | Anabaena cylindrica | Arthrobacter ureafaciens |
| Acetonema longum | Actinobacillus capsulatus | Aerococcus | Anabaena flos-aquae | Arthrobacter viscosus |
| Acetothermus | Actinobacillus delphinicola | Aerococcus sanguinicola | Anabaena variabilis | Arthrobacter woluwensis |
| Acetothermus paucivorans | Actinobacillus hominis | Aerococcus urinae | Anaeroarcus | Asaia |
| Acholeplasma | Actinobacillus indolicus | Aerococcus urinaeequi | Anaeroarcus burkinensis | Asaia bogorensis |
| Acholeplasma axanthum | Actinobacillus lignieresii | Aerococcus urinaehominis | Anaerobaculum | Asanoa |
| Acholeplasma brassicae | Actinobacillus minor | Aerococcus viridans | Anaerobaculum mobile | Asanoa ferruginea |
| Acholeplasma cavigenitalium | Actinobacillus muris | Aeromicrobium | Anaerobiospirillum | Asticcacaulis |
| Acholeplasma equifetale | Actinobacillus pleuropneumoniae | Aeromicrobium erythreum | Anaerobiospirillum succiniciproducens | Asticcacaulis biprosthecium |
| Acholeplasma granularum | Actinobacillus porcinus | Aeromonas | Anaerobiospirillum thomasii | Asticcacaulis excentricus |
| Acholeplasma hippikon | Actinobacillus rossii | Aeromonas allosaccharophila | Anaerococcus | Atopobacter |
| Acholeplasma laidlawii | Actinobacillus scotiae | Aeromonas bestiarum | Anaerococcus hydrogenalis | Atopobacter phocae |
| Acholeplasma modicum | Actinobacillus seminis | Aeromonas caviae | Anaerococcus lactolyticus | Atopobium |
| Acholeplasma morum | Actinobacillus succinogenes | Aeromonas encheleia | Anaerococcus prevotii | Atopobium fossor |
| Acholeplasma multilocale | Actinobaccillus suis | Aeromonas enteropelogenes | Anaerococcus tetradius | Atopobium minutum |
| Acholeplasma oculi | Actinobacillus ureae | Aeromonas eucrenophila | Anaerococcus vaginalis | Atopobium parvulum |
| Acholeplasma palmae | Actinobaculum | Aeromonas ichthiosmia | Anaerofustis | Atopobium rimae |
| Acholeplasma parvum | Actinobaculum massiliense | Aeromonas jandaei | Anaerofustis stercorihominis | Atopobium vaginae |
| Acholeplasma pleciae | Actinobaculum schaalii | Aeromonas media | Anaeromusa | Aureobacterium |
| Acholeplasma vituli | Actinobaculum suis | Aeromonas popoffii | Anaeromusa acidaminophila | Aureobacterium barkeri |
| Achromobacter | Actinomyces urinale | Aeromonas sobria | Anaeromyxobacter | Aurobacterium |
| Achromobacter denitrificans | Actinocatenispora | Aeromonas veronii | Anaeromyxobacter dehalogenans | Aurobacterium liquefaciens |
| Achromobacter insolitus | Actinocatenispora rupis | Agrobacterium | Anaerorhabdus | Avibacterium |
| Achromobacter piechaudii | Actinocatenispora thailandica | Agrobacterium gelatinovorum | Anaerorhabdus furcosa | Avibacterium avium |
| Achromobacter ruhlandii | Actinocatenispora sera | Agrococcus | Anaerosinus | Avibacterium gallinarum |
| Achromobacter spanius | Actinocorallia | Agrococcus citreus | Anaerosinus glycerini | Avibacterium paragallinarum |
| Acidaminobacter | Actinocorallia aurantiaca | Agrococcus jenensis | Anaerovirgula | Avibacterium volantium |
| Acidaminobacter | Actinocorallia aurea | Agromonas | Anaerovirgula multivorans | Azoarcus |
| hydrogenoformans | Actinocorallia cavernae | Agromonas oligotrophica | Ancalomicrobium | Azoarcus indigens |
| Acidaminococcus | Actinocorallia glomerata | Agromyces | Ancalomicrobium adetum | Azoarcus tolulyticus |
| Acidaminococcus fermentans | Actinocorallia herbida | Agromyces fucosus | Ancylobacter | Azoarcus toluvorans |
| Acidaminococcus intestini | Actinocorallia libanotica | Agromyces hippuratus | Ancylobacter aquaticus | Azohydromonas |
| Acidicaldus | Actinocorallia longicatena | Agromyces luteolus | Aneurinibacillus | Azohydromonas australica |
| Acidicaldus organivorans | Actinomadura | Agromyces mediolanus | Aneurinibacillus aneurinilyticus | Azohydromonas lata |
| Acidimicrobium | Actinomadura alba | Agromyces ramosus | Aneurinibacillus migulanus | Azomonas |
| Acidimicrobium ferrooxidans | Actinomadura atramentaria | Agromyces rhizospherae | Aneurinibacillus thermoaerophilus | Azomonas agilis |
| Acidiphilium | Actinomadura bangladeshensis | Akkermansia | Angiococcus | Azomonas insignis |
| Acidiphilium acidophilum | Actinomadura catellatispora | Akkermansia muciniphila | Angiococcus disciformis | Azomonas macrocytogenes |
| Acidiphilium angustum | Actinomadura chibensis | Albidiferax | Angulomicrobium | Azorhizobium |
| Acidiphilium cryptum | Actinomadura chokoriensis | Albidiferax ferrireducens | Angulomicrobium tetraedrale | Azorhizobium caulinodans |
| Acidiphilium multivorum | Actinomadura citrea | Albidovulum | Anoxybacillus | Azorhizophilus |
| Acidiphilium organovorum | Actinomadura coerulea | Albidovulum inexpectatum | Anoxybacillus pushchinoensis | Azorhizophilus paspali |
| Acidiphilium rubrum | Actinomadura echinospora | Alcaligenes | Aquabacterium | Azospirillum |
| Acidisoma | Actinomadura fibrosa | Alcaligenes denitrificans | Aquabacterium commune | Azospirillum brasilense |
| Acidisoma sibiricum | Actinomadura formosensis | Alcaligenes faecalis | Aquabacterium parvum | Azospirillum halopraeferens |
| Acidisoma tundrae | Actinomadura hibisca | Alcanivorax | Borrelia | Azospirillum irakense |
| Acidisphaera | Actinomadura kijaniata | Alcanivorax borkumensis | Borrelia afzelii | Azotobacter |
| Acidisphaera rubrifaciens | Actinomadura latina | Alcanivorax jadensis | Borrelia americana | Azotobacter beijerinckii |
| Acidithiobacillus | Actinomadura livida | Algicola | Borrelia burgdorferi | Azotobacter chroococcum |
| Acidithiobacillus albertensis | Actinomadura luteofluorescens | Algicola bacteriolytica | Borrelia carolinensis | Azotobacter nigricans |
| Acidithiobacillus caldus | Actinomadura macra | Alicyclobacillus | Borrelia coriaceae | Azotobacter salinestris |
| Acidithiobacillus ferrooxidans | Actinomadura madurae | Alicyclobacillus disulfidooxidans | Borrelia garinii | Azotobacter vinelandii |
| Acidithiobacillus thiooxidans | Actinomadura oligospora | Alicyclobacillus sendaiensis | Borrelia japonica | Brevinema |
| Acidobacterium | Actinomadura pelletieri | Alicyclobacillus vulcanalis | Bosea | Brevinema andersonii |
| Acidobacterium capsulatum | Actinomadura rubrobrunea | Alishewanella | Bosea minatitlanensis | Brevundimonas |
| Bacillus | Actinomadura rugatobispora | Alishewanella fetalis | Bosea thiooxidans | Brevundimonas alba |
| [see below] | Actinomadura umbrina | Alkalibacillus | Brachybacterium | Brevundimonas aurantiaca |
| Bacteriovorax | Actinomadura verrucosospora | Alkalibacillus haloalkaliphilus | Brachybacterium alimentarium | Brevundimonas diminuta |
| Bacteriovorax stolpii | Actinomadura vinacea | Bibersteinia | Brachybacterium faecium | Brevundimonas intermedia |
| B. acidiceler | Actinomadura viridilutea | Bibersteinia trehalosi | Brachybacterium paraconglomeratum | Brevundimonas subvibrioides |
| B. acidicola | Actinomadura viridis | Bifidobacterium | Brachybacterium rhamnosum | Brevundimonas vancanneytii |
| B. acidiproducens | Actinomadura yumaensis | Bifidobacterium adolescentis | Brachybacterium tyrofermentans | Brevundimonas variabilis |
| B. acidocaldarius | Bacteroides | Bifidobacterium angulatum | Brachyspira | Brevundimonas vesicularis |
| B. acidoterrestris | Bacteroides caccae | Bifidobacterium animalis | Brachyspira alvinipulli | Brochothrix |
| B. aeolius | Bacteroides coagulans | Bifidobacterium asteroides | Brachyspira hyodysenteriae | Brochothrix campestris |
| B. aerius | Bacteroides eggerthii | Bifidobacterium bifidum | Brachyspira innocens | Brochothrix thermosphacta |
| B. aerophilus | Bacteroides fragilis | Bifidobacterium boum | Brachyspira murdochii | Brucella |
| B. agaradhaerens | Bacteroides galacturonicus | Bifidobacterium breve | Brachyspira pilosicoli | Brucella canis |
| B. agri | Bacteroides helcogenes | Bifidobacterium catenulatum | Bradyrhizobium | Brucella neotomae |
| B. aidingensis | Bacteroides ovatus | Bifidobacterium choerinum | Bradyrhizobium canariense | Bryobacter |
| B. akibai | Bacteroides pectinophilus | Bifidobacterium coryneforme | Bradyrhizobium elkanii | Bryobacter aggregatus |
| B. alcalophilus | Bacteroides pyogenes | Bifidobacterium cuniculi | Bradyrhizobium japonicum | Burkholderia |
| B. algicola | Bacteroides salyersiae | Bifidobacterium dentium | Bradyrhizobium liaoningense | Burkholderia ambifaria |
| B. alginolyticus | Bacteroides stercoris | Bifidobacterium gallicum | Brenneria | Burkholderia andropogonis |
| B. alkalidiazotrophicus | Bacteroides suis | Bifidobacterium gallinarum | Brenneria alni | Burkholderia anthina |
| B. alkalinitrilicus | Bacteroides tectus | Bifidobacterium indicum | Brenneria nigrifluens | Burkholderia caledonica |
| B. alkalisediminis | Bacteroides thetaiotaomicron | Bifidobacterium longum | Brenneria quercina | Burkholderia caryophylli |
| B. alkalitelluris | Bacteroides uniformis | Bifidobacterium | Brenneria quercina | Burkholderia cenocepacia |
| B. altitudinis | Bacteroides ureolyticus | magnumBifidobacterium | Brenneria salicis | Burkholderia cepacia |
| B. alveayuensis | Bacteroides vulgatus | merycicum | Brevibacillus | Burkholderia cocovenenans |
| B. alvei | Balnearium | Bifidobacterium minimum | Brevibacillus agri | Burkholderia dolosa |
| B. amyloliquefaciens | Balnearium lithotrophicum | Bifidobacterium pseudocatenulatum | Brevibacillus borstelensis | Burkholderia fungorum |
| B. a. subsp. amyloliquefaciens | Balneatrix | Bifidobacterium pseudolongum | Brevibacillus brevis | Burkholderia glathei |
| B. a. subsp. plantarum | Balneatrix alpica | Bifidobacterium pullorum | Brevibacillus centrosporus | Burkholderia glumae |
| B. dipsosauri | Balneola | Bifidobacterium ruminantium | Brevibacillus choshinensis | Burkholderia graminis |
| B. drentensis | Balneola vulgaris | Bifidobacterium saeculare | Brevibacillus invocatus | Burkholderia kururiensis |
| B. edaphicus | Barnesiella | Bifidobacterium subtile | Brevibacillus laterosporus | Burkholderia multivorans |
| B. ehimensis | Barnesiella viscericola | Bifidobacterium thermophilum | Brevibacillus parabrevis | Burkholderia phenazinium |
| B. eiseniae | Bartonella | Bilophila | Brevibacillus reuszeri | Burkholderia plantarii |
| B. enclensis | Bartonella alsatica | Bilophila wadsworthia | Brevibacterium | Burkholderia pyrrocinia |
| B. endophyticus | Bartonella bacilliformis | Biostraticola | Brevibacterium abidum | Burkholderia silvatlantica |
| B. endoradicis | Bartonella clarridgeiae | Biostraticola tofi | Brevibacterium album | Burkholderia stabilis |
| B. farraginis | Bartonella doshiae | Bizionia | Brevibacterium aurantiacum | Burkholderia thailandensis |
| B. fastidiosus | Bartonella elizabethae | Bizionia argentinensis | Brevibacterium celere | Burkholderia tropica |
| B. fengqiuensis | Bartonella grahamii | Blastobacter | Brevibacterium epidermidis | Burkholderia unamae |
| B. firmus | Bartonella henselae | Blastobacter capsulatus | Brevibacterium frigoritolerans | Burkholderia vietnamiensis |
| B. flexus | Bartonella rochalimae | Blastobacter denitrificans | Brevibacterium halotolerans | Buttiauxella |
| B. foraminis | Bartonella vinsonii | Blastococcus | Brevibacterium iodinum | Buttiauxella agrestis |
| B. fordii | Bavariicoccus | Blastococcus aggregatus | Brevibacterium linens | Buttiauxella brennerae |
| B. formosus | Bavariicoccus seileri | Blastococcus saxobsidens | Brevibacterium lyticum | Buttiauxella ferragutiae |
| B. fortis | Bdellovibrio | Blastochloris | Brevibacterium mcbrellneri | Buttiauxella gaviniae |
| B. fumarioli | Bdellovibrio bacteriovorus | Blastochloris viridis | Brevibacterium otitidis | Buttiauxella izardii |
| B. funiculus | Bdellovibrio exovorus | Blastomonas | Brevibacterium oxydans | Buttiauxella noackiae |
| B. fusiformis | Beggiatoa | Blastomonas natatoria | Brevibacterium paucivorans | Buttiauxella warmboldiae |
| B. galactophilus | Beggiatoa alba | Blastopirellula | Brevibacterium stationis | Butyrivibrio |
| B. galactosidilyticus | Beijerinckia | Blastopirellula marina | B. taeanensis | Butyrivibrio fibrisolvens |
| B. galliciensis | Beijerinckia derxii | Blautia | B. tequilensis | Butyrivibrio hungatei |
| B. gelatini | Beijerinckia fluminensis | Blautia coccoides | B. thermantarcticus | Butyrivibrio proteoclasticus |
| B. gibsonii | Beijerinckia indica | Blautia hansenii | B. thermoaerophilus | B. lautus |
| B. ginsengi | Beijerinckia mobilis | Blautia producta | B. thermoamylovorans | B. lehensis |
| B. ginsengihumi | Belliella | Blautia wexlerae | B. thermocatenulatus | B. lentimorbus |
| B. ginsengisoli | Belliella baltica | Bogoriella | B. thermocloacae | B. lentus |
| B. globisporus (eg, B. | Bellilinea | Bogoriella caseilytica | B. thermocopriae | B. licheniformis |
| g. subsp. Globisporus; or B. | Bellilinea caldifistulae | Bordetella | B. thermodenitrificans | B. ligniniphilus |
| g. subsp. Marinus) | Belnapia | Bordetella avium | B. thermoglucosidasius | B. litoralis |
| Caenimonas | Belnapia moabensis | Bordetella bronchiseptica | B. thermolactis | B. locisalis |
| Caenimonas koreensis | Bergeriella | Bordetella hinzii | B. thermoleovorans | B. luciferensis |
| Caldalkalibacillus | Bergeriella denitrificans | Bordetella holmesii | B. thermophilus | B. luteolus |
| Caldalkalibacillus uzonensis | Beutenbergia | Bordetella parapertussis | B. thermoruber | B. luteus |
| Caldanaerobacter | Beutenbergia cavernae | Bordetella pertussis | B. thermosphaericus | B. macauensis |
| Caldanaerobacter subterraneus | B. aminovorans | Bordetella trematum | B. thiaminolyticus | B. macerans |
| Caldanaerobius | B. amylolyticus | B. gordonae | B. thioparans | B. macquariensis |
| Caldanaerobius fijiensis | B. andreesenii | B. gottheilii | B. thuringiensis | B. macyae |
| Caldanaerobius polysaccharolyticus | B. aneurinilyticus | B. graminis | B. tianshenii | B. malacitensis |
| Caldanaerobius zeae | B. anthracis | B. halmapalus | B. trypoxylicola | B. mannanilyticus |
| Caldanaerovirga | B. aquimaris | B. haloalkaliphilus | B. tusciae | B. marisflavi |
| Caldanaerovirga acetigignens | B. arenosi | B. halochares | B. validus | B. marismortui |
| Caldicellulosiruptor | B. arseniciselenatis | B. halodenitrificans | B. vallismortis | B. marmarensis |
| Caldicellulosiruptor bescii | B. arsenicus | B. halodurans | B. vedderi | B. massiliensis |
| Caldicellulosiruptor kristjanssonii | B. aurantiacus | B. halophilus | B. velezensis | B. megaterium |
| Caldicellulosiruptor owensensis | B. arvi | B. halosaccharovorans | B. vietnamensis | B. mesonae |
| | B. aryabhattai | B. hemicellulosilyticus | B. vireti | B. methanolicus |
| | B. asahii | B. hemicentroti | B. vulcani | B. methylotrophicus |
| | B. atrophaeus | B. herbersteinensis | B. wakoensis | B. migulanus |
| | B. axarquiensis | B. horikoshii | B. weihenstephanensis | B. mojavensis |
| | B. azotofixans | B. horneckiae | B. xiamenensis | B. mucilaginosus |
| | B. azotoformans | B. horti | B. xiaoxiensis | B. muralis |
| | B. badius | B. huizhouensis | B. zhanjiangensis | B. murimartini |
| | B. barbaricus | B. humi | B. peoriae | B. mycoides |
| | B. bataviensis | B. hwajinpoensis | B. persepolensis | B. naganoensis |
| | B. beijingensis | B. idriensis | B. persicus | B. nanhaiensis |
| | B. benzoevorans | B. indicus | B. pervagus | B. nanhaiisediminis |
| | B. beringensis | B. infantis | B. plakortidis | B. nealsonii |
| | B. berkeleyi | B. infernus | B. pocheonensis | B. neidei |
| | B. beveridgei | B. insolitus | B. polygoni | B. neizhouensis |
| | B. bogoriensis | B. invictae | B. polymyxa | B. niabensis |
| | B. boroniphilus | B. iranensis | B. popilliae | B. niacini |
| | B. borstelensis | B. isabeliae | B. pseudalcalophilus | B. novalis |
| | B. brevis Migula | B. isronensis | B. pseudofirmus | B. oceanisediminis |
| | B. butanolivorans | B. jeotgali | B. pseudomycoides | B. odysseyi |
| | B. canaveralius | B. kaustophilus | B. psychrodurans | B. okhensis |
| | B. carboniphilus | B. kobensis | B. psychrophilus | B. okuhidensis |
| | B. cecembensis | B. kochii | B. psychrosaccharolyticus | B. oleronius |
| | B. cellulosilyticus | B. kokeshiiformis | B. psychrotolerans | B. oryzaecorticis |
| | B. centrosporus | B. koreensis | B. pulvifaciens | B. oshimensis |
| | B. cereus | B. korlensis | B. pumilus | B. pabuli |
| | B. chagannorensis | B. kribbensis | B. purgationiresistens | B. pakistanensis |
| | B. chitinolyticus | B. krulwichiae | B. pycnus | B. pallidus |
| | B. chondroitinus | B. laevolacticus | B. qingdaonensis | B. pallidus |
| | B. choshinensis | B. larvae | B. qingshengii | B. panacisoli |
| | B. chungangensis | B. laterosporus | B. reuszeri | B. panaciterrae |
| | B. cibi | B. salexigens | B. rhizosphaerae | B. pantothenticus |
| | B. circulans | B. saliphilus | B. rigui | B. parabrevis |
| | B. clarkii | B. schlegelii | B. ruris | B. paraflexus |
| | B. clausii | B. sediminis | B. safensis | B. pasteurii |
| | B. coagulans | B. selenatarsenatis | B. salarius | B. patagoniensis |
| | B. coahuilensis | B. selenitireducens | Catenuloplanes | Curtobacterium |
| | B. cohnii | B. seohaeanensis | Catenuloplanes atrovinosus | Curtobacterium albidum |
| | B. composti | B. shacheensis | Catenuloplanes castaneus | Curtobacterium citreus |
| | B. curdlanolyticus | B. shackletonii | Catenuloplanes crispus | |
| | B. cycloheptanicus | B. siamensis | Catenuloplanes indicus | |
| | B. cytotoxicus | B. silvestris | Catenuloplanes japonicus | |
| | B. daliensis | B. simplex | Catenuloplanes nepalensis | |
| | B. decisifrondis | B. siralis | Catenuloplanes niger | |
| | B. decolorationis | B. smithii | Chryseobacterium | |
| | B. deserti | B. soli | Chryseobacterium balustinum | |
| | Campylobacter | B. solimangrovi | Citrobacter | |
| | Campylobacter coli | B. solisalsi | C. amalonaticus | |
| | Campylobacter concisus | B. songklensis | C. braakii | |
| | Campylobacter curvus | B. sonorensis | C. diversus | |
| | Campylobacter fetus | B. sphaericus | C. farmeri | |
| | Campylobacter gracilis | B. sporothermodurans | C. freundii | |
| | Campylobacter helveticus | B. stearothermophilus | C. gillenii | |
| | Campylobacter hominis | B. stratosphericus | C. koseri | |
| | Campylobacter hyointestinalis | B. subterraneus | C. murliniae | |
| | Campylobacter jejuni | B. subtilis (eg, B. | C. pasteurii^{[1]} | |
| | Campylobacter lari | s. subsp. Inaquosorum; or B. | C. rodentium | |
| | Campylobacter mucosalis | s. subsp. Spizizeni; or B. | C. sedlakii | |
| | Campylobacter rectus | s. subsp. Subtilis) | C. werkmanii | |
| | Campylobacter showae | Cardiobacterium | C. youngae | |
| | Campylobacter sputorum | Cardiobacterium hominis | Clostridium | |
| | Campylobacter upsaliensis | Carnimonas | (see below) | |
| | Capnocytophaga | Carnimonas nigrificans | Coccochloris | |
| | Capnocytophaga canimorsus | Carnobacterium | Coccochloris elabens | |
| | Capnocytophaga cynodegmi | Carnobacterium alterfunditum | Corynebacterium | |
| | Capnocytophaga gingivalis | Carnobacterium divergens | Corynebacterium flavescens | |
| | Capnocytophaga granulosa | Carnobacterium funditum | Corynebacterium variabile | |
| | Capnocytophaga haemolytica | Carnobacterium gallinarum | | |
| | Capnocytophaga ochracea | Carnobacterium maltaromaticum | | |
| | Capnocytophaga sputigena | Carnobacterium mobile | | |
| | | Carnobacterium viridans | | |
| | | Caryophanon | | |
| | | Caryophanon latum | | |
| | | Caryophanon tenue | | |
| | | Catellatospora | | |
| | | Catellatospora citrea | | |
| | | Catellatospora methionotrophica | | |
| | | Catenococcus | | |
| | | Catenococcus thiocycli | | |
| Clostridium | | | | |

Clostridium absonum, Clostridium aceticum, Clostridium acetireducens, Clostridium acetobutylicum, Clostridium acidisoli, Clostridium aciditolerans,
Clostridium acidurici, Clostridium aerotolerans, Clostridium aestuarii, Clostridium akagii, Clostridium aldenense, Clostridium aldrichii, Clostridium
algidicarni, Clostridium algidixylanolyticum, Clostridium algifaecis, Clostridium algoriphilum, Clostridium alkalicellulosi, Clostridium aminophilum,
Clostridium aminovalericum, Clostridium amygdalinum, Clostridium amylolyticum, Clostridium arbusti, Clostridium arcticum, Clostridium argentinense,
Clostridium asparagiforme, Clostridium aurantibutyricum, Clostridium autoethanogenum, Clostridium baratii, Clostridium barkeri, Clostridium bartlettii,
Clostridium beijerinckii, Clostridium bifermentans, Clostridium bolteae, Clostridium bornimense, Clostridium botulinum, Clostridium bowmanii, Clostridium
bryantii, Clostridium butyricum, Clostridium cadaveris, Clostridium caenicola, Clostridium caminithermale, Clostridium carboxidivorans, Clostridium carnis,
Clostridium cavendishii, Clostridium celatum, Clostridium celerecrescens, Clostridium cellobioparum, Clostridium cellulofermentans, Clostridium
cellulolyticum, Clostridium cellulosi, Clostridium cellulovorans, Clostridium chartatabidum, Clostridium chauvoei, Clostridium chromiireducens, Clostridium
citroniae, Clostridium clariflavum, Clostridium clostridioforme, Clostridium coccoides, Clostridium cochlearium, Clostridium colletant, Clostridium colicanis,
Clostridium colinum, Clostridium collagenovorans, Clostridium cylindrosporum, Clostridium difficile, Clostridium diolis, Clostridium disporicum,
Clostridium drakei, Clostridium durum, Clostridium estertheticum, Clostridium estertheticum estertheticum, Clostridium estertheticum laramiense,
Clostridium fallax, Clostridium felsineum, Clostridium fervidum, Clostridium fimetarium, Clostridium formicaceticum, Clostridium frigidicarnis,Clostridium
frigoris, Clostridium ganghwense, Clostridium gasigenes, Clostridium ghonii, Clostridium glycolicum, Clostridium glycyrrhizinilyticum, Clostridium grantii,
Clostridium haemolyticum, Clostridium halophilum, Clostridium hastiforme, Clostridium hathewayi, Clostridium herbivorans, Clostridium hiranonis,
Clostridium histolyticum, Clostridium homopropionicum, Clostridium huakuii, Clostridium hungatei, Clostridium hydrogeniformans, Clostridium
hydroxybenzoicum, Clostridium hylemonae, Clostridium jejuense, Clostridium indolis, Clostridium innocuum, Clostridium intestinale, Clostridium irregulare,
Clostridium isatidis, Clostridium josui, Clostridium kluyveri, Clostridium lactatifermentans, Clostridium lacusfryxellense, Clostridium laramiense, Clostridium
lavalense, Clostridium lentocellum, Clostridium lentoputrescens, Clostridium leptum, Clostridium limosum, Clostridium litorale, Clostridium lituseburense,
Clostridium ljungdahlii, Clostridium lortetii, Clostridium lundense, Clostridium magnum, Clostridium malenominatum, Clostridium mangenotii, Clostridium
mayombei, Clostridium methoxybenzovorans, Clostridium methylpentosum, Clostridium neopropionicum, Clostridium nexile, Clostridium nitrophenolicum,
Clostridium novyi, Clostridium oceanicum, Clostridium orbiscindens, Clostridium oroticum, Clostridium oxalicum, Clostridium papyrosolvens, Clostridium
paradoxum, Clostridium paraperfringens (Alias: C. welchii), Clostridium paraputrificum, Clostridium pascui, Clostridium pasteurianum, Clostridium
peptidivorans, Clostridium perenne, Clostridium perfringens, Clostridium pfennigii, Clostridium phytofermentans, Clostridium piliforme, Clostridium
polysaccharolyticum, Clostridium populeti, Clostridium propionicum, Clostridium proteoclasticum, Clostridium proteolyticum, Clostridium psychrophilum,
Clostridium puniceum, Clostridium purinilyticum, Clostridium putrefaciens, Clostridium putrificum, Clostridium quercicolum, Clostridium quinii,Clostridium
ramosum, Clostridium rectum, Clostridium roseum, Clostridium saccharobutylicum, Clostridium saccharogumia, Clostridium saccharolyticum, Clostridium
saccharoperbutylacetonicum, Clostridium sardiniense, Clostridium sartagoforme, Clostridium scatologenes, Clostridium schirmacherense, Clostridium
scindens, Clostridium septicum, Clostridium sordellii, Clostridium sphenoides, Clostridium spiroforme, Clostridium sporogenes, Clostridium
sporosphaeroides, Clostridium stercorarium, Clostridium stercorarium leptospartum, Clostridium stercorarium stercorarium, Clostridium stercorarium
thermolacticum, Clostridium sticklandii, Clostridium straminisolvens, Clostridium subterminale, Clostridium sufflavum, Clostridium sulfidigenes, Clostridium
symbiosum, Clostridium tagluense, Clostridium tepidiprofundi, Clostridium termitidis, Clostridium tertium, Clostridium tetani, Clostridium tetanomorphum,
Clostridium thermaceticum, Clostridium thermautotrophicum, Clostridium thermoalcaliphilum, Clostridium thermobutyricum, Clostridium thermocellum,
Clostridium thermocopriae, Clostridium thermohydrosulfuricum, Clostridium thermolacticum, Clostridium thermopalmarium, Clostridium
thermopapyrolyticum, Clostridium thermosaccharolyticum, Clostridium thermosuccinogenes, Clostridium thermosulfurigenes, Clostridium
thiosulfatireducens, Clostridium tyrobutyricum, Clostridium uliginosum, Clostridium ultunense, Clostridium villosum, Clostridium vincentii, Clostridium
viride, Clostridium xylanolyticum, Clostridium xylanovorans

| | | | | |
|---|---|---|---|---|
| Dactylosporangium | Deinococcus | Delftia | Echinicola | Nesterenkonia |
| Dactylosporangium aurantiacum | Deinococcus aerius | Delftia acidovorans | Echinicola pacifica | Nesterenkonia holobia |
| Dactylosporangium fulvum | Deinococcus apachensis | Desulfovibrio | Echinicola vietnamensis | Nocardia |
| Dactylosporangium matsuzakiense | Deinococcus aquaticus | Desulfovibrio desulfuricans | Flavobacterium | Nocardia argentinensis |
| Dactylosporangium roseum | Deinococcus aquatilis | Diplococcus | Flavobacterium antarcticum | Nocardia corallina |
| Dactylosporangium thailandense | Deinococcus caeni | Diplococcus pneumoniae | Flavobacterium aquatile | Nocardia otitidiscaviarum |
| Dactylosporangium vinaceum | Deinococcus radiodurans | Faecalibacterium | Flavobacterium aquidurense | L. sakei |
| Enterobacter | Deinococcus radiophilus | Faecalibacterium prausnitzii | Flavobacterium balustinum | L. salivarius |
| E. aerogenes | Enterobacter kobei | Fangia | Flavobacterium croceum | L. sanfranciscensis |
| E. amnigenus | E. ludwigii | Fangia hongkongensis | Flavobacterium cucumis | L. satsumensis |
| E. agglomerans | E. mori | Fastidiosipila | Flavobacterium daejeonense | L. secaliphilus |
| E. arachidis | E. nimipressuralis | Fastidiosipila sanguinis | Flavobacterium defluvii | L. sharpeae |
| E. asburiae | E. oryzae | Fusobacterium | Flavobacterium degerlachei | L. siliginis |
| E. cancerogenous | E. pulveris | Fusobacterium nucleatum | Flavobacterium denitrificans | L. spicheri |
| E. cloacae | E. pyrinus | Ideonella | Flavobacterium filum | L. suebicus |
| E. cowanii | E. radicincitans | Ideonella azotifigens | Flavobacterium flevense | L. thailandensis |
| E. dissolvens | E. taylorae | Idiomarina | Flavobacterium frigidarium | L. ultunensis |
| E. gergoviae | E. turicensis | Idiomarina abyssalis | Flavobacterium mizutaii | L. vaccinostercus |
| E. helveticus | E. sakazakii | Idiomarina baltica | Flavobacterium okeanokoites | L. vaginalis |
| | Enterobacter soli | | | |
| E. hormaechei | Enterococcus | Idiomarina fontislapidosi | Janibacter | L. versmoldensis |
| E. intermedius | Enterococcus durans | Idiomarina loihiensis | Janibacter anophelis | L. vini |
| Gaetbulibacter | Enterococcus faecalis | Idiomarina ramblicola | Janibacter corallicola | L. vitulinus |
| Gaetbulibacter saemankumensis | Enterococcus faecium | Idiomarina seosinensis | Janibacter limosus | L. zeae |
| Gallibacterium | Erwinia | Idiomarina zobellii | Janibacter melonis | L. zymae |
| Gallibacterium anatis | Erwinia hapontici | Ignatzschineria | Janibacter terrae | L. gastricus |
| Gallicola | Escherichia | Ignatzschineria larvae | Jannaschia | L. ghanensis |
| Gallicola barnesae | Escherichia coli | Ignavigranum | Jannaschia cystaugens | L. graminis |
| Garciella | Haemophilus | Ignavigranum ruoffiae | Jannaschia helgolandensis | L. hammesii |
| Garciella nitratireducens | Haemophilus aegyptius | Ilumatobacter | Jannaschia pohangensis | L. hamsteri |
| Geobacillus | Haemophilus aphrophilus | Ilumatobacter fluminis | Jannaschia rubra | L. harbinensis |
| Geobacillus thermoglucosidasius | Haemophilus felis | Ilyobacter | Janthinobacterium | L. hayakitensis |
| Geobacillus stearothermophilus | Haemophilus gallinarum | Ilyobacter delafieldii | Janthinobacterium agaricidamnosum | Tatlockia |
| Geobacter | Haemophilus haemolyticus | Ilyobacter insuetus | Janthinobacterium lividum | Tatlockia maceachernii |
| Geobacter bemidjiensis | Haemophilus influenzae | Ilyobacter polytropus | Jejuia | Tatlockia micdadei |
| Geobacter bremensis | Haemophilus paracuniculus | Ilyobacter tartaricus | Jejuia pallidilutea | Tenacibaculum |
| Geobacter chapellei | Haemophilus parahaemolyticus | Listeria ivanovii | Jeotgalibacillus | Tenacibaculum amylolyticum |
| Geobacter grbiciae | Haemophilus parainfluenzae | L. marthii | Jeotgalibacillus alimentarius | Tenacibaculum discolor |
| Geobacter hydrogenophilus | Haemophilus | L. monocytogenes | Jeotgalicoccus | Tenacibaculum gallaicum |
| Geobacter lovleyi | paraphrohaemolyticus | L. newyorkensis | Jeotgalicoccus halotolerans | Tenacibaculum lutimaris |
| Geobacter metallireducens | Haemophilus parasuis | L. riparia | Micrococcus | Tenacibaculum mesophilum |
| Geobacter pelophilus | Haemophilus pittmaniae | L. rocourtiae | Micrococcus luteus | Tenacibaculum skagerrakense |
| Geobacter pickeringii | Hafnia | L. seeligeri | Micrococcus lylae | Tepidanaerobacter |
| Geobacter sulfurreducens | Hafnia alvei | L. weihenstephanensis | Moraxella | Tepidanaerobacter syntrophicus |
| Geodermatophilus | Hahella | L. welshimeri | Moraxella bovis | Tepidibacter |
| Geodermatophilus obscurus | Hahella ganghwensis | Listonella | Moraxella nonliquefaciens | Tepidibacter formicigenes |
| Gluconacetobacter | Halalkalibacillus | Listonella anguillarum | Moraxella osloensis | Tepidibacter thalassicus |
| Gluconacetobacter xylinus | Halalkalibacillus halophilus | Macrococcus | Nakamurella | Thermus |
| Gordonia | Helicobacter | Macrococcus bovicus | Nakamurella multipartita | Thermus aquaticus |
| Gordonia rubripertincta | Helicobacter pylori | Marinobacter | Nannocystis | Thermus filiformis |
| Kaistia | Labedella | Marinobacter algicola | Nannocystis pusilla | Thermus thermophilus |
| Kaistia adipata | Labedella gwakjiensis | Marinobacter bryozoorum | Natranaerobius | Xanthobacter |
| Kaistia soli | Labrenzia | Marinobacter flavimaris | Natranaerobius | Xanthobacter agilis |
| Kangiella | Labrenzia aggregata | Meiothermus | thermophilus | Xanthobacter aminoxidans |
| Kangiella aquimarina | Labrenzia alba | Meiothermus ruber | Natranaerobius trueperi | Xanthobacter autotrophicus |
| Kangiella koreensis | Labrenzia alexandrii | Methylophilus | Naxibacter | Xanthobacter flavus |
| Kerstersia | Labrenzia marina | Methylophilus methylotrophus | Naxibacter alkalitolerans | Xanthobacter tagetidis |
| Kerstersia gyiorum | Labrys | Microbacterium | Neisseria | Xanthobacter viscosus |
| Kiloniella | Labrys methylaminiphilus | Microbacterium ammoniaphilum | Neisseria cinerea | Xanthomonas |
| Kiloniella laminariae | Labrys miyagiensis | Microbacterium arborescens | Neisseria denitrificans | Xanthomonas albilineans |
| Klebsiella | Labrys monachus | Microbacterium liquefaciens | Neisseria gonorrhoeae | Xanthomonas alfalfae |
| K. granulomatis | Labrys okinawensis | Microbacterium oxydans | Neisseria lactamica | Xanthomonas arboricola |
| K. oxytoca | Labrys portucalensis | L. mali | Neisseria mucosa | Xanthomonas axonopodis |
| K. pneumoniae | Lactobacillus | L. manihotivorans | Neisseria sicca | Xanthomonas campestris |
| K. terrigena | [see below] | L. mindensis | Neisseria subflava | Xanthomonas citri |
| K. variicola | Laceyella | L. mucosae | Neptunomonas | Xanthomonas codiaei |
| Kluyvera | Laceyella putida | L. murinus | Neptunomonas japonica | Xanthomonas cucurbitae |
| Kluyvera ascorbata | Lechevalieria | L. nagelii | L. parakefiri | Xanthomonas euvesicatoria |
| Kocuria | Lechevalieria aerocolonigenes | L. namurensis | L. paralimentarius | Xanthomonas fragariae |
| Kocuria roasea | Legionella | L. nantensis | L. paraplantarum | Xanthomonas fuscans |
| Kocuria varians | [see below] | L. oligofermentans | L. pentosus | Xanthomonas gardneri |
| Kurthia | Listeria | L. oris | L. perolens | Xanthomonas hortorum |
| Kurthia zopfii | L. aquatica | L. panis | L. plantarum | Xanthomonas hyacinthi |
| Lactobacillus | L. booriae | L. pantheris | L. pontis | Xanthomonas perforans |
| L. acetotolerans | L. cornellensis | L. parabrevis | L. protectus | Xanthomonas phaseoli |
| L. acidifarinae | L. fleischmannii | L. parabuchneri | L. psittaci | Xanthomonas pisi |
| L. acidipiscis | L. floridensis | L. paracasei | L. rennini | Xanthomonas populi |
| L. acidophilus | L. grandensis | L. paracollinoides | L. reuteri | Xanthomonas theicola |
| Lactobacillus agilis | L. grayi | L. parafarraginis | L. rhamnosus | Xanthomonas translucens |
| L. algidus | L. innocua | L. homohiochii | L. rimae | Xanthomonas vesicatoria |
| L. alimentarius | L. catenaformis | L. iners | L. rogosae | Xylella |
| L. amylolyticus | L. ceti | L. ingluviei | L. rossiae | Xylella fastidiosa |
| L. amylophilus | L. coleohominis | L. intestinalis | L. ruminis | Xylophilus |
| L. amylotrophicus | L. collinoides | L. fuchuensis | L. saerimneri | Xylophilus ampelinus |
| L. amylovorus | L. composti | L. gallinarum | L. jensenii | Zobellella |
| L. animalis | L. concavus | L. gasseri | L. johnsonii | Zobellella denitrificans |
| L. antri | L. coryniformis | Candidatus Legionella jeonii | L. kalixensis | Zobellella taiwanensis |
| L. apodemi | L. crispatus | Legionella jordanis | L. kefiranofaciens | Zeaxanthinibacter |
| L. aviarius | L. crustorum | Legionella lansingensis | L. kefiri | Zeaxanthinibacter enoshimensis |
| L. bifermentans | L. curvatus | Legionella londiniensis | L. kimchii | Zhihengliuella |
| L. brevis | L. delbrueckii subsp. | Legionella longbeachae | L. helveticus | Zhihengliuella halotolerans |
| | bulgaricus | | | |
| L. buchneri | L. delbrueckii subsp. | Legionella lytica | L. hilgardii | Xylanibacterium |
| | delbrueckii | | | |
| L. camelliae | L. delbrueckii subsp. | Legionella maceachernii | Legionella quinlivanii | Xylanibacterium ulmi |
| | lactis | | | |
| L. casei | L. dextrinicus | Legionella massiliensis | Legionella rowbothamii | |
| L. kitasatonis | L. diolivorans | Legionella micdadei | Legionella rubrilucens | |
| L. kunkeei | L. equi | Legionella monrovica | Legionella sainthelensi | |
| L. leichmannii | L. equigenerosi | Legionella moravica | Legionella santicrucis | |
| L. lindneri | L. farraginis | Legionella nagasakiensis | Legionella shakespearei | |
| L. malefermentans | L. farciminis | Legionella nautarum | Legionella spiritensis | |
| Legionella | L. fermentum | Legionella norrlandica | Legionella steelei | |
| Legionella adelaidensis | L. fornicalis | Legionella oakridgensis | Legionella steigerwaltii | |
| Legionella anisa | L. fructivorans | Legionella parisiensis | Legionella taurinensis | |
| Legionella beliardensis | L. frumenti | Legionella pittsburghensis | Legionella tucsonensis | |
| Legionella birminghamensis | Legionella drancourtii | Legionella pneumophila | Legionella tunisiensis | |
| Legionella bozemanae | Legionella dresdenensis | Legionella quateirensis | Legionella wadsworthii | |
| Legionella brunensis | Legionella drozanskii | Prevotella | Legionella waltersii | |
| Legionella busanensis | Legionella dumoffii | Prevotella albensis | Legionella worsleiensis | |
| Legionella cardiaca | Legionella erythra | Prevotella amnii | Legionella yabuuchiae | |
| Legionella cherrii | Legionella fairfieldensis | Prevotella bergensis | Quadrisphaera | |
| Legionella cincinnatiensis | Legionella fallonii | Prevotella bivia | Quadrisphaera granulorum | |
| Legionella clemsonensis | Legionella feeleii | Prevotella brevis | Quatrionicoccus | |
| Legionella donaldsonii | Legionella geestiana | Prevotella bryantii | Quatrionicoccus | |
| Oceanibulbus | Legionella genomospecies | Prevotella buccae | australiensis | |
| Oceanibulbus indolifex | Legionella gormanii | Prevotella buccalis | Quinella | |
| Oceanicaulis | Legionella gratiana | Prevotella copri | Quinella ovalis | |
| Oceanicaulis alexandrii | Legionella gresilensis | Prevotella dentalis | Ralstonia | |
| Oceanicola | Legionella hackeliae | Prevotella denticola | Ralstonia eutropha | |
| Oceanicola batsensis | Legionella impletisoli | Prevotella disiens | Ralstonia insidiosa | |
| Oceanicola granulosus | Legionella israelensis | Prevotella histicola | Ralstonia mannitolilytica | |
| Oceanicola nanhaiensis | Legionella jamestowniensis | Prevotella intermedia | Ralstonia pickettii | |
| Oceanimonas | Paenibacillus | Prevotella maculosa | Ralstonia pseudosolanacearum | |
| Oceanimonas baumannii | Paenibacillus thiaminolyticus | Prevotella marshii | Ralstonia syzygii | |
| Oceaniserpentilla | Pantoea | Prevotella melaninogenica | Ralstonia solanacearum | |
| Oceaniserpentilla haliotis | Pantoea agglomerans | Prevotella micans | Ramlibacter | |
| Oceanisphaera | Paracoccus | Prevotella multiformis | Ramlibacter henchirensis | |
| Oceanisphaera donghaensis | Paracoccus alcaliphilus | Prevotella nigrescens | Ramlibacter tataouinensis | |
| Oceanisphaera litoralis | Paucimonas | Prevotella oralis | Raoultella | |
| Oceanithermus | Paucimonas lemoignei | Prevotella oris | Raoultella ornithinolytica | |
| Oceanithermus desulfurans | Pectobacterium | Prevotella oulorum | Raoultella planticola | |
| Oceanithermus profundus | Pectobacterium aroidearum | Prevotella pallens | Raoultella terrigena | |
| Oceanobacillus | Pectobacterium atrosepticum | Prevotella salivae | Rathayibacter | |
| Oceanobacillus caeni | Pectobacterium betavasculorum | Prevotella stercorea | Rathayibacter caricis | |
| Oceanospirillum | Pectobacterium cacticida | Prevotella tannerae | Rathayibacter festucae | |
| Oceanospirillum linum | Pectobacterium carnegieana | Prevotella timonensis | Rathayibacter iranicus | |
| Saccharococcus | Pectobacterium carotovorum | Prevotella veroralis | Rathayibacter rathayi | |
| Saccharococcus thermophilus | Pectobacterium chrysanthemi | Providencia | Rathayibacter toxicus | |
| Saccharomonospora | Pectobacterium cypripedii | Providencia stuartii | Rathayibacter tritici | |
| Saccharomonospora azurea | Pectobacterium rhapontici | Pseudomonas | Rhodobacter | |
| Saccharomonospora cyanea | Pectobacterium wasabiae | Pseudomonas aeruginosa | Rhodobacter sphaeroides | |
| Saccharomonospora viridis | Planococcus | Pseudomonas alcaligenes | Ruegeria | |
| Saccharophagus | Planococcus citreus | Pseudomonas anguillispetica | Ruegeria gelatinovorans | |
| Saccharophagus degradans | Planomicrobium | Pseudomonas fluorescens | Stenotrophomonas | |
| Saccharopolyspora | Planomicrobium okeanokoites | Pseudoalteromonas haloplanktis | Stenotrophomonas | |
| Saccharopolyspora erythraea | Plesiomonas | Pseudomonas mendocina | maltophilia | |
| Saccharopolyspora gregorii | Plesiomonas shigelloides | Pseudomonas pseudoalcaligenes | Streptococcus | |
| Saccharopolyspora hirsuta | Proteus | Pseudomonas putida | [also see below] | |
| Saccharopolyspora hordei | Proteus vulgaris | Pseudomonas tutzeri | Streptomyces | |
| Saccharopolyspora rectivirgula | Sagittula | Pseudomonas syringae | Streptomyces achromogenes | |
| Saccharopolyspora spinosa | Sagittula stellata | Psychrobacter | Streptomyces cesalbus | |
| Saccharopolyspora taberi | Salegentibacter | Psychrobacter faecalis | Streptomyces cescaepitosus | |
| Saccharothrix | Salegentibacter salegens | Psychrobacter phenylpyruvicus | Streptomyces cesdiastaticus | |
| Saccharothrix australiensis | Salimicrobium | Sanguibacter | Streptomyces cesexfoliatus | |
| Saccharothrix coeruleofusca | Salimicrobium album | Sanguibacter keddieii | Streptomyces fimbriatus | |
| Saccharothrix espanaensis | Salinibacter | Sanguibacter suarezii | Streptomyces fradiae | |
| Saccharothrix longispora | Salinibacter ruber | Saprospira | Streptomyces fulvissimus | |
| Saccharothrix mutabilis | Salinicoccus | Saprospira grandis | Streptomyces griseoruber | |
| Saccharothrix syringae | Salinicoccus alkaliphilus | Sarcina | Streptomyces griseus | |
| Saccharothrix tangerinus | Salinicoccus hispanicus | Sarcina maxima | Streptomyces lavendulae | |
| Saccharothrix texasensis | Salinicoccus roseus | Sarcina ventriculi | Streptomyces phaeochromogenes | |
| Staphylococcus | Salinispora | Sebaldella | Streptomyces thermodiastaticus | |
| S. arlettae | Salinispora arenicola | Sebaldella termitidis | Streptomyces tubercidicus | |
| S. agnetis | Salinispora tropica | Serratia | S. schleiferi | |
| S. aureus | Salinivibrio | Serratia fonticola | S. sciuri | |
| S. auricularis | Salinivibrio costicola | Serratia marcescens | S. simiae | |
| S. capitis | Salmonella | Sphaerotilus | S. simulans | |
| S. caprae | Salmonella bongori | Sphaerotilus natans | S. stepanovicii | |
| S. carnosus | Salmonella enterica | Sphingobacterium | S. succinus | |
| S. caseolyticus | Salmonella subterranea | Sphingobacterium multivorum | S. vitulinus | |
| S. chromogenes | Salmonella typhi | Staphylococcus | S. warneri | |
| S. cohnii | S. equorum | [see below] | S. xylosus | |
| S. condimenti | S. felis | S. microti | Streptococcus thermophilus | |
| S. delphini | S. fleurettii | S. muscae | Streptococcus sanguinis | |
| S. devriesei | S. gallinarum | S. nepalensis | Streptococcus sobrinus | |
| S. epidermidis | S. haemolyticus | S. pasteuri | Streptococcus suis | |
| Streptococcus | S. hominis | S. petrasii | Streptococcus uberis | |
| Streptococcus agalactiae | S. hyicus | S. pettenkoferi | Streptococcus vestibularis | |
| Streptococcus anginosus | S. intermedius | S. piscifermentans | Streptococcus viridans | |
| Streptococcus bovis | S. kloosii | S. pseudintermedius | Streptococcus zooepidemicus | |
| Streptococcus canis | S. leei | S. pseudolugdunensis | Virgibacillus | |
| Streptococcus constellatus | S. lentus | S. pulvereri | Virgibacillus halodenitrificans | |
| Streptococcus downei | S. lugdunensis | S. rostri | Virgibacillus pantothenticus | |
| Streptococcus dysgalactiae | S. lutrae | S. saccharolyticus | Weissella | |
| Streptococcus equines | S. lyticans | S. saprophyticus | Weissella cibaria | |
| Streptococcus faecalis | S. massiliensis | Streptococcus orisratti | Weissella confusa | |
| Streptococcus ferus | Streptococcus infantarius | Streptococcus parasanguinis | Weissella halotolerans | |
| Uliginosibacterium | Streptococcus iniae | Streptococcus peroris | Weissella hellenica | |
| Uliginosibacterium gangwonense | Streptococcus intermedius | Streptococcus pneumoniae | Weissella kandleri | |
| Ulvibacter | Streptococcus lactarius | Streptococcus pseudopneumoniae | Weissella koreensis | |
| Ulvibacter litoralis | Streptococcus milleri | Streptococcus pyogenes | Weissella minor | |
| Umezawaea | Streptococcus mitis | Streptococcus ratti | Weissella paramesenteroides | |
| Umezawaea tangerina | Streptococcus mutans | Streptococcus salivariu | Weissella soli | |
| Undibacterium | Streptococcus oralis | Vibrio | Weissella thailandensis | |
| Undibacterium pigrum | Streptococcus tigurinus | Vibrio aerogenes | Weissella viridescens | |
| Ureaplasma | Vagococcus | Vibrio aestuarianus | Williamsia | |
| Ureaplasma urealyticum | Vagococcus carniphilus | Vibrio albensis | Williamsia marianensis | |
| Ureibacillus | Vagococcus elongatus | Vibrio alginolyticus | Williamsia maris | |
| Ureibacillus composti | Vagococcus fessus | Vibrio campbellii | Williamsia serinedens | |
| Ureibacillus suwonensis | Vagococcus fluvialis | Vibrio cholerae | Winogradskyella | |
| Ureibacillus terrenus | Vagococcus lutrae | Vibrio cincinnatiensis | Winogradskyella thalassocola | |
| Ureibacillus thermophilus | Vagococcus salmoninarum | Vibrio coralliilyticus | Wolbachia | |
| Ureibacillus thermosphaericus | Variovorax | Vibrio cyclitrophicus | Wolbachia persica | |
| Xenophilus | Variovorax boronicumulans | Vibrio diazotrophicus | Wolinella | |
| Xenophilus azovorans | Variovorax dokdonensis | Vibrio fluvialis | Wolinella succinogenes | |
| Xenorhabdus | Variovorax paradoxus | Vibrio furnissii | Zobellia | |
| Xenorhabdus beddingii | Variovorax soli | Vibrio gazogenes | Zobellia galactanivorans | |
| Xenorhabdus bovienii | Veillonella | Vibrio halioticoli | Zobellia uliginosa | |
| Xenorhabdus cabanillasii | Veillonella atypica | Vibrio harveyi | Zoogloea | |
| Xenorhabdus doucetiae | Veillonella caviae | Vibrio ichthyoenteri | Zoogloea ramigera | |
| Xenorhabdus griffiniae | Veillonella criceti | Vibrio mediterranei | Zoogloea resiniphila | |
| Xenorhabdus hominickii | Veillonella dispar | Vibrio metschnikovii | Zooshikella | |
| Xenorhabdus koppenhoeferi | Veillonella montpellierensis | Vibrio mytili | Zooshikella ganghwensis | |
| Xenorhabdus nematophila | Veillonella parvula | Vibrio natriegens | Zunongwangia | |
| Xenorhabdus poinarii | Veillonella ratti | Vibrio navarrensis | Zunongwangia profunda | |
| Xylanibacter | Veillonella rodentium | Vibrio nereis | Zymobacter | |
| Xylanibacter oryzae | Venenivibrio | Vibrio nigripulchritudo | Zymobacter palmae | |
| | Venenivibrio stagnispumantis | Vibrio ordalii | Zymomonas | |
| | Verminephrobacter | Vibrio orientalis | Zymomonas mobilis | |
| | Verminephrobacter eiseniae | Vibrio parahaemolyticus | Zymophilus | |
| | Verrucomicrobium | Vibrio pectenicida | Zymophilus paucivorans | |
| | Verrucomicrobium spinosum | Vibrio penaeicida | Zymophilus raffinosivorans | |
| | Yangia | Vibrio proteolyticus | | |
| | Yangia pacifica | Vibrio shilonii | | |
| | Yaniella | Vibrio splendidus | | |
| | Yaniella flava | Vibrio tubiashii | | |
| | Yaniella halotolerans | Vibrio vulnificus | | |
| | Yeosuana | Yersinia mollaretii | | |
| | Yeosuana aromativorans | Yersinia philomiragia | | |
| | Yersinia | Yersinia pestis | | |
| | Yersinia aldovae | Yersinia pseudotuberculosis | | |
| | Yersinia bercovieri | Yersinia rohdei | | |
| | Yersinia enterocolitica | Yersinia pseudotuberculosis | | |
| | Yersinia entomophaga | Yersinia ruckeri | | |
| | Yersinia frederiksenii | Yokenella | | |
| | Yersinia intermedia | Yokenella regensburgei | | |
| | Yersinia kristensenii | Yonghaparkia | | |
| | | Yonghaparkia alkaliphila | | |
| | | Zavarzinia | | |
| | | Zavarzinia compransoris | | |

TABLE 3
Pseudomonas Species & Strains Comprising PSPTO_0477 or an Orthologue of PSPTO_0477
Pseudomonas Species & Strains Comprising an Orthologue of PSPTO_0477 obtained by the BLASTN
comparison with non-reductant nucleotide database in NCBI.

| | Max | Total | Query | | Per. | |
|---|---|---|---|---|---|---|
| Description | Score | Score | Cover | E value | Ident | Accession |
| Pseudomonas syringae pv. tomato strain delta IV, IX | 2660 | 2660 | 100% | 0 | 100% | CP047072.1 |
| chromosome | | | | | | |
| Pseudomonas syringae pv. tomato strain delta VI | 2660 | 2660 | 100% | 0 | 100% | CP047071.1 |
| chromosome | | | | | | |
| Pseudomonas syringae pv. tomato strain delta X | 2660 | 2660 | 100% | 0 | 100% | CP047073.1 |
| chromosome, complete genome | | | | | | |
| Pseudomonas syringae pv. tomato str. DC3000, | 2660 | 2660 | 100% | 0 | 100% | AE016853.1 |
| complete genome | | | | | | |
| Pseudomonas syringae strain Ps25 chromosome | 2615 | 2615 | 100% | 0 | 99% | CP034558.1 |
| Pseudomonas syringae pv. tomato strain B13-200 | 2615 | 2615 | 100% | 0 | 99% | CP019871.1 |
| chromosome, complete genome | | | | | | |
| Pseudomonas syringae pv. avii isolate CFBP3846 | 2577 | 2577 | 100% | 0 | 99% | LT963402.1 |
| genome assembly, chromosome: 1 | | | | | | |
| Pseudomonas avellanae strain R2leaf chromosome, | 2316 | 2316 | 100% | 0 | 96% | CP026562.1 |
| complete genome | | | | | | |
| Pseudomonas syringae group genomosp. 3 isolate | 2316 | 2316 | 100% | 0 | 96% | LT963408.1 |
| CFBP6411 genome assembly, chromosome: I | | | | | | |
| Pseudomonas syringae pv. actinidiae str. | 2305 | 2305 | 100% | 0 | 96% | CP032631.1 |
| Shaanxi_M228 chromosome, complete genome | | | | | | |
| Pseudomonas syringae pv. actinidiae strain P155 | 2305 | 2305 | 100% | 0 | 96% | CP032871.1 |
| chromosome, complete genome | | | | | | |
| Pseudomonas syringae pv. actinidiae strain | 2305 | 2305 | 100% | 0 | 96% | CP024712.1 |
| MAFF212063 chromosome, complete genome | | | | | | |
| Pseudomonas syringae pv. actinidiae strain CRAFRU | 2305 | 2305 | 100% | 0 | 96% | CP019732.1 |
| 14.08, complete genome | | | | | | |
| Pseudomonas syringae pv. actinidiae strain CRAFRU | 2305 | 2305 | 100% | 0 | 96% | CP019730.1 |
| 12.29, complete genome | | | | | | |
| Pseudomonas syringae pv. actinidiae ICMP 9853, | 2305 | 2305 | 100% | 0 | 96% | CP018202.1 |
| complete genome | | | | | | |
| Pseudomonas syringae pv. actinidiae strain NZ-47, | 2305 | 2305 | 100% | 0 | 96% | CP017009.1 |
| complete genome | | | | | | |
| Pseudomonas syringae pv. actinidiae strain NZ-45, | 2305 | 2305 | 100% | 0 | 96% | CP017007.1 |
| complete genome | | | | | | |
| Pseudomonas syringae pv. actinidiae ICMP 18884, | 2305 | 2305 | 100% | 0 | 96% | CP011972.2 |
| complete genome | | | | | | |
| Pseudomonas syringae pv. actinidiae ICMP 18708, | 2305 | 2305 | 100% | 0 | 96% | CP012179.1 |
| complete genome | | | | | | |
| Pseudomonas syringae pv. maculicola str. ES4326 | 1951 | 1951 | 100% | 0 | 91% | CP047260.1 |
| chromosome, complete genome | | | | | | |
| Pseudomonas coronafaciens strain X-1 chromosome, | 1912 | 1912 | 100% | 0 | 91% | CP050260.1 |
| complete genome | | | | | | |
| Pseudomonas coronafaciens pv. coronafaciens strain | 1912 | 1912 | 100% | 0 | 91% | CP046441.1 |
| B19001 chromosome, complete genome | | | | | | |
| Pseudomonas coronafaciens pv. oryzae str. 1_6 | 1895 | 1895 | 100% | 0 | 90% | CP046035.1 |
| chromosome, complete genome | | | | | | |
| Pseudomonas syringae CC1557, complete sequence | 1857 | 1857 | 100% | 0 | 90% | CP007014.1 |
| Pseudomonas syringae strain 31R1 genome assembly, | 1845 | 1845 | 100% | 0 | 90% | LT629769.1 |
| chromosome: I | | | | | | |
| Pseudomonas cerasi isolate PL963 genome assembly, | 1829 | 1829 | 100% | 0 | 90% | LT963395.1 |
| chromosome: 1 | | | | | | |
| Pseudomonas sp. 58 isolate Sour cherry (Prunus | 1829 | 1829 | 100% | 0 | 90% | LT222319.1 |
| cerasus) symptomatic leaf genome assembly, | | | | | | |
| chromosome: 1 | | | | | | |
| Pseudomonas syringae UMAF0158, complete genome | 1829 | 1829 | 100% | 0 | 90% | CP005970.1 |
| Pseudomonas syringae pv. syringae strain Pss9097 | 1823 | 1823 | 100% | 0 | 90% | CP026568.1 |
| chromosome, complete genome | | | | | | |
| Pseudomonas syringae UB303 chromosome, complete | 1812 | 1812 | 100% | 0 | 89% | CP047267.1 |
| genome | | | | | | |
| Pseudomonas syringae USA011 chromosome, complete | 1812 | 1812 | 100% | 0 | 89% | CP045799.1 |
| genome | | | | | | |
| Pseudomonas syringae pv. syringae isolate CFBP4215 | 1812 | 1812 | 100% | 0 | 89% | LT962480.1 |
| genome assembly, chromosome: 1 | | | | | | |
| Pseudomonas syringae pv. syringae B728a, complete | 1812 | 1812 | 100% | 0 | 89% | CP000075.1 |
| genome | | | | | | |
| Pseudomonas sp. KUIN-1 DNA, complete genome | 1807 | 1807 | 100% | 0 | 89% | AP020337.1 |
| Pseudomonas syringae pv. syringae B301D, complete | 1807 | 1807 | 100% | 0 | 89% | CP005969.1 |
| genome | | | | | | |
| Pseudomonas syringae pv. syringae isolate CFBP2118 | 1801 | 1801 | 100% | 0 | 89% | LT962481.1 |
| genome assembly, chromosome: 1 | | | | | | |
| Pseudomonas amygdali pv. tabaci str. ATCC 11528 | 1790 | 1790 | 100% | 0 | 89% | CP042804.1 |
| chromosome, complete genome | | | | | | |
| Pseudomonas syringae strain CFBP 2116 genome | 1762 | 1762 | 100% | 0 | 89% | LT985192.1 |
| assembly, chromosome: 1 | | | | | | |
| Pseudomonas amygdali pv. morsprunorum strain | 1762 | 1762 | 100% | 0 | 89% | CP026558.1 |
| R15244 chromosome, complete genome | | | | | | |
| Pseudomonas syringae isolate CFBP3840 genome | 1762 | 1762 | 100% | 0 | 89% | LT963409.1 |
| assembly, chromosome: 1 | | | | | | |
| Pseudomonas sp. KBS0707 chromosome, complete | 1757 | 1757 | 100% | 0 | 89% | CP041754.1 |
| genome | | | | | | |
| Pseudomonas savastanoi pv. savastanoi NCPPB 3335, | 1757 | 1757 | 100% | 0 | 89% | CP008742.1 |
| complete genome | | | | | | |
| Pseudomonas amygdali pv. lachrymans strain NM002, | 1757 | 1757 | 100% | 0 | 89% | CP020351.1 |
| complete genome | | | | | | |
| Pseudomonas savastanoi pv. phaseolicola 1448A | 1757 | 1757 | 100% | 0 | 89% | CP000058.1 |
| chromosome, complete genome | | | | | | |
| Pseudomonas amygdali pv. lachrymans str. M301315 | 1751 | 1751 | 100% | 0 | 89% | CP031225.1 |
| chromosome, complete genome | | | | | | |
| Pseudomonas syringae pv. cerasicola isolate CFBP6109 | 1751 | 1751 | 100% | 0 | 89% | LT963391.1 |
| genome assembly, chromosome: 1 | | | | | | |
| Pseudomonas syringae pv. pisi str. PP1 chromosome, | 1746 | 1746 | 100% | 0 | 89% | CP034078.1 |
| complete genome | | | | | | |
| Pseudomonas syringae pv. lapsa strain ATCC 10859, | 1740 | 1740 | 100% | 0 | 88% | CP013183.1 |
| complete genome | | | | | | |
| Pseudomonas syringae pv. syringae HS191, complete | 1724 | 1724 | 100% | 0 | 88% | CP006256.1 |
| genome | | | | | | |
| Pseudomonas syringae pv. atrofaciens strain LMG5095 | 1712 | 1712 | 100% | 0 | 88% | CP028490.1 |
| chromosome, complete genome | | | | | | |
| Pseudomonas viridiflava strain CFBP 1590 genome | 1602 | 1602 | 100% | 0 | 87% | LT855380.1 |
| assembly, chromosome: I | | | | | | |
| Pseudomonas asturiensis strain CC1524 chromosome, | 1568 | 1568 | 100% | 0 | 86% | CP047265.1 |
| complete genome | | | | | | |
| Pseudomonas cichorii JBC1, complete genome | 1544 | 1544 | 99% | 0 | 86% | CP007039.1 |
| Paucimonas lemoignei strain NCTC10937 genome | 1447 | 1447 | 100% | 0 | 85% | LS483371.1 |
| assembly, chromosome: 1 | | | | | | |
| Pseudomonas sp. StFLB209 DNA, complete genome | 1203 | 1203 | 93% | 0 | 83% | AP014637.1 |
| Pseudomonas putida strain PP112420, complete | 1155 | 1155 | 99% | 0 | 81% | CP017073.1 |
| genome | | | | | | |
| Pseudomonas putida GB-1 chromosome, complete | 1155 | 1155 | 99% | 0 | 81% | CP000926.1 |
| genome | | | | | | |
| Pseudomonas sp. MRSN12121, complete genome | 1151 | 1151 | 97% | 0 | 82% | CP010892.1 |
| Pseudomonas chlororaphis subsp. chlororaphis strain | 1146 | 1146 | 97% | 0 | 82% | CP027712.1 |
| DSM 50083 chromosome, complete genome | | | | | | |
| Pseudomonas sp. 09C 129 chromosome | 1146 | 1146 | 97% | 0 | 82% | CP025261.1 |
| Pseudomonas chlororaphis strain PCL1606, complete | 1140 | 1140 | 96% | 0 | 82% | CP011110.1 |
| genome | | | | | | |
| Pseudomonas chlororaphis subsp. aureofaciens strain | 1134 | 1134 | 97% | 0 | 81% | CP027721.1 |
| ChPhzTR36 chromosome, complete genome | | | | | | |
| Pseudomonas chlororaphis strain TAMOak81 | 1133 | 1133 | 97% | 0 | 81% | CP027713.1 |
| chromosome, complete genome | | | | | | |
| Pseudomonas chlororaphis subsp. aurantiaca strain | 1129 | 2159 | 98% | 0 | 81% | CP009290.1 |
| JD37, complete genome | | | | | | |
| Pseudomonas chlororaphis subsp. aurantiaca strain K27 | 1123 | 1123 | 97% | 0 | 81% | CP027745.1 |
| chromosome, complete genome | | | | | | |
| Pseudomonas chlororaphis subsp. aurantiaca strain | 1123 | 2072 | 97% | 0 | 81% | CP027744.1 |
| M71 chromosome, complete genome | | | | | | |
| Pseudomonas chlororaphis subsp. aurantiaca strain | 1123 | 1123 | 97% | 0 | 81% | CP027743.1 |
| CW2 chromosome, complete genome | | | | | | |
| Pseudomonas chlororaphis subsp. aurantiaca strain | 1123 | 1123 | 97% | 0 | 81% | CP027715.1 |
| M12 chromosome, complete genome | | | | | | |
| Pseudomonas chlororaphis isolate 189, complete | 1123 | 1123 | 96% | 0 | 81% | CP014867.1 |
| genome | | | | | | |
| Pseudomonas chlororaphis subsp. aurantiaca strain B- | 1118 | 1118 | 97% | 0 | 81% | CP050510.1 |
| 162 chromosome | | | | | | |
| Pseudomonas chlororaphis subsp. aurantiaca strain | 1118 | 1118 | 97% | 0 | 81% | CP027717.1 |
| PCM 2210 chromosome, complete genome | | | | | | |
| Pseudomonas chlororaphis subsp. aurantiaca strain | 1118 | 1118 | 97% | 0 | 81% | CP027746.1 |
| DSM 19603 chromosome, complete genome | | | | | | |
| Pseudomonas chlororaphis subsp. aurantiaca strain 464 | 1118 | 1118 | 97% | 0 | 81% | CP027742.1 |
| chromosome, complete genome | | | | | | |
| Pseudomonas chlororaphis subsp. aurantiaca strain 449 | 1118 | 1118 | 97% | 0 | 81% | CP027741.1 |
| chromosome, complete genome | | | | | | |
| Pseudomonas chlororaphis strain ATCC 17415 | 1118 | 1118 | 97% | 0 | 81% | CP027714.1 |
| chromosome, complete genome | | | | | | |
| Pseudomonas chlororaphis strain LMG 21630 genome | 1118 | 1118 | 96% | 0 | 81% | LT629747.1 |
| assembly, chromosome: I | | | | | | |
| Pseudomonas chlororaphis strain UFB2, complete | 1114 | 1114 | 97% | 0 | 81% | CP011020.1 |
| genome | | | | | | |
| Pseudomonas chlororaphis subsp. aurantiaca strain zm- | 1112 | 1112 | 97% | 0 | 81% | CP048051.1 |
| 1 chromosome, complete genome | | | | | | |
| Pseudomonas chlororaphis subsp. aurantiaca strain ARS | 1112 | 1112 | 97% | 0 | 81% | CP045221.1 |
| 38 chromosome, complete genome | | | | | | |
| Pseudomonas chlororaphis strain B25 chromosome, | 1112 | 1112 | 97% | 0 | 81% | CP027753.1 |
| complete genome | | | | | | |
| Pseudomonas chlororaphis strain Pb-St2 chromosome, | 1112 | 1112 | 97% | 0 | 81% | CP027716.1 |
| complete genome | | | | | | |
| Pseudomonas chlororaphis subsp. aurantiaca DNA, | 1112 | 1112 | 97% | 0 | 81% | AP014623.1 |
| complete genome, strain: StFRB508 | | | | | | |
| Pseudomonas chlororaphis subsp. aureofaciens strain | 1107 | 1107 | 97% | 0 | 81% | CP027722.1 |
| C50 chromosome, complete genome | | | | | | |
| Pseudomonas chlororaphis subsp. aurantiaca strain | 1107 | 1107 | 97% | 0 | 81% | CP027718.1 |
| Q16 chromosome, complete genome | | | | | | |
| Pseudomonas chlororaphis subsp. piscium strain DSM | 1107 | 1107 | 97% | 0 | 81% | CP027707.1 |
| 21509 chromosome, complete genome | | | | | | |
| Pseudomonas chlororaphis strain Lzh-T5 chromosome, | 1107 | 1107 | 97% | 0 | 81% | CP025309.1 |
| complete genome | | | | | | |
| Pseudomonas chlororaphis strain DSM 21509 genome | 1107 | 1107 | 96% | 0 | 81% | LT629761.1 |
| assembly, chromosome: I | | | | | | |
| Pseudomonas chlororaphis subsp. aureofaciens strain | 1096 | 1096 | 97% | 0 | 81% | CP027720.1 |
| DSM 6698 chromosome, complete genome | | | | | | |
| Pseudomonas chlororaphis strain ATCC 13985 genome | 1096 | 1096 | 96% | 0 | 81% | LT629738.1 |
| assembly, chromosome: I | | | | | | |
| Pseudomonas sp. R32 chromosome, complete genome | 1090 | 1090 | 97% | 0 | 81% | CP019396.1 |
| Pseudomonas putida strain 1290 chromosome, | 1090 | 1090 | 99% | 0 | 81% | CP039371.1 |
| complete genome | | | | | | |
| Pseudomonas chlororaphis subsp. piscium strain | 1090 | 1090 | 97% | 0 | 81% | CP027711.1 |
| ChPhzTR44 chromosome, complete genome | | | | | | |
| Pseudomonas chlororaphis subsp. piscium strain | 1090 | 1090 | 97% | 0 | 81% | CP027740.1 |
| ChPhzS140 chromosome, complete genome | | | | | | |
| Pseudomonas chlororaphis subsp. piscium strain ToZa7 | 1090 | 1090 | 97% | 0 | 81% | CP027739.1 |
| chromosome, complete genome | | | | | | |
| Pseudomonas chlororaphis subsp. piscium strain | 1090 | 1090 | 97% | 0 | 81% | CP027738.1 |
| ChPhzS135 chromosome, complete genome | | | | | | |
| Pseudomonas chlororaphis subsp. piscium strain | 1090 | 1090 | 97% | 0 | 81% | CP027737.1 |
| PCL1607 chromosome, complete genome | | | | | | |
| Pseudomonas chlororaphis subsp. piscium strain | 1090 | 1090 | 97% | 0 | 81% | CP027736.1 |
| PCL1391 chromosome, complete genome | | | | | | |
| Pseudomonas chlororaphis subsp. piscium strain | 1090 | 1090 | 97% | 0 | 81% | CP027735.1 |
| DTR133 chromosome, complete genome | | | | | | |
| Pseudomonas chlororaphis subsp. piscium strain | 1090 | 1090 | 97% | 0 | 81% | CP027710.1 |
| SLPH10 chromosome, complete genome | | | | | | |
| Pseudomonas putida S13.1.2, complete genome | 1090 | 1090 | 99% | 0 | 81% | CP010979.1 |

TABLE 4
Non-Pseudomonas Species & Strains Comprising an Orthologue of PSPTO_0477 (BLASTN Results)

| | | Total | Query | E | Per. | |
|---|---|---|---|---|---|---|
| Description | Max Score | Score | Cover | value | Ident | Accession |
| Paucimonas lemoignei strain NCTC10937 genome assembly, chromosome: 1 | 1447 | 1447 | 100% | 0 | 85% | LS483371.1 |
| Stenotrophomonas rhizophila strain GA1 chromosome, complete genome | 1018 | 1018 | 98% | 0 | 80% | CP031729.1 |
| Enterococcus faecalis strain V583 genome | 996 | 996 | 98% | 0 | 79% | CP022312.1 |
| Uncultured bacterium 182_02_C03 genomic sequence | 736 | 736 | 77% | 0 | 79% | KJ802934.1 |

TABLE 5
Pseudomonas Species & Strains Comprising PSPTO_820 or an Orthologue of PSPTO_820 (BLASTN Results)

| | Max | Total | Query | E | Per. | |
|---|---|---|---|---|---|---|
| Description | Score | Score | Cover | value | Ident | Accession |
| Pseudomonas syringae pv. tomato str. DC3000, complete genome | 5746 | 5746 | 100% | 0 | 100% | AE016853.1 |
| Pseudomonas syringae pv. tomato strain delta IV, IX chromosome | 5746 | 5746 | 100% | 0 | 100% | CP047072.1 |
| Pseudomonas syringae pv. tomato strain delta VI chromosome | 5746 | 5746 | 100% | 0 | 100% | CP047071.1 |
| Pseudomonas syringae pv. tomato strain delta X chromosome, complete genome | 5746 | 5746 | 100% | 0 | 100% | CP047073.1 |
| Pseudomonas syringae pv. tomato strain B13-200 chromosome, complete genome | 5674 | 5674 | 100% | 0 | 100% | CP019871.1 |
| Pseudomonas syringae strain Ps25 chromosome | 5674 | 5674 | 100% | 0 | 100% | CP034558.1 |
| Pseudomonas syringae pv. avii isolate CFBP3846 genome assembly, chromosome: 1 | 5651 | 5651 | 100% | 0 | 99% | LT963402.1 |
| Pseudomonas syringae pv. actinidiae ICMP 18708, complete genome | 5164 | 5164 | 98% | 0 | 97% | CP012179.1 |
| Pseudomonas syringae pv. actinidiae ICMP 18884, complete genome | 5164 | 5164 | 98% | 0 | 97% | CP011972.2 |
| Pseudomonas syringae pv. actinidiae str. Shaanxi_M228 chromosome, complete | 5164 | 5164 | 98% | 0 | 97% | CP032631.1 |
| genome | | | | | | |
| Pseudomonas syringae pv. actinidiae strain CRAFRU 12.29, complete genome | 5164 | 5164 | 98% | 0 | 97% | CP019730.1 |
| Pseudomonas syringae pv. actinidiae strain CRAFRU 14.08, complete genome | 5164 | 5164 | 98% | 0 | 97% | CP019732.1 |
| Pseudomonas syringae pv. actinidiae strain MAFF212063 chromosome, complete | 5164 | 5164 | 98% | 0 | 97% | CP024712.1 |
| genome | | | | | | |
| Pseudomonas syringae pv. actinidiae strain NZ-45, complete genome | 5164 | 5164 | 98% | 0 | 97% | CP017007.1 |
| Pseudomonas syringae pv. actinidiae strain NZ-47, complete genome | 5164 | 5164 | 98% | 0 | 97% | CP017009.1 |
| Pseudomonas syringae pv. actinidiae strain P155 chromosome, complete genome | 5164 | 5164 | 98% | 0 | 97% | CP032871.1 |
| Pseudomonas syringae pv. actinidiae ICMP 9853, complete genome | 5169 | 5169 | 98% | 0 | 97% | CP018202.1 |
| Pseudomonas avellanae strain R2leaf chromosome, complete genome | 5145 | 5145 | 98% | 0 | 97% | CP026562.1 |
| Pseudomonas syringae group genomosp. 3 isolate CFBP6411 genome assembly, | 5068 | 5068 | 98% | 0 | 97% | LT963408.1 |
| chromosome: I | | | | | | |
| Pseudomonas syringae pv. cerasicola isolate CFBP6109 genome assembly, | 4109 | 4109 | 98% | 0 | 91% | LT963391.1 |
| chromosome: 1 | | | | | | |
| Pseudomonas amygdali pv. morsprunorum strain R15244 chromosome, complete | 4071 | 4071 | 98% | 0 | 91% | CP026558.1 |
| genome | | | | | | |
| Pseudomonas syringae isolate CFBP3840 genome assembly, chromosome: 1 | 4071 | 4071 | 98% | 0 | 91% | LT963409.1 |
| Pseudomonas syringae strain CFBP 2116 genome assembly, chromosome: 1 | 4071 | 4071 | 98% | 0 | 91% | LT985192.1 |
| Pseudomonas amygdali pv. tabaci str. ATCC 11528 chromosome, complete genome | 4048 | 4048 | 98% | 0 | 91% | CP042804.1 |
| Pseudomonas amygdali pv. lachrymans str. M301315 chromosome, complete | 4043 | 4043 | 98% | 0 | 91% | CP031225.1 |
| genome | | | | | | |
| Pseudomonas amygdali pv. lachrymans strain NM002, complete genome | 4043 | 4043 | 98% | 0 | 91% | CP020351.1 |
| Pseudomonas savastanoi pv. savastanoi NCPPB 3335, complete genome | 4039 | 4039 | 98% | 0 | 91% | CP008742.1 |
| Pseudomonas savastanoi pv. phaseolicola 1448A chromosome, complete genome | 4010 | 4010 | 98% | 0 | 90% | CP000058.1 |
| Pseudomonas sp. KBS0707 chromosome, complete genome | 4010 | 4010 | 98% | 0 | 90% | CP041754.1 |
| Pseudomonas syringae pv. maculicola str. ES4326 chromosome, complete genome | 3509 | 3509 | 98% | 0 | 87% | CP047260.1 |
| Pseudomonas syringae CC1557, complete sequence | 3496 | 6991 | 97% | 0 | 87% | CP007014.1 |
| Pseudomonas coronafaciens pv. oryzae str. 1_6 chromosome, complete genome | 3367 | 3367 | 98% | 0 | 87% | CP046035.1 |
| Pseudomonas coronafaciens pv. coronafaciens strain B19001 chromosome, | 3356 | 3356 | 98% | 0 | 87% | CP046441.1 |
| complete genome | | | | | | |
| Pseudomonas coronafaciens strain X-1 chromosome, complete genome | 3356 | 3356 | 98% | 0 | 87% | CP050260.1 |
| Pseudomonas sp. LPH1, complete genome | 2562 | 2562 | 98% | 0 | 82% | CP017290.1 |
| Pseudomonas aeruginosa DSM 50071, complete genome | 2477 | 2477 | 97% | 0 | 81% | CP012001.1 |
| Pseudomonas aeruginosa genome assembly NCTC10332, chromosome: 1 | 2477 | 2477 | 97% | 0 | 81% | LN831024.1 |
| Pseudomonas aeruginosa isolate B10W, complete genome | 2466 | 2466 | 97% | 0 | 81% | CP017969.1 |
| Pseudomonas aeruginosa strain AR_455 chromosome, complete genome | 2466 | 2466 | 97% | 0 | 81% | CP030328.1 |
| Pseudomonas aeruginosa strain Pa58, complete genome | 2466 | 2466 | 97% | 0 | 81% | CP021775.1 |
| Pseudomonas aeruginosa strain PABL048 chromosome, complete genome | 2466 | 2466 | 97% | 0 | 81% | CP039293.1 |
| Pseudomonas aeruginosa strain PASGNDM345, complete genome | 2466 | 2466 | 97% | 0 | 81% | CP020703.1 |
| Pseudomonas aeruginosa strain PASGNDM699, complete genome | 2466 | 2466 | 97% | 0 | 81% | CP020704.1 |
| Pseudomonas aeruginosa strain PB368 chromosome, complete genome | 2466 | 2466 | 97% | 0 | 81% | CP025050.1 |
| Pseudomonas aeruginosa strain PB369 chromosome, complete genome | 2466 | 2466 | 97% | 0 | 81% | CP025049.1 |
| Pseudomonas aeruginosa strain S04 90 genome | 2466 | 2466 | 97% | 0 | 81% | CP011369.1 |
| Pseudomonas aeruginosa strain T2436 chromosome, complete genome | 2466 | 2466 | 97% | 0 | 81% | CP039988.1 |
| Pseudomonas aeruginosa strain 60503 chromosome, complete genome | 2460 | 2460 | 97% | 0 | 81% | CP041774.1 |
| Pseudomonas aeruginosa strain BAMCPA07-48, complete genome | 2460 | 2460 | 97% | 0 | 81% | CP015377.1 |
| Pseudomonas aeruginosa strain NCTC13715 genome assembly, chromosome: 1 | 2460 | 2460 | 97% | 0 | 81% | LR134330.1 |
| Pseudomonas aeruginosa strain ST773 chromosome, complete genome | 2460 | 2460 | 97% | 0 | 81% | CP041945.1 |
| Pseudomonas aeruginosa strain AR_0353 chromosome, complete genome | 2466 | 2466 | 98% | 0 | 81% | CP027172.1 |
| Pseudomonas aeruginosa strain WPB099 chromosome | 2466 | 2466 | 98% | 0 | 81% | CP031878.1 |
| Pseudomonas aeruginosa strain WPB100 chromosome | 2466 | 2466 | 98% | 0 | 81% | CP031877.1 |
| Pseudomonas aeruginosa strain WPB101 chromosome | 2466 | 2466 | 98% | 0 | 81% | CP031876.1 |
| Pseudomonas aeruginosa isolate PA14Or_reads genome assembly, chromosome: | 2455 | 2455 | 97% | 0 | 81% | LT608330.1 |
| PA14OR | | | | | | |
| Pseudomonas aeruginosa strain 243931 chromosome, complete genome | 2455 | 2455 | 97% | 0 | 81% | CP041772.1 |
| Pseudomonas aeruginosa strain 24Pae112 chromosome, complete genome | 2455 | 2455 | 97% | 0 | 81% | CP029605.1 |
| Pseudomonas aeruginosa strain 268 chromosome, complete genome | 2455 | 2455 | 97% | 0 | 81% | CP032761.1 |
| Pseudomonas aeruginosa strain AR_0354 chromosome, complete genome | 2455 | 2455 | 97% | 0 | 81% | CP027171.1 |
| Pseudomonas aeruginosa strain CCUG 51971 chromosome, complete genome | 2455 | 2455 | 97% | 0 | 81% | CP043328.1 |
| Pseudomonas aeruginosa strain E90 chromosome, complete genome | 2455 | 2455 | 97% | 0 | 81% | CP044006.1 |
| Pseudomonas aeruginosa strain FDAARGOS_571 chromosome, complete genome | 2455 | 2455 | 97% | 0 | 81% | CP033833.1 |
| Pseudomonas aeruginosa strain H26023 chromosome, complete genome | 2455 | 2455 | 97% | 0 | 81% | CP033685.1 |
| Pseudomonas aeruginosa strain L10, complete genome | 2455 | 2455 | 97% | 0 | 81% | CP019338.1 |
| Pseudomonas aeruginosa strain MRSN12280 chromosome, complete genome | 2455 | 2455 | 97% | 0 | 81% | CP028162.1 |
| Pseudomonas aeruginosa strain PAK genome assembly, chromosome: 1 | 2455 | 2455 | 97% | 0 | 81% | LR657304.1 |
| Pseudomonas aeruginosa strain W60856, complete genome | 2455 | 2455 | 97% | 0 | 81% | CP008864.2 |
| Pseudomonas aeruginosa UCBPP-PA14 chromosome | 2455 | 2455 | 97% | 0 | 81% | CP034244.1 |
| Pseudomonas aeruginosa UCBPP-PA14, complete genome | 2455 | 2455 | 97% | 0 | 81% | CP000438.1 |
| Pseudomonas salegens strain CECT 8338 genome assembly, chromosome: I | 2462 | 2462 | 97% | 0 | 81% | LT629787.1 |
| Pseudomonas aeruginosa DNA, complete genome, strain: IOMTU 133 | 2449 | 2449 | 97% | 0 | 81% | AP017302.1 |
| Pseudomonas aeruginosa NCGM2.S1 DNA, complete genome | 2455 | 2455 | 98% | 0 | 81% | AP012280.1 |
| Pseudomonas aeruginosa PAK chromosome, complete genome | 2455 | 2455 | 98% | 0 | 81% | CP020659.1 |
| Pseudomonas aeruginosa strain GIMC5002: PAT-169 chromosome | 2455 | 2455 | 98% | 0 | 81% | CP043549.1 |
| Pseudomonas aeruginosa strain M1608, complete genome | 2455 | 2455 | 98% | 0 | 81% | CP008862.2 |
| Pseudomonas aeruginosa strain M37351, complete genome | 2455 | 2455 | 98% | 0 | 81% | CP008863.1 |
| Pseudomonas aeruginosa strain PA-VAP-3 chromosome | 2455 | 2455 | 98% | 0 | 81% | CP028330.1 |
| Pseudomonas aeruginosa VRFPA04, complete genome | 2449 | 2449 | 98% | 0 | 81% | CP008739.2 |
| Pseudomonas aeruginosa strain AR_0095 chromosome, complete genome | 2438 | 2438 | 98% | 0 | 81% | CP027538.1 |
| Pseudomonas otitidis MrB4 DNA, complete genome | 2422 | 2422 | 98% | 0 | 81% | AP022642.1 |

TABLE 6
Non-Pseudomonas Species & Strains Comprising an Orthologue of PSPTO_820 (BLASTN Results)

| | Max | Total | Query | E | Per. | |
|---|---|---|---|---|---|---|
| Description | Score | Score | Cover | value | Ident | Accession |
| Azotobacter chroococcum strain B3, complete genome | 2942 | 2942 | 98% | 0 | 84% | CP011835.1 |
| Azotobacter chroococcum NCIMB 8003, complete genome | 2935 | 2935 | 98% | 0 | 84% | CP010415.1 |
| Azotobacter salinestris strain KACC 13899 chromosome, complete genome | 2795 | 2795 | 98% | 0 | 83% | CP045302.1 |
| Lysobacter gummosus strain 3.2.11, complete genome | 2497 | 2497 | 97% | 0 | 82% | CP011131.1 |
| Variovorax sp. PBL-H6 genome assembly, chromosome: 1 | 2473 | 2473 | 98% | 0 | 81% | LR594659.1 |
| Xanthomonas arboricola pv. juglandis strain Xaj 417 genome | 2431 | 2431 | 98% | 0 | 81% | CP012251.1 |
| Xanthomonas arboricola strain 17, complete genome | 2431 | 2431 | 98% | 0 | 81% | CP011256.1 |
| Xanthomonas arboricola pv. pruni strain 15-088 chromosome, complete genome | 2425 | 2425 | 98% | 0 | 81% | CP044334.1 |
| Burkholderia cenocepacia MC0-3 chromosome 3, complete sequence | 2438 | 2438 | 98% | 0 | 81% | CP000960.1 |
| Xanthomonas citri pv. glycines strain 2098 chromosome, complete genome | 2409 | 2409 | 98% | 0 | 81% | CP041965.1 |
| Burkholderia cenocepacia AU 1054 chromosome 1, complete sequence | 2412 | 2412 | 98% | 0 | 81% | CP000378.1 |
| Burkholderia cenocepacia HI2424 chromosome 3, complete sequence | 2412 | 2412 | 98% | 0 | 81% | CP000460.1 |
| Burkholderia cenocepacia strain CR318 chromosome 3, complete sequence | 2412 | 2412 | 98% | 0 | 81% | CP017240.1 |
| Xanthomonas axonopodis pv. phaseoli strain ISO18C8, complete genome | 2392 | 2392 | 98% | 0 | 81% | CP012063.1 |
| Xanthomonas axonopodis pv. phaseoli strain ISO98C12, complete genome | 2392 | 2392 | 98% | 0 | 81% | CP012057.1 |
| Xanthomonas sp. ISO98C4, complete genome | 2392 | 2392 | 98% | 0 | 81% | CP012060.1 |
| Burkholderia cenocepacia strain FDAARGOS_720 chromosome 1 | 2407 | 2407 | 98% | 0 | 81% | CP050980.1 |
| Paraburkholderia terricola strain mHS1 chromosome mHS1_A, complete | 2399 | 2399 | 98% | 0 | 81% | CP024941.1 |
| sequence | | | | | | |
| Xanthomonas axonopodis pv. dieffenbachiae LMG 695 genome | 2375 | 2375 | 98% | 0 | 81% | CP014347.1 |
| Ralstonia solanacearum strain UA-1591 chromosome | 2383 | 2383 | 98% | 0 | 81% | CP034195.1 |
| Paraburkholderia sprentiae WSM5005 chromosome 1, complete sequence | 2386 | 2386 | 98% | 0 | 81% | CP017561.1 |
| Variovorax paradoxus S110 chromosome 2, complete sequence | 2374 | 2374 | 97% | 0 | 81% | CP001636.1 |
| Cupriavidus basilensis strain 4G11 chromosome secondary, complete sequence | 2372 | 2888 | 98% | 0 | 81% | CP010537.1 |
| Xanthomonas campestris pv. campestris MAFF302021 DNA, complete genome | 2364 | 2364 | 98% | 0 | 81% | AP019684.1 |
| Burkholderia lata strain A05 chromosome 3, complete sequence | 2368 | 2368 | 98% | 0 | 81% | CP024945.1 |
| Burkholderia pyrrocinia strain mHSR5 chromosome mHSR5_B, complete | 2351 | 2351 | 97% | 0 | 81% | CP024903.1 |
| sequence | | | | | | |
| Ralstonia pseudosolanacearum strain CRMRs218, complete genome | 2359 | 2359 | 98% | 0 | 81% | CP021764.1 |
| Xanthomonas euvesicatoria strain LMG930, complete genome | 2348 | 2348 | 98% | 0 | 81% | CP018467.1 |
| Burkholderia ambifaria MC40-6 chromosome 3, complete sequence | 2355 | 2355 | 98% | 0 | 81% | CP001027.1 |
| Xanthomonas perforans strain LH3 chromosome, complete genome | 2331 | 2331 | 98% | 0 | 81% | CP018475.1 |
| Cupriavidus taiwanensis isolate Cupriavidus taiwanensis STM 3679 genome | 2322 | 2322 | 97% | 0 | 81% | LT984803.1 |
| assembly, chromosome: I | | | | | | |
| Cupriavidus necator N-1 plasmid pBB1, complete sequence | 2316 | 2316 | 98% | 0 | 80% | CP002879.1 |

TABLE 7
Inventory of Predicted MDR Transporters from the RND
(Pfam PF00873) Superfamily Encoded in PsPto genome

| NCBI ID | NCBI gene description | Notes |
|---|---|---|
| RND-type: | | |
| PSPTO_0375 | Cation efflux family protein | |
| * | AcrB/AcrD/AcrF family protein | |
| PSPTO_0820 | | |
| PSPTO_1308 | AcrB/AcrD/AcrF family protein | |
| PSPTO_2592 | Aliphatic isothiocyanate | Putative ortholog of |
| | resistance protein SaxG; | P. aeruginosa PAOI |
| | AcrB/AcrD/AcrF family | MexD |
| PSPTO_2755 | AcrB/AcrD/AcrF family protein | |
| PSPTO_3100 | Aliphatic isothiocyanate | Putative ortholog of |
| | resistance protein SaxF; | P. aeruginosa PAOI |
| | AcrB/AcrD/AcrF family | MexF |
| PSPTO_3302 | AcrB/AcrD/AcrF family protein | |
| PSPTO_4304 | Isothiocyanate resistance | Putative ortholog of |
| | protein SaxB | P. aeruginosa PAOI |
| | | MexB |
| PSPTO_5191 | TpsC transporter | |

TABLE 8
Summary of log reduction in the pathogen Pto DC3000 load by the active GB ™ compared
with the control GB ™ (see Example 1)

| | | Reduction | | | Log reduction |
|---|---|---|---|---|---|
| | | in target | Percentage (%) | | in pathogen |
| | Average | pathogen load | Reduction in | Log | (Pto DC3000) |
| Experiment | CFU/cm² | (CFU/cm²) | Pto DC3000 load | (CFU/cm²) | load* |
| Biological replicate-1 | | | | | |
| Pto DC3000 control | 2.0E+05 | — | | 5 | — |
| Plant control GB ™ 1 | 1.19E+05 | — | | 5 | — |
| Plant active GB ™ 1 | 5.5E+03 | 1.14E+05 | 95 | 4 | 1.3 |
| Biological replicate-2 | | | | | |
| Pto DC3000 control | 1.6E+04 | — | | 4 | — |
| Plant control GB ™ 1 | 1.6E+04 | — | | 4 | — |
| Plant active GB ™ 1 | 2.7E+02 | 1.6E+04 | 98 | 2 | 1.8 |
| Biological replicate-3 | | | | | |
| Pto DC3000 control | 6.9E+04 | — | | 5 | — |
| Plant control GB ™ 1 | 3.0E+05 | — | | 5 | — |
| Plant active GB ™ 1 | 2.9E+03 | 3.0E+05 | 99 | 3 | 2.0 |
| Average log reduction | | | | | 1.7 |
| Biological replicate-1 | | | | | |
| Pto DC3000 control | 6.8E+04 | — | | 5 | — |
| Plant control GB ™ 2 | 3.2E+05 | — | | 6 | — |
| Plant active GB ™ 2 | 1.4E+04 | 3.0E+05 | 96 | 4 | 1.4 |
| Biological replicate 2 | | | | | |
| Pto DC3000 control | 4.6E+05 | — | | 6 | — |
| Plant control GB ™ 2 | 9.0E+04 | — | | 5 | — |
| Plant active GB ™ 2 | 7.2E+03 | 8.3E+04 | 92 | 4 | 1.1 |
| Biological replicate 3 | | | | | |
| Pto DC3000 control | 4.8E+05 | — | | 6 | — |
| Plant control GB ™ 2 | 7.2E+04 | — | | 5 | — |
| Plant active GB ™ 2 | 6.3E+03 | 6.5E+04 | 91 | 4 | 1.1 |
| Average log reduction | | | | | 1.2 |

TABLE 9
Durability of Anti-Bacterial Response Delivered by Conjugation

| | | | Percentage | | |
|---|---|---|---|---|---|
| | | | (%) kill | % kill | |
| | CFU/mL | CFU/mL | active GB- | active | Difference |
| Experiment | Day 1 | Day 7 | Day 1 | GB-Day 7 | in % kill |
| Pfu 896 Guided Biotic | | | | | |
| vs. Pto DC3000 | | | | | |
| Experiment 1 | | | | | |
| Pathogen only. Pto DC3000 | 8.05E+03 | 4.69E+05 | | | |
| Non Active Guided Biotic | 2.06E+04 | 1.16E+05 | | | |
| Active Guided Biotic | 2.49E+02 | 1.94E+03 | 98.8 | 98.3 | −0.5 |
| Experiment 2 | | | | | |
| Pathogen only. Pto DC3000 | 7.68E+02 | 1.57E+04 | | | |
| Non Active Guided Biotic | 8.16E+02 | 6.34E+04 | | | |
| Active Guided Biotic# | 3.26E+01 | 1.00E+00 | 96.0 | 100.0 | 4 |
| Experiment 3 | | | | | |
| Pathogen only. Pto DC3000 | 7.40E+02 | 3.15E+05 | | | |
| Non-Active Guided Biotic | 9.16E+04 | 1.16E+06 | | | |
| Active Guided Biotic | 1.36E+02 | 1.08E+04 | 99.9 | 99.1 | −0.8 |
| Average % kill | | | | | 0.9 |
| Pfu 887 Guided Biotic | | | | | |
| vs. Pto. DC3000 | | | | | |
| Experiment 1 | | | | | |
| Pathogen only. Pto DC3000 | 7.40E+02 | 3.15E+05 | | | |
| Non-Active Guided Biotic | 2.57E+04 | 1.17E+06 | | | |
| Active Guided Biotic | 1.80E+02 | 1.27E+04 | 99.3 | 98.9 | −0.4 |
| Experiment 2 | | | | | |
| Pathogen only. Pto DC3000 | 1.15E+04 | 1.96E+06 | | | |
| Non-Active Guided Biotic | 9.17E+03 | 1.55E+05 | | | |
| Active Guided Biotic | 6.38E+02 | 1.97E+03 | 93.0 | 98.7 | 5.7 |
| Experiment 3 | | | | | |
| Pathogen only. Pto DC3000 | 1.15E+04 | 1.96E+06 | | | |
| Non-Active Guided Biotic | 1.48E+03 | 2.45E+05 | | | |
| Active Guided Biotic | 3.15E+02 | 4.71E+03 | 78.8 | 98.1 | 19.3 |
| Average % kill | | | | | 8.2 |

Formula/key:
Percentage (%) kill by active GB = (CFU/mL of nonactive GB − CFU/mL of active GB)/CFU/mL of nonactive GB*100
Difference in % killing = Percentage (%) kill active GB at Day 7 − % kill active GB at Day 1)
Average % kill = Average of difference in % kill of triplicate experiments
#0E+0 changed to 1E+0 to calculate log CFU/mL

NUCLEOTIDE AND PROTEIN SEQUENCES

>PSPTO_0820 (SEQ ID NO: 1)
ATGAGCGAAGGTCGTTTCAACCTGTCAGTGCTGGCCGTGCGCGAGCGCTCGATCACCCTG
TTCCTGATTTGCCTGATTTCGCTGGCCGGGGTCATTGCCT
TTTTCAAACTGGGCCGCGCCGAAGACCCGGCCTTCACGGTCAAGGTAATGACCGTGGTGT
CGGTCTGGCCGGGCGCAACCGCCCAGGAGATGCAGGATCA
GGTGGCGGAGAAGATCGAAAAGCGCCTTCAGGAACTGCGCTGGTACGACCGCACCGAAA
CCTACACGCGGCCTGGCATGGCATTCACAACCCTGACCCTG
CTCGACAGCACGCCGCCGTCGCAAGTGCCGGATGAGTTTTATCAGGCACGCAAGAAAAT
CGGTGACGAGGCCATGACGCTTCCGGCCGGGGTGATCGGGC
CGATGGTCAACGACGAGTATTCGGACGTTACTTTCGCGCTGTTCGCGCTCAAGGCCAAAG
GCGAGCCGCAGCGCGTGCTGGCACGTGACGCCGAATCGCT
GCGCCAGCGCCTGCTGCATGTGCCGGGCGTGAAGAAGGTCAACATCGTGGGCGAGCAGC
CCGAGCGCATCTACGTCGAGTTCTCCCACGAGCGACTGGCA
ACGCTGGGTATCAGCCCGCAAGAGGTATTTGCCGCGCTGAATAATCAGAATGCGCTTACC
CCGGCAGGCTCGGTCGAAACCCGTGGGCCGCAGGTGTTCA
TTCGGCTCGACGGCGCTTTCGATGAGCTGCAGAAGATCCGCGATACGCCGGTTGTGGCTC
AGGGCCGCACGCTGAAGCTGGCGGACATTGCCACGGTCAA
ACGCGGTTACGAAGACCCGGCAACGTTCATGATTCGCAACGGCGGCGAGCCGGCACTGT
TGCTGGGGATCGTCATGCGCGATGGCTGGAACGGGCTGGAC
CTTGGAAAGGCGCTGGATCATGAGGTGGGCGCGATCAACGCCGAGCTGCCCTTGGGCAT
GAGCCTGAACAAGGTCACGGACCAGGCCGTCAACATCAGTT
CGGCGGTCGATGAGTTCATGATCAAGTTTTTCGTCGCATTGCTGGTGGTCATGCTGGTCT
GCTTTATCAGCATGGGCTGGCGTGTGGGCGTTGTGGTGGC
TGCCGCCGTACCGCTGACCCTGGCGGTGGTCTTCGTGATCATGGCCATGAGCGGCAAGAA
TTTCGACCGCATTACATTGGGTTCACTGATTCTGGCGCTC
GGGCTGCTGGTCGACGACGCGATCATCGCCATCGAAATGATGGTGGTGAAGATGGAAGA
AGGTTACGACCGCATCGCGGCCTCTGCGTACGCCTGGAGCC
ACACCGCCGCGCCCATGTTATCCGGCACCCTGGTCACCGCTGTCGGCTTCATGCCCAACG
GTTTTGCGCGCTCCACGGCAGGCGAATACACCAGCAACAT
GTTCTGGATCGTCGGTATCGCGCTGATTGCCTCATGGGTGGTCGCGGTGTTTTTCACACCG
TATCTGGGCGTGAAACTGTTGCCTGAGGTGAAGCAGGTC
GAAGGCGGACATGCAACGCTTTACGACACCCCACGCTACAACCGTTTCCGCCGGGTTCTG
GCACGCGTCATTGCAGGCAAGTGGCTGGTCGCAGGTTCGG
TCATCGGGTTGTTCGTCCTGGCAGTGCTGGGCATGGGGCTGGTCAAGAAACAGTTTTTTC
CGGTGTCCGACCGCCCAGAGGTGCTGGTCGAACTGCAGAT
GCCTTACGGCACCTCGATTGCTCAAACCAGCGCGGCCGCGGCCAAAGTGGAAAGCTGGC
TGGCCGAGCAGGCAGAAGCCGGGATCGTCACCGCCTACATT
GGCCAGGGCGCGCCACGTTTCTACATGGCGATGGGGCCGGAATTACCTGACCCGTCATTT
GCCAAGATCGTGGTGCGCACCGACAGCCAGGAACAGCGCG
AGACACTGAAACACCGCTTGCGTCAGGCTATTTCCGAAGGGCTGGCTGGCGAGGCGCAA
GTGCGCGTCACGCAACTGGTCTTCGGCCCGTATTCACCCTA
CCCGGTCGCCTACCGCGTTACTGGCCATGACCCGGACACACTGCGCAGCATTGCGGCGCA
GGTGCAACAGGTGCTGAGCGCCAGCCCGATGATGCGCACC
GTCAATACTGACTGGGGCACGCGCACCCCAACGCTGCATTTCACCTTGCAACAGGACCG
GATGCAGGCCATCGGGTTGAGTTCCAGCCAGGTCGCGCAAC
AATTGCAGTTCCTGCTGACCGGCCTGCCGGTTACGGCGGTGCGCGAGGACATTCGCACCG
TGCAGGTGGTTGCCCGCTCGGCTGGCGACACCCGACTGGA
TCCGGCAAAAATCATGGACTTCACCCTCACAGGCGTCGATGGGCAACGTGTTCCGCTGTC
GCAGATCGGTGCAGTCGATGTGCGCATGGAAGAGCCGGTC
ATGCGCCGGCGCGACCGCACGCCAACCATCACCGTACGGGGCGACATCGCCGACGGCCT
GCAACCGCCAGATGTATCGACGGCCATTACCCGGCAGTTGC
AGCCCATCATCGACACGCTGCCCAGTGGCTATCGGATCGATCAGGCAGGTTCAATCGAG
GAATCCGGCAAGGCAATGGCGGCGATGTTGCCACTGTTCCC
GATCATGCTGGCGGTCACGCTGATCATCCTGATTCTGCAGGTGCGTTCGATATCGGCCAT
GGTCATGGTGTTTCTGACCAGCCCGCTGGGGCTGATCGGT
GTGGTGCCTACGCTGATCCTCTTTCAGCAGCCCTTCGGCATCAATGCACTGGTCGGGCTG
ATCGCACTGTCCGGCATTCTGATGCGCAACACGCTGATCC
TGATCGGCCAGATCCACCACAATGAACAGGCGGGGCTCGACCCGTTTCAGGCCGTGGTC
GAAGCCACCGTACAACGTGCGCGCCCGGTGATACTGACAGC
GCTGGCCGCCATTCTGGCGTTTATCCCCCTCACCCATTCGGTGTTCTGGGGCACGCTGGCC
TACACGCTGATCGGCGGCACATTCGCCGGTACGGTGCTG
ACCCTGGTGTTTCTGCCGGCAATGTACTCGATCTGGTTCAGGATCAGGCCCGATGGCAAC
GAGCGGCCGCAAGGCGGTCATTCCTTGTCCACAGGTAAAG GGGTGAGCTAG
>AcrB/AcrD/AcrF family protein (encoded by PSPTO_0820)(SEQ ID NO: 2)
MSEGRFNLSVLAVRERSITLFLICLISLAGVIAFFKLGRAEDPAFTVKVMTVVSVWPGATAQE
MQDQVAEKIEKRLQELRWYDRTETYTRPGMAFTTLTLLDSTPPSQVPDEFYQARKKIGDEA
MTLPAGVIGPMVNDEYSDVTFALFALKAKGEPQRVLARDAESLRQRLLHVPGVKKVNIVGE
QPERIYVEFSHERLATLGISPQEVFAALNNQNALTPAGSVETRGPQVFIRLDGAFDELQKIRDT
PVVAQGRTLKLADIATVKRGYEDPATFMIRNGGEPALLLGIVMRDGWNGLDLGKALDHEVG
AINAELPLGMSLNKVTDQAVNISSAVDEFMIKFFVALLVVMLVCFISMGWRVGVVVAAAVP
LTLAVVFVIMAMSGKNFDRITLGSLILALGLLVDDAIIAIEMMVVKMEEGYDRIAASAYAWS
HTAAPMLSGTLVTAVGFMPNGFARSTAGEYTSNMFWIVGIALIASWVVAVFFTPYLGVKLLP
EVKQVEGGHATLYDTPRYNRFRRVLARVIAGKWLVAGSVIGLFVLAVLGMGLVKKQFFPVS
DRPEVLVELQMPYGTSIAQTSAAAAKVESWLAEQAEAGIVTAYIGQGAPRFYMAMGPELPD
PSFAKIVVRTDSQEQRETLKHRLRQAISEGLAGEAQVRVTQLVFGPYSPYPVAYRVTGHDPD
TLRSIAAQVQQVLSASPMMRTVNTDWGTRTPTLHFTLQQDRMQAIGLSSSQVAQQLQFLLT
GLPVTAVREDIRTVQVVARSAGDTRLDPAKIMDFTLTGVDGQRVPLSQIGAVDVRMEEPVM
RRRDRTPTITVRGDIADGLQPPDVSTAITRQLQPIIDTLPSGYRIDQAGSIEESGKAMAAMLPLF
PIMLAVTLIILILQVRSISAMVMVFLTSPLGLIGVVPTLILFQQPFGINALVGLIALSGILMRNTLI
LIGQIHHNEQAGLDPFQAVVEATVQRARPVILTALAAILAFIPLTHSVFWGTLAYTLIGGTFAG
TVLTLVFLPAMYSIWFRIRPDGNERPQGGHSLSTGKGVS
>PSPTO_4977 (SEQ ID NO: 3)
ATGTTGCGCAAACTTTCGTTGGTCGTGGCTGTTTCGTTGGCGTCCAGCGGACTGACCTGG
GCTGCCGACTTGCCGCTGCCAACCAAAACCGGTCTGTTGA
ATGTGTATCAGCAGGCGGTAGACAACAACGCCGACCTCGCGGCCTCGCGTGCCGATTAC
GATGCCCGCAAGGAAGCCGTGCCACAGGCCCGAGCCGGCCT
GCTGCCGAATATTTCCGGCAGTGTCCAGAACACCAACACCCGCACCAGCATCGACCGCC
CCAGCGCCGTGGCGACCCGCAGCGGCACGGTTTATCAGGCC
ACCCTGAGCCAGCCGATCTTTCGCGCCGACCGCTGGTTCCAGTTGCAGGCTGCCGAAGCG
GTCAACGAACAGGCCGCGCTGGAACTGTCGGCCACCGAGC
AGAACCTGATCCTGCAATCGGCGCAGAGCTATTTCAGTGTGTTGCGCGCGCAGGACAATC
TGGCCTCGACCAAGGCTGAGGAAGCGGCGTTCAAACGCCA
GCTCGATCAGGCCAACGAACGCTTCGATGTCGGTCTGTCAGACAAGACCGATGTGCTGC
AGGCCCAGGCCAGCTACGACACCTCGCGCGCCAGCCGGCTG
ATCGCCAGGCGTCAGGTGGACGATGCCTTTCAGGCGCTGGTGACCCTGACCAATCGCGA
ATACAACTCCATCGAAGGCATCGTGCACACCTTGCCGGTGC
TGGCACCAACGCCCAACGACGCCAAGGCCTGGGTGGATACGGCAGCGCAACAAAACCTC
AACCTGCTGGCCAGCAACTACGCCGTCAGCGCTGCCGAAGA
AACCCTGCGCCAGCGCAAGGCCGGGCACGCGCCCACCCTTGATGCCGTGGCGACTTACC
AGCGTGGCGACAACGATGCATTGGGTTTCAACAACCCCAAC
TACACCGGGCAAAATTACGGCGGCGACGTCGAGCAACGCAGCATTGGCGTGCAGTTGAA
TATCCCGATCTACAGCGGCGGCCTGACCAGTTCACAGGTGC
GTGAGGCTTATTCGCGCCTGAGCCAGAGCGAGCAGCGCCGCGAAAGCCTGCGACGTCAG
GTGGTGGAAAACACCCGTAACCTGCACCGTGCGGTGAACAC
TGATGTCGAGCAGGTTCAGGCGCGCAAACAGTCGATCATCTCCAACCAGAGTGCGCTGG
AAGCCACGGAAATCGGCTATCAGGTCGGCACCCGCAACATC
GTCGATGTGCTGGACGCCCAGCGTCAGTTGTATGCCTCGGTGCGTGACTACAACAACACG
CGCTATGACTACATCCTCGACAACCTGCGCCTCAAGCAGG
CAGCGGGCACCCTGAACCCGGGCGACTTGCAGGACCTGTCACGCTACCTCAAACCGGAC
TACAACCCGGACAAGGACTTCCTGCCGCCGGATTTGGCGAC
TGCAGCGCAGAAGAATTTCGAGCGGCCGGCGCAGCGCTGA
>Outer membrane efflux protein TolC (encoded by PSPTO_4977)(SEQ ID NO: 4)
MLRKLSLVVAVSLASSGLTWAADLPLPTKTGLLNVYQQAVDNNADLAASRADYDARKEAV
PQARAGLLPNISGSVQNTNTRTSIDRPSAVATRSGTVYQATLSQPIFRADRWFQLQAAEAVNE
QAALELSATEQNLILQSAQSYFSVLRAQDNLASTKAEEAAFKRQLDQANERFDVGLSDKTDV
LQAQASYDTSRASRLIARRQVDDAFQALVTLTNREYNSIEGIVHTLPVLAPTPNDAKAWVDT
AAQQNLNLLASNYAVSAAEETLRQRKAGHAPTLDAVATYQRGDNDALGFNNPNYTGQNYG
GDVEQRSIGVQLNIPIYSGGLTSSQVREAYSRLSQSEQRRESLRRQVVENTRNLHRAVNTDVE
QVQARKQSIISNQSALEATEIGYQVGTRNIVDVLDAQRQLYASVRDYNNTRYDYILDNLRLK
QAAGTLNPGDLQDLSRYLKPDYNPDKDFLPPDLATAAQKNFERPAQR

1. A method of killing a bacterial target cell, the cell comprising at least one Resistance-Nodulation-Cell Division (RND)-efflux pump, the method comprising contacting the target cell with a carrier bacterial cell, wherein the carrier cell comprises a conjugative plasmid, the plasmid encoding an antibacterial agent that is toxic to the target cell, wherein the carrier cell conjugates to the target cell and the plasmid is transferred into the target cell, wherein the agent is expressed in the target cell and the target cell is killed.

2-38. (canceled)


SEQUENCE LISTING (4 sequences)

SEQ ID NO: 1 - DNA, 3111 bp, Pseudomonas syringae
atgagcgaaggtcgtttcaacctgtcagtgctggccgtgcgcgagcgctcgatcaccctgttcctgatttgcctgatttcgctggccggggtcattgcctttttcaaactgggccgcgccgaagacccggccttcacggtcaaggtaatgaccgtggtgtcggtctggccgggcgcaaccgcccaggagatgcaggatcaggtggcggagaagatcgaaaagcgccttcaggaactgcgctggtacgaccgcaccgaaacctacacgcggcctggcatggcattcacaaccctgaccctgctcgacagcacgccgccgtcgcaagtgccggatgagttttatcaggcacgcaagaaaatcggtgacgaggccatgacgcttccggccggggtgatcgggccgatggtcaacgacgagtattcggacgttactttcgcgctgttcgcgctcaaggccaaaggcgagccgcagcgcgtgctggcacgtgacgccgaatcgctgcgccagcgcctgctgcatgtgccgggcgtgaagaaggtcaacatcgtgggcgagcagcccgagcgcatctacgtcgagttctcccacgagcgactggcaacgctgggtatcagcccgcaagaggtatttgccgcgctgaataatcagaatgcgcttaccccggcaggctcggtcgaaacccgtgggccgcaggtgttcattcggctcgacggcgctttcgatgagctgcagaagatccgcgatacgccggttgtggctcagggccgcacgctgaagctggcggacattgccacggtcaaacgcggttacgaagacccggcaacgttcatgattcgcaacggcggcgagccggcactgttgctggggatcgtcatgcgcgatggctggaacgggctggaccttggaaaggcgctggatcatgaggtgggcgcgatcaacgccgagctgcccttgggcatgagcctgaacaaggtcacggaccaggccgtcaacatcagttcggcggtcgatgagttcatgatcaagtttttcgtcgcattgctggtggtcatgctggtctgctttatcagcatgggctggcgtgtgggcgttgtggtggctgccgccgtaccgctgaccctggcggtggtcttcgtgatcatggccatgagcggcaagaatttcgaccgcattacattgggttcactgattctggcgctcgggctgctggtcgacgacgcgatcatcgccatcgaaatgatggtggtgaagatggaagaaggttacgaccgcatcgcggcctctgcgtacgcctggagccacaccgccgcgcccatgttatccggcaccctggtcaccgctgtcggcttcatgcccaacggttttgcgcgctccacggcaggcgaatacaccagcaacatgttctggatcgtcggtatcgcgctgattgcctcatgggtggtcgcggtgtttttcacaccgtatctgggcgtgaaactgttgcctgaggtgaagcaggtcgaaggcggacatgcaacgctttacgacaccccacgctacaaccgtttccgccgggttctggcacgcgtcattgcaggcaagtggctggtcgcaggttcggtcatcgggttgttcgtcctggcagtgctgggcatggggctggtcaagaaacagttttttccggtgtccgaccgcccagaggtgctggtcgaactgcagatgccttacggcacctcgattgctcaaaccagcgcggccgcggccaaagtggaaagctggctggccgagcaggcagaagccgggatcgtcaccgcctacattggccagggcgcgccacgtttctacatggcgatggggccggaattacctgacccgtcatttgccaagatcgtggtgcgcaccgacagccaggaacagcgcgagacactgaaacaccgcttgcgtcaggctatttccgaagggctggctggcgaggcgcaagtgcgcgtcacgcaactggtcttcggcccgtattcaccctacccggtcgcctaccgcgttactggccatgacccggacacactgcgcagcattgcggcgcaggtgcaacaggtgctgagcgccagcccgatgatgcgcaccgtcaatactgactggggcacgcgcaccccaacgctgcatttcaccttgcaacaggaccggatgcaggccatcgggttgagttccagccaggtcgcgcaacaattgcagttcctgctgaccggcctgccggttacggcggtgcgcgaggacattcgcaccgtgcaggtggttgcccgctcggctggcgacacccgactggatccggcaaaaatcatggacttcaccctcacaggcgtcgatgggcaacgtgttccgctgtcgcagatcggtgcagtcgatgtgcgcatggaagagccggtcatgcgccggcgcgaccgcacgccaaccatcaccgtacggggcgacatcgccgacggcctgcaaccgccagatgtatcgacggccattacccggcagttgcagcccatcatcgacacgctgcccagtggctatcggatcgatcaggcaggttcaatcgaggaatccggcaaggcaatggcggcgatgttgccactgttcccgatcatgctggcggtcacgctgatcatcctgattctgcaggtgcgttcgatatcggccatggtcatggtgtttctgaccagcccgctggggctgatcggtgtggtgcctacgctgatcctctttcagcagcccttcggcatcaatgcactggtcgggctgatcgcactgtccggcattctgatgcgcaacacgctgatcctgatcggccagatccaccacaatgaacaggcggggctcgacccgtttcaggccgtggtcgaagccaccgtacaacgtgcgcgcccggtgatactgacagcgctggccgccattctggcgtttatccccctcacccattcggtgttctggggcacgctggcctacacgctgatcggcggcacattcgccggtacggtgctgaccctggtgtttctgccggcaatgtactcgatctggttcaggatcaggcccgatggcaacgagcggccgcaaggcggtcattccttgtccacaggtaaaggggtgagctag

SEQ ID NO: 2 - protein, 1036 aa, Pseudomonas syringae
MSEGRFNLSVLAVRERSITLFLICLISLAGVIAFFKLGRAEDPAFTVKVMTVVSVWPGATAQEMQDQVAEKIEKRLQELRWYDRTETYTRPGMAFTTLTLLDSTPPSQVPDEFYQARKKIGDEAMTLPAGVIGPMVNDEYSDVTFALFALKAKGEPQRVLARDAESLRQRLLHVPGVKKVNIVGEQPERIYVEFSHERLATLGISPQEVFAALNNQNALTPAGSVETRGPQVFIRLDGAFDELQKIRDTPVVAQGRTLKLADIATVKRGYEDPATFMIRNGGEPALLLGIVMRDGWNGLDLGKALDHEVGAINAELPLGMSLNKVTDQAVNISSAVDEFMIKFFVALLVVMLVCFISMGWRVGVVVAAAVPLTLAVVFVIMAMSGKNFDRITLGSLILALGLLVDDAIIAIEMMVVKMEEGYDRIAASAYAWSHTAAPMLSGTLVTAVGFMPNGFARSTAGEYTSNMFWIVGIALIASWVVAVFFTPYLGVKLLPEVKQVEGGHATLYDTPRYNRFRRVLARVIAGKWLVAGSVIGLFVLAVLGMGLVKKQFFPVSDRPEVLVELQMPYGTSIAQTSAAAAKVESWLAEQAEAGIVTAYIGQGAPRFYMAMGPELPDPSFAKIVVRTDSQEQRETLKHRLRQAISEGLAGEAQVRVTQLVFGPYSPYPVAYRVTGHDPDTLRSIAAQVQQVLSASPMMRTVNTDWGTRTPTLHFTLQQDRMQAIGLSSSQVAQQLQFLLTGLPVTAVREDIRTVQVVARSAGDTRLDPAKIMDFTLTGVDGQRVPLSQIGAVDVRMEEPVMRRRDRTPTITVRGDIADGLQPPDVSTAITRQLQPIIDTLPSGYRIDQAGSIEESGKAMAAMLPLFPIMLAVTLIILILQVRSISAMVMVFLTSPLGLIGVVPTLILFQQPFGINALVGLIALSGILMRNTLILIGQIHHNEQAGLDPFQAVVEATVQRARPVILTALAAILAFIPLTHSVFWGTLAYTLIGGTFAGTVLTLVFLPAMYSIWFRIRPDGNERPQGGHSLSTGKGVS

SEQ ID NO: 3 - DNA, 1440 bp, Pseudomonas syringae
atgttgcgcaaactttcgttggtcgtggctgtttcgttggcgtccagcggactgacctgggctgccgacttgccgctgccaaccaaaaccggtctgttgaatgtgtatcagcaggcggtagacaacaacgccgacctcgcggcctcgcgtgccgattacgatgcccgcaaggaagccgtgccacaggcccgagccggcctgctgccgaatatttccggcagtgtccagaacaccaacacccgcaccagcatcgaccgccccagcgccgtggcgacccgcagcggcacggtttatcaggccaccctgagccagccgatctttcgcgccgaccgctggttccagttgcaggctgccgaagcggtcaacgaacaggccgcgctggaactgtcggccaccgagcagaacctgatcctgcaatcggcgcagagctatttcagtgtgttgcgcgcgcaggacaatctggcctcgaccaaggctgaggaagcggcgttcaaacgccagctcgatcaggccaacgaacgcttcgatgtcggtctgtcagacaagaccgatgtgctgcaggcccaggccagctacgacacctcgcgcgccagccggctgatcgccaggcgtcaggtggacgatgcctttcaggcgctggtgaccctgaccaatcgcgaatacaactccatcgaaggcatcgtgcacaccttgccggtgctggcaccaacgcccaacgacgccaaggcctgggtggatacggcagcgcaacaaaacctcaacctgctggccagcaactacgccgtcagcgctgccgaagaaaccctgcgccagcgcaaggccgggcacgcgcccacccttgatgccgtggcgacttaccagcgtggcgacaacgatgcattgggtttcaacaaccccaactacaccgggcaaaattacggcggcgacgtcgagcaacgcagcattggcgtgcagttgaatatcccgatctacagcggcggcctgaccagttcacaggtgcgtgaggcttattcgcgcctgagccagagcgagcagcgccgcgaaagcctgcgacgtcaggtggtggaaaacacccgtaacctgcaccgtgcggtgaacactgatgtcgagcaggttcaggcgcgcaaacagtcgatcatctccaaccagagtgcgctggaagccacggaaatcggctatcaggtcggcacccgcaacatcgtcgatgtgctggacgcccagcgtcagttgtatgcctcggtgcgtgactacaacaacacgcgctatgactacatcctcgacaacctgcgcctcaagcaggcagcgggcaccctgaacccgggcgacttgcaggacctgtcacgctacctcaaaccggactacaacccggacaaggacttcctgccgccggatttggcgactgcagcgcagaagaatttcgagcggccggcgcagcgctga

SEQ ID NO: 4 - protein, 479 aa, Pseudomonas syringae
MLRKLSLVVAVSLASSGLTWAADLPLPTKTGLLNVYQQAVDNNADLAASRADYDARKEAVPQARAGLLPNISGSVQNTNTRTSIDRPSAVATRSGTVYQATLSQPIFRADRWFQLQAAEAVNEQAALELSATEQNLILQSAQSYFSVLRAQDNLASTKAEEAAFKRQLDQANERFDVGLSDKTDVLQAQASYDTSRASRLIARRQVDDAFQALVTLTNREYNSIEGIVHTLPVLAPTPNDAKAWVDTAAQQNLNLLASNYAVSAAEETLRQRKAGHAPTLDAVATYQRGDNDALGFNNPNYTGQNYGGDVEQRSIGVQLNIPIYSGGLTSSQVREAYSRLSQSEQRRESLRRQVVENTRNLHRAVNTDVEQVQARKQSIISNQSALEATEIGYQVGTRNIVDVLDAQRQLYASVRDYNNTRYDYILDNLRLKQAAGTLNPGDLQDLSRYLKPDYNPDKDFLPPDLATAAQKNFERPAQR